(19) United States
(12) Patent Application Publication
(10) Pub. No.: US 2026/0224620 A1
(43) Pub. Date: Aug. 6, 2026

(54) Combination Adoptive Immune Cell and Anti-CD47 Therapy and Related Compositions

(71) Applicant: The Board of Trustees of the Leland Stanford Junior University, Stanford, CA (US)

(72) Inventors: Crystal Mackall, Stanford, CA (US); Jennifer R. Cochran, Stanford, CA (US); Sean Yamada-Hunter, Stanford, CA (US); Johanna Theruvath, Redwood City, CA (US); Brianna McIntosh, Redwood City, CA (US)

(21) Appl. No.: 19/151,111

(22) PCT Filed: Jan. 26, 2024
(86) PCT No.: PCT/US2024/013209

§ 371 Date: Jul. 25, 2025

Related U.S. Application Data
(60) Provisional application No. 63/441,637, filed on Jan. 27, 2023.

(51) Int. Cl. A61K 35/17; A61K 35/15; A61K 38/00; A61K 40/11; A61K 40/15; A61K 40/17; A61K 40/31; A61K 40/42; A61P 35/00; C07K 16/28; C12N 15/11

(52) U.S. Cl. CPC A61K 35/17; A61K 35/15; A61K 38/00; A61K 40/11; A61K 40/15; A61K 40/17; A61K 40/31; A61K 40/421; A61P 35/00; C07K 16/2803; C07K 16/2896; C12N 15/11; A61K 2239/48; C07K 2317/72; C12N 2830/34

(57) ABSTRACT

Provided are nucleic acids encoding CD47 polypeptides. In some embodiments, the nucleic acids encode a CD47 polypeptide comprising a mutant CD47 Ig-like domain that reduces binding of a therapeutic anti-CD47 binding agent to the CD47 polypeptide as compared to binding of the therapeutic anti-CD47 binding agent to a wild-type CD47 polypeptide, and where the CD47 polypeptide retains binding to SIRPα. In certain embodiments, the mutant CD47 Ig-like domain comprises a mutant BC loop. Therapeutic immune cells (e.g., CAR-T cells, etc.) comprising the nucleic acids and expressing the CD47 polypeptides on their surface are also provided, as are therapeutic methods comprising administering such cells to subjects receiving an anti-CD47 therapy, e.g., to treat cancer.

### CROSS-REFERENCE TO RELATED APPLICATIONS

This application claims the benefit of U.S. Provisional Patent Application No. 63/441,637, filed Jan. 27, 2023, which application is incorporated herein by reference in its entirety.

### STATEMENT OF GOVERNMENT SUPPORT

This invention was made with Government support under contract CA263500 awarded by the National Institutes of Health. The Government has certain rights in the invention.

### INCORPORATION BY REFERENCE OF SEQUENCE LISTING PROVIDED AS A SEQUENCE LISTING XML FILE

A Sequence Listing is provided herewith as a Sequence Listing XML, “STAN-1984WO_SEOLIST”, created on Jan. 26, 2024 and having a size of 45,714 bytes. The contents of the Sequence Listing XML are incorporated herein by reference in their entirety.

### INTRODUCTION

Myeloid cells are the most plentiful immune cells within the tumor microenvironment (TME) and there has been great interest in therapeutically targeting them for antitumor effects' 3. Increased levels of tumor associated macrophages (TAMs) are linked with poorer clinical outcomes in numerous studies^{4,5}, and substantial preclinical data demonstrates that reducing or eliminating TAMs enhances responses to chemotherapy and immunotherapy⁶⁻⁸. However, dozens of clinical studies testing CSF1R and CCR2 inhibitors, designed to deplete TAMs and tumor associated myeloid cells, have been completed or are ongoing, and thus far none have demonstrated significant clinical benefit^{2,3,9}. Alternatively, increasing TAM density is correlated with improved clinical outcomes in some cancers^{5,10}, and augmenting the phagocytic activity of TAMs by blocking the CD47/SIRPα axis mediates antitumor effects in several preclinical models¹¹⁻¹⁴. Clinical trials of agents designed to block the CD47/SIRPα axis demonstrated antitumor activity in some liquid tumors when combined with additional agents¹⁵⁻¹⁶, but clear evidence for single agent activity or activity in solid cancers is lacking^{17,18}. Thus, despite extensive effort, effective therapeutic approaches to target TAMs for clinical benefit remain elusive.

### SUMMARY

Provided are nucleic acids encoding CD47 polypeptides. In some embodiments, the nucleic acids encode a CD47 polypeptide comprising a mutant CD47 Ig-like domain that reduces binding of a therapeutic anti-CD47 binding agent to the CD47 polypeptide as compared to binding of the therapeutic anti-CD47 binding agent to a wild-type CD47 polypeptide, and where the CD47 polypeptide retains binding to SIRPα. In certain embodiments, the mutant CD47 Ig-like domain comprises a mutant BC loop. Therapeutic immune cells (e.g., CAR-T cells, etc.) comprising the nucleic acids and expressing the CD47 polypeptides on their surface are also provided, as are therapeutic methods comprising administering such cells to subjects receiving an anti-CD47 therapy, e.g., to treat cancer.

### BRIEF DESCRIPTION OF THE FIGURES

FIG. 1: CD47 blockade leads to loss of CAR T cell efficacy in vivo in a 143B osteosarcoma model. (A) 143B model treatment scheme. Mice engrafted orthotopically in the tibia periosteum with 0.5×10⁶ 143B were treated intravenously (IV) with 10×10⁶ Her2.BBζ-CAR T cells on day 5, and then intraperitoneally (IP)±B6H12 twice on days 6 and 10 (250 μg/dose). Blood was drawn on day 12 to assess CAR T cell expansion. (B) 143B tumor growth treated as described in (A). Data are the mean±SEM of n=5 mice/arm for tumor growth. (C) Survival of 143B tumor bearing animals treated as described in (A). n=5 mice/arm for survival.

FIG. 2: CD47 blockade leads to loss of CAR T cell efficacy in vivo in an MG63.3 osteosarcoma model. (A) MG63.3 model treatment scheme. Mice engrafted orthotopically in the tibia periosteum with 1×10⁶ MG63.3 were treated IP±B6H12 three times per week (400 μg/dose) starting on day 15, then IV with 10×10⁶ B7H3.BBζ- or GD2.BBζ-CAR T cells on day 21. (B) MG63.3 tumor growth treated as described in (A). Data are the mean±SEM of n=5 mice/arm for tumor growth. (C) Survival of MG63.3 tumor bearing animals treated as described in (A). n=5 mice/arm for survival.

FIG. 3: CD47 blockade leads to loss of CAR T cell efficacy in vivo in a D425 medulloblastoma model. (A) D425 treatment scheme. Mice engrafted with 0.2×10⁶ D425 cells in the cerebellum were treated±B6H12 intraperitoneally (IP) three times per week (400 μg/dose) starting on day 4. Mice were also treated intravenously (IV) with 10×10⁶ CD19.BBζ-(non-tumor targeting control) or B7H3.BBζ-(tumor targeting) CAR T cells on day 4. (B) Quantification of D425 tumor growth by BLI, treated as described in (A). Data are the mean±SEM of n=5 mice/arm for tumor growth. (C) Survival of mice in the D425 model, treated as described in (A). n=5 mice per treatment arm for survival.

FIG. 4: CD47 blockade leads to depletion of CAR T cells in the blood and tumor. (A) Representative flow cytometry plots of hCD45⁺ T cells identified in the blood and tumor in the MG63.3 model on day 30 post tumor engraftment, treated with 10×10⁶ B7H3.BBζ-CAR T cells on day 15±3 doses of B6H12 treatment (400 μg/dose). (B) Quantification of T cells (hCD45⁺) by flow cytometry from blood and tumor in the MG63.3 model, treated IV with 10×10⁶ B7H3.BBζ-CAR T cells on day 15±3 doses of B6H12 treatment (400 μg/dose; IP) collected on day 30 after tumor engraftment. Data are the mean±SD of n=3 mice.

FIG. 5: CD47 blockade, but not treatment with isotype control leads to depletion of T cells in the blood. (A) Representative flow cytometry plots of hCD45⁺ T cells identified in the blood of non-tumor bearing mice on day 5 after treatment with 5×10⁶ CD19.28ζ-CAR T cells on day 0, subsequently treated IP with either PBS, B6H12, or mIgG1 isotype control (250 μg/dose) on day 1. Data are representative of two independent experiments. (B) Quantification of hCD8+(left) and hCD4⁺ (right) T cells in the blood of mice on day 5 in the isotype control model, treated as described in (A). Data are the mean±SD of n=5 mice. (C) Quantification of T cells (hCD4⁺ and hCD8⁺) in the blood of mice on day 12 in the 143B-Her2.BBζ model, treated as described in FIG. 1A. Data are the mean±SD of n=5 mice.

FIG. 6: CD47 blockade leads to loss of TCR T cell efficacy in vivo in an A375 melanoma model after low-dose T cell treatment. (A) A375 model treatment scheme. Mice engrafted subcutaneously (SQ) with 3×10⁶ A375 were treated IV with 2×10⁶ mock or NY-ESO-1-TCR T cells on day 9±two doses of B6H12 (250 μg/dose; IP) on days 10 and 15. Blood was drawn on day 17 to assess T cell expansion. (B) A375 tumor growth treated as described in (A). Data are the mean±SEM of n=5 mice/arm for tumor growth. (C) Survival of A375 tumor bearing animals treated as described in (A). n=5 mice/arm for survival. (D) Quantification of T cells in the blood (hCD4⁺ and hCD8⁺) by flow cytometry on day 17 of mice treated as described in (A). Data are the mean±SD of n=5 mice.

FIG. 7: CD47 blockade leads to loss of TCR T cell efficacy in vivo in an A375 melanoma model after high-dose T cell treatment. (A) A375 model high-dose treatment scheme. Mice engrafted subcutaneously (SQ) with 3×10⁶ A375 were treated IV with 5×10⁶ mock or NY-ESO-1-TCR T cells on day 7±two doses of B6H12 (250 μg/dose; IP) on days 9 and 13. Blood was drawn on day 16 to assess T cell expansion. (B) A375 tumor growth of animals treated as described in (A). Data are the mean±SEM of n=5 mice/arm. (C) Quantification of T cells (hCD4⁺ and hCD8⁺) in the blood by flow cytometry on day 16 of mice treated as described in (A). Data are the mean±SD of n=5 mice.

FIG. 8: CD47 blockade leads to loss of CAR T cell efficacy in a Nalm6 leukemia model due to T cell depletion after high-dose T cell treatment. (A) High-dose CAR T-Nalm6 model treatment scheme. Mice engrafted IV with 1×10⁶ Nalm6-fLuc cells were treated±B6H12 (400 μg/dose; IP) three times per week starting on day 3. Mice were then treated IV with 1×10⁶ mock or CD19.28ζ-nLuc-CAR T cells on day 4. Mice were serially imaged by BLI for both tumor growth (fLuc signal) and T cell expansion (nLuc signal). (B) Quantification of Nalm6 tumor growth by BLI in the high-dose CAR T model, treated as described in (A). Data are the mean±SEM of n=5 mice/arm. (C) Quantification of T cell BLI in the high-dose CAR T-Nalm6 model, treated as described in (A). Data are the mean±SEM of n=5 mice/arm. (D) Quantification of T cells by flow cytometry from the spleen in the high-dose CAR T-Nalm6 model, treated as described in (A). Data are the mean±SD of n=5 mice.

FIG. 9: CD47 blockade leads to loss of CAR T cell efficacy in a Nalm6 leukemia model due to T cell depletion after low-dose T cell treatment. (A) Low-dose CAR T-Nalm6 model treatment scheme. Mice engrafted IV with 1×10⁶ Nalm6-fLuc cells were then treated IV with 0.15×10⁶ mock or CD19.28ζ-nLuc-CAR T cells on day 4. Mice were then treated±B6H12 (250 μg/dose; IP) on days 5 and 7. Mice were serially imaged by BLI for both tumor growth (fLuc signal) and T cell expansion (nLuc signal). (B) CD19.28ζ-nLuc-CAR T cell BLI in the low-dose CAR T model on day 11, treated as described in (A). (C) Quantification of Nalm6 tumor growth by BLI mice in the low-dose CAR T model, treated as described in (A). Data are the mean±SEM of n=5 mice/arm for tumor growth. (D) Survival of tumor Nalm6 bearing mice in the low-dose CAR T model, treated as described in (A). n=5 mice per treatment arm for survival.

FIG. 10: CD47 blockade leads to T cell depletion in an Fc-independent manner after high-dose T cell treatment. (A) Nalm6 with CV-1 treatment scheme. Mice engrafted IV with 1×10⁶ Nalm6-fLuc were treated IV with 1×10⁶ CD19.BBζ-nLuc-CAR T cells on day 4, ±CV-1 (dead Fc domain; 400 μg/dose; IP) three times per week starting on day 5. (B) Nalm6-CV-1 model tumor growth, treated as described in (A). Data are the mean±SEM of n=5 mice/arm for tumor growth. (C) Survival of Nalm6 tumor bearing mice, treated as described in (A). n=5 mice/arm for survival. (D) T cells in the blood (hCD4⁺ and hCD8⁺) by flow cytometry on day 11 of mice treated as described in (A). Data are the mean±SD of n=3 mice for the CD19.BBζ group and n=4 for the CD19.BBζ+CV-1 group.

FIG. 11: CD47 blockade leads to T cell depletion in an Fc-independent manner after low-dose T cell treatment. (A) Nalm6 with low-dose CAR T and CV-1 treatment scheme. Mice engrafted IV with 1×10⁶ Nalm6-fLuc were treated IV with 0.1×10⁶ CD19.28ζ-nLuc-CAR T cells on day 4, ±CV-1 (dead Fc domain; 400 μg/dose; IP) three times on days 5, 7 and 10. (B) Low-dose CAR T-Nalm6-CV-1 model tumor growth, treated as described in (A). Data are the mean±SEM of n=5 mice/arm. (C) Quantification of T cell BLI on day 9 (four days post CV-1 treatment) in the high-dose CAR T-Nalm6-CV-1 model, treated as described in FIG. 10A. (D) Quantification of T cell BLI on day 11 (six days post CV-1 treatment) in the low-dose CAR T-Nalm6-CV-1 model, treated as described in (A).

FIG. 12: CD47 knock-out (47_{KO}) on CAR T cells leads to depletion and loss of efficacy in vivo. (A) CD47 knock-out (CD47_{KO}) efficiency in primary human T cells by flow cytometry. CD47_{WT} cells are CD47_{KO} with exogenous expression of wild-type CD47. Data are representative of n>3 donors. (B) Quantification of Nalm6 tumor growth by BLI. Mice engrafted IV with 1×10⁶ Nalm6-fLuc were treated IV with 0.15×10⁶ mock, 47_{KO}- or 47_{WT}-CD19.28ζ-nLuc-CAR T cells on day 4. 47_{WT} cells are 47_{KO} with exogenous expression of WT CD47. Data are the mean±SEM of n=5 mice/arm. (C) Survival of Nalm6 bearing mice shown in (B). n=5 mice per treatment arm. (D) Quantification of T cell BLI on day 11 of tumor growth (day 7 post CAR T engraftment) of mice treated as described in (B). Data are the mean±SD of n=5 mice.

FIG. 13: CD47 blockade leads to equivalent T cell depletion as CD47 knock-out in vivo. (A) T cell depletion model scheme. Non-tumor bearing mice treated IV with 5×10⁶ or 2×10⁶ 47_{KO}-CD19.28ζ-nLuc-CAR T cells in separate experiments, with (47_{WT}) or without (47_{KO}) CD47 exogenous expression, were then treated twice±B6H12 (250 μg/dose; IP) on days 3 and 5. Mice were imaged by BLI before (day 2 or 3) and after (day 7 or 9) aCD47 treatment, and had blood drawn on day 6 or 7. (B) Example flow cytometry plots of hCD45⁺ T cells identified in the blood in the T cell depletion model, treated as described in (A). (C) Quantification of T cell BLI in the T cell depletion model, treated as described in (A), with mice treated IV with 2×10⁶ or 5×10⁶ 47_{WT}- or 47_{KO}-CD19.28ζ-nLuc-CAR T cells, as indicated. Dashed line indicates limit of detection. Data are the mean±SD of n=5 mice. (D) Quantification of CD8⁺ (left) and CD4+ (right) T cells in the blood on day 6 or 7 by flow cytometry in the T cell depletion model, treated as described in (A), with mice treated IV with 2×10⁶ or 5×10⁶ 47_{WT}- or 47_{KO}-CD19.28ζ-nLuc-CAR T cells in separate experiments. Data are the mean±SD of n=5 mice.

FIG. 14: CD47 overexpression (47_{OE}) and knock-out (47_{KO}) do not alter T cell function in vitro. (A) Expansion of CD19.28ζ-, 47_{OE}-CD19.28ζ-, AAVS1_{KO}-CD19.28ζ-, or 47_{KO}-CD19.28ζ-CAR T cells over days in culture. T cells were thawed and activated by anti-CD3/CD28 Dynabeads on day 0, CRISPR edited on day 2, transduced on days 2 and 3, and removed from activation beads on day 4. Cells were then expanded in culture in media+IL-2 over the next week. Data are mean±SD of n=3 T cell donors. (B) CAR T cell viability over days in culture. T cells were produced and grown as described in (A). Data are mean±SD of n=3 T cell donors. (C) Nalm6-GFP tumor killing by mock, CD19.28ζ-, 47_{OE}-CD19.28ζ-, AAVS1_{KO}-CD19.28ζ-, or 47_{KO}-CD19.28ζ-CAR T cells at a 1:1 E:T ratio measured via Incucyte assay. Data are mean±SD of n=3 T cell donors, with each datapoint derived from the average of n=3 triplicate wells per donor. (D) Nalm6-GFP tumor killing by mock, CD19.28ζ-, or 47_{OE}-CD19.28ζ-CAR T cells±B6H12 at a 1:1 E:T ratio measured via Incucyte assay. Data are mean±SD of n=3 T cell donors, with each datapoint derived from the average of n=3 triplicate wells per donor. (E-F) IFN-γ (E) and IL-2 (F) secretion upon co-culture for 24 h with and without Nalm6 tumor cells at a 1:1 E:T ratio of mock, CD19.28ζ-, 47_{OE}-CD19.28ζ-, AAVS1_{KO}-CD19.28ζ- or 47_{KO}-CD19.28ζ-CAR T measured via ELISA. Mock, CD19.28ζ-, and 47_{OE}-CD19.28ζ-CAR T cells were also assessed in the presence of B6H12 and Nalm6. Data are mean±SD of n=3 T cell donors, with each datapoint derived from the average of n=3 triplicate wells per donor.

FIG. 15: CD47 overexpression (47_{OE}) and knock-out (47_{KO}) do not alter expression of T cell markers after activation and exhaustion in vitro. (A-F) Annexin V (A), CD69 (B), CD39 (C), TIM3 (D), LAG3 (E), and PD1 (F) expression upon co-culture for 24 h with and without Nalm6 tumor cells at a 1:1 E:T ratio of mock, CD19.28ζ-, 47_{OE}-CD19.28ζ-, AAVS1_{KO}-CD19.28ζ- or 47_{KO}-CD19.28ζ-CAR T measured via flow cytometry. Mock, CD19.28ζ-, and 47_{OE}-CD19.28ζ-CAR T cells were also assessed in the presence of B6H12 and Nalm6. Data are mean±SD of n=3 T cell donors.

FIG. 16: CD47 over-expression on CAR T cells enhances persistence and antitumor efficacy in vivo. (A) CD47 (top left, bottom right, and bottom left) and CAR (top right) expression on T cells by flow cytometry after CD47 over-expression (47_{OE}). Data are representative of n>3 donors. (B) Quantification of Nalm6 tumor growth by BLI. Mice engrafted IV with 1×10⁶ Nalm6 were treated IV with either 3×10⁶ mock or 47_{OE}-mock, or 0.1×10⁶ CD19.28ζ- or 47_{OE}-CD19.28ζ-CAR T cells on day 4. Data are the mean±SEM of n=5 mice/arm. (C) Quantification of Nalm6 tumor growth by BLI. Mice engrafted IV with 1×10⁶ Nalm6 were treated IV with 3×10⁶ mock, 47_{OE}-mock, CD19.28ζ-, 47_{OE}-CD19.28ζ-CAR T cells on day 4. Data are the mean±SEM of n=5 mice/arm. (D) Quantification of CD4⁺ and CD8⁺ T cells on day 45 after CAR T administration in the blood of mice treated with 0.1×10⁶ (left) or 3×10⁶ (right) CD19.28ζ- or 47_{OE}-CD19.28ζ-CAR T cells, by flow cytometry. Data are the mean±SD of n=5 mice.

FIG. 17: Macrophages are required for anti-CD47 mediated T cell depletion in vivo. (A) Macrophage depletion scheme. Non-tumor bearing mice were treated with clodronate (200 μL; IV) on day 0 and αCSF1R (400 μg/dose; IP) three times per week starting on day 0 to deplete macrophages. Mice were then treated IV with 2×10⁶ CD19.28ζ-nLuc-CAR T cells on day 6, followed by a single 250 μg IP dose of B6H12 on day 7. Mice were imaged by BLI before (day 7) and after (day 9) αCD47 treatment. (B) Quantification of CD11b⁺/F4/80⁺ macrophages collected via peritoneal lavage by flow cytometry on day 13. Data are the mean±SD of n=7 (macrophage depleted) or n=9 (macrophage non-depleted) mice. (C) Quantification of T cell BLI one day after IV administration of 2×10⁶ CD19.28ζ-nLuc-CAR T cells (day 7). Data are the mean±SD of n=9 (macrophage depleted) or n=10 (macrophage non-depleted) mice. (D) Quantification of T cell BLI before (day 7) and after (day 9) treatment with B6H12 of mice in the macrophage depletion model, treated as descried in (A). Data are the mean±SD of n=4 (macrophage depleted, PBS treated) or n=5 (all other groups) mice.

FIG. 18: Human macrophages phagocytose human CAR T cells in vitro. (A) Quantification of phagocytosis of CFSE labeled CD19.28ζ-CAR T cells from three donors by primary human macrophages from three donors by flow cytometry, following one hour of co-culture. CAR T cells were either untreated or treated with B6H12 or mIgG1 isotype control prior to addition of macrophages. Data are mean±SD of n=3 T cell donors, with each datapoint derived from the average of n=3 triplicate wells per donor. (B) Quantification of phagocytosis of CFSE labeled CAR T cells by primary human macrophages by flow cytometry, following one hour of co-culture. Data are the mean±SD of n=3 triplicate wells. Data is reproducible across four different macrophage donors.

FIG. 19: CD47 expression on tumor cells and activated T cells. (A) CD47 expression on tumor cells, mock T cells, and CD19.28ζ-CAR T cells by QuantiBrite quantitative flow cytometry. T cells were assessed on day 4 and day 11 of culture. (B) Quantification of histograms shown in (A) for each cell type. Data are representative of n=1 tumor sample and n=3 T cell donors. Data are reproducible across n>3 experiments.

FIG. 20: CD47 expression on T cell subtypes. (A) Quantification of CD47 expression on CD4⁺ and CD8⁺ CD19.28ζ-CAR T cells by QuantiBrite quantitative flow cytometry. T cells were thawed on day −1, activated by anti-CD3/CD28 Dynabeads on day 0, transduced on days 2 and 3, removed from activation beads on day 4, and then expanded in culture in media+IL-2 over the next week. Cells were assessed by flow cytometry on day 0 (prior to activation), day 4 (after activation bead removal), day 7, and day 11 (time of typical transfer in vivo). Data are mean+SD of n=3 T cell donors. Only significant differences are shown. All other groups are not significantly different, comparing between the same subtype over time and between subtypes at the same timepoint. (B) Quantification of CD47 expression on T_{Naive} (T_{N}), T_{central Memory} (T_{CM}), T_{Effector Memory} (T_{EM}), and T_{Effector Memroy re-expressing CD45RA} (T_{EMRA}) CD4+ (top) and CD8⁺ (bottom) CD19.28ζ-CAR T cell subtypes on days 0, 4, 7, and 11 post-activation by QuantiBrite quantitative flow cytometry. T cells were generated as described in (A). Data are mean±SD of n=3 T cell donors. Only significant differences are shown. All other groups are not significantly different, comparing between the same subtype over time and between subtypes at the same timepoint.

FIG. 21: Calreticulin and CD47 expression on CAR T cells. (A) Calreticulin (left) and CD47 (middle) expression on CAR T cells by flow cytometry on day 10 of culture. Quantification of histograms shown on the right for each type of CAR T cell. Data are representative of n=3 donors. (B and C) Quantification of (B) calreticulin and (C) CD47 expression on CAR T cells on day 16 and day 25 of culture by flow cytometry. Data are the mean+SD of n=3 donors.

FIG. 22: Phagocytosis of T cells by macrophages in a CAR T treated patient. Microscope images of histiocytes engulfing lymphocytes collected from the CSF of a CD19.28ζ-CAR T treated large B cell lymphoma (LBCL) patient and stained with hematoxylin and eosin (1000× magnification). Lower panels are enlargements of the boxed regions of the respective upper panels.

FIG. 23: CAR mRNA⁺ myeloid cells in CAR T treated patients detected by single-cell RNA sequencing. (A) Weighted nearest neighbor (wnn) UMAP single cell landscapes color coded for cell type (left) or CAR mRNA (right). Data are derived from n=6,316 CAR⁺ cells sorted from the blood collected on day 7 after CAR T infusion of n=9 axicabtagene ciloleucel (axi-cel; CD19.28ζ) treated LBCL patients, sampled to 500 cells/patient sample. (B) UMAP single cell landscapes color coded for cell type (left) or CAR mRNA (right). Data are derived from n=25,598 cells from the CSF of n=4 GD2.BBζ treated diffuse midline glioma (DMG) patients, sampled to 500 cells/patient sample.

FIG. 24: Yeast displayed CD47 as an N-terminal fusion binds B6H12. (A) Schematic of the engineered CD47 (47_{E}) mechanism, whereby αCD47 antibodies bind tumor cells, but not 47_{E}-T cells, triggering tumor-specific phagocytosis. (B) Cartoon of yeast displayed CD47 Ig-like domain using the pCTCON2 vector. CD47 is displayed as an N-terminal fusion. (C) Binding of 100 nM B6H12 to yeast displayed CD47 in pCTCON2 by flow cytometry. Data are representative of n=3 independent experiments. (D) Binding curve of B6H12 to yeast displayed CD47 in pCTCON2, measured over multiple concentrations by flow cytometry. Data are the MFI of n=1 experiment.

FIG. 25: Yeast displayed CD47 as an N-terminal fusion does not bind SIRPα, but does bind CV-1. (A) Binding of 300 nM human (left) and mouse (right) SIRPα to yeast displayed CD47 in pCTCON2 by flow cytometry. Data are representative of n=3 independent experiments. (B) Binding of 500 nM CV-1 to yeast displayed CD47 in pCTCON2 by flow cytometry. Data are representative of n=3 independent experiments.

FIG. 26: A yeast displayed library of CD47 variants was sorted with alternating negative and positive sorts for selective binding. Flow cytometry sorting plots of all six sorts of the CD47 library, indicating negative sorts to B6H12 and positive sorts to CV-1. Collected population indicated by the black box in each plot.

FIG. 27: Engineered CD47 variants bind CV-1, but not B6H12. (A) Binding of 500 nM B6H12 or 100 nM CV-1 to the yeast displayed CD47 library population collected after sort 6 or yeast displayed WT CD47. Data are representative of n=2 independent experiments. (B) Consensus mutations identified in yeast sequenced after sorts 4, 5, and 6. Data are frequencies of identified mutations out of n=13, n=16, and n=12 sequenced clones for sorts 4, 5, and 6, respectively. (C) Binding of B6H12 and CV-1 to CD47 mutants displayed on yeast. Data are the mean±SD of n=3 individual yeast clones, normalized to MFI from binding to 47_{WT}.

FIG. 28: Yeast displayed CD47 engineered variants as C-terminal fusions bind SIRPα, but not B6H12. (A) Cartoon of yeast-displayed CD47 Ig-like domain using the pFreeNTerm (pFNT) vector. CD47 is displayed as a C-terminal fusion, along with GFP to monitor protein expression. (B) Binding of 100 nM human and mouse SIRPα to yeast displayed CD47 in pFNT by flow cytometry. Data are representative of n=3 independent experiments. (C) Binding of B6H12, CV-1, hSIRPα, and mSIRPα to yeast displayed 47_{WT}, 47_{A3OP}, and 47_{Q31P} variants. Data are the mean±SD of n=3 individual yeast clones, normalized to MFI from binding to ⁴⁷WT.

FIG. 29: Crystal structure of CD47 binding SIRPα or B6H12 reveals discrete binding mechanisms. Crystal structures of CD47 (red) binding SIRPα (dark pink, left) [PDB: 2JJS] and B6H12 (light blue, right) [PDB: 5TZU], identifying residues A30 (gold) and Q31 (blue). Lower panels are enlargements of the boxed regions in the full structures.

FIG. 30: BC loop of CD47 is amenable to mutation to retain SIRPα binding but disrupt anti-CD47 antibody binding. (A) Crystal structure of CD47 (yellow) binding SIRPα (orange) [PDB: 2JJS], identifying the CD47 BC loop (green), containing CD47 residues T26-Q31. (B) Binding of B6H12, TJC4, Hu5F9, and hSIRPα to yeast displayed 47_{WT}, 47_{A30P}, and 47_{Q31P} variants. Data are the mean±SD of n=3 individual yeast clones, normalized to MFI from binding to 47_{WT}. (C) Binding of 100 nM B6H12, 100 nM TJC4, 100 nM Hu5F9, and 100 nM hSIRPα to yeast displayed 47_{T26A}, 47_{N27A}, and 47_{M28A} variants. Data are the mean±SD of n=3 individual yeast clones, normalized to MFI from binding to 47_{WT}.

FIG. 31: Expression of CD47 A30P (47_{A30P}) or Q31P (47_{Q31P}) on T cells leads to loss of B6H12 binding but retained SIRPα binding. (A) Representative flow cytometry histograms of 50 nM hSIRPα, 50 nM mSIRPα, 500 nM B6H12, 500 nM TJC4, and 50 nM Hu5F9 binding to 47_{WT}, 47_{A30P}, 47_{Q31P}, and 47_{A30P-Q31A} over-expressed on primary human T cells. Data are representative of n=3 independent experiments. (B) Binding of B6H12, TJC4, hSIRPα, and mSIRPα to full-length 47_{WT}, 47_{A30P}, and 47_{Q31P} expressed on primary human T cells. Data are the mean±SD of n=3 donors, normalized to fraction binding to 47_{WT}.

FIG. 32: Expression of CD47 A30P (47_{A30P}) or Q31P (47_{Q31P}) on Jurkat cells leads to loss of B6H12 binding but retained SIRPα binding. (A) Representative flow cytometry histograms of 100 nM hSIRPα, 100 nM mSIRPα, and 500 nM B6H12 binding to 47_{WT}, 47_{A30P}, and 47_{Q31P} expressed on Jurkats with endogenous 47_{KO}. Data are representative of n=3 independent experiments. (B) Binding of B6H12, hSIRPα, and mSIRPα to full-length 47_{WT}, 47_{A30P}, and 47_{Q31P} expressed on Jurkat cells with endogenous 47_{KO}. Data are the mean±SD of n=3 independent experiments, normalized to fraction binding to 47_{WT}.

FIG. 33: Expression of CD47 A30P (47_{A30P}) or Q31P (47_{Q31P}) on Jurkat cells abrogates B6H12 mediated phagocytosis by human macrophages. (A and B) Quantification of phagocytosis by primary human macrophages from multiple donors of CFSE labeled Jurkats with endogenous 47_{KO}, expressing 47_{WT}, 47_{A30P}, or 47_{Q31P} variants after one hour of co-culture±B6H12. Data are the mean±SD of n=3 triplicate wells. In (B) the data represent the fold change between untreated and B6H12 treated conditions for each Jurkat-macrophage pairing.

FIG. 34: Expression of engineered CD47 (47_{E}) on T cells abrogates B6H12 mediated phagocytosis by human macrophages. Quantification of phagocytosis by primary human macrophages from two donors of CFSE labeled primary human CD19.28ζ-CAR T cells from three donors with endogenous 47_{KO}, expressing 47_{WT} or 47_{E} (47_{Q31P}) after one hour of co-culture±B6H12. Data are the mean±SD of n=3 triplicate wells.

FIG. 35: Expression of engineered CD47 on T cells prevents T cell depletion in vivo after anti-CD47 treatment. (A) T cell depletion scheme. Non-tumor bearing mice were treated IV with 5×10⁶ 47_{WT}- or 47_{E}-CD19.28ζ-nLuc-CAR T cells (with endogenous 47_{KO}), ±two doses of B6H12 (250 μg/dose; IP) on days 3 and 5 post CAR T treatment. Mice were imaged by BLI before (day 3) and after (day 7) αCD47 treatment, and had blood drawn on day 6 for detection of T cells. (B) Quantification of CD8+ T cells treated as described in (A) in the blood after B6H12 treatment (day 6). Data are the mean±SD of n=5 mice. (C) Quantification of T cell BLI in the 47_{E}-T cell depletion model before and after αCD47 treatment, treated as described in (A), with mice treated IV with 2×10⁶ or 5×10⁶ 47_{WT}- or 47_{E}-CD19.28ζ-nLuc-CAR T cells, as indicated. Data are the mean±SD of n=5 mice. (D) Quantification of CD8⁺ (left) and CD4⁺ (right) T cells in the blood on day 6 by flow cytometry in the 47_{E}-T cell depletion model, treated as described in (A), with mice treated with 2×10⁶ or 5×10⁶ 47_{WT}- or 47_{E}-CD19.28ζ-nLuc-CAR T cells, as indicated. Data are the mean±SD of n=5 mice.

FIG. 36: CAR T cell treatment results in macrophage recruitment into tumors. (A) Scheme of mechanistic study in 143B osteosarcoma. Mice were engrafted orthotopically in the tibia periosteum with 1×10⁶ 143B-CD19 cells and treated IV with no T cells, or 4×10⁶ mock, 47_{WT}- or 47_{E}-Her2.BBζ-CAR T cells (with endogenous 47_{KO}) on day 13. Mice were then treated±two doses of B6H12 (250 μg/dose; IP) on days 15 and 19. Tumors were excised on day 21 and then analyzed via flow cytometry, IHC, and scRNA-seq. (B) Quantification of mF4/80⁺/mCD11 b⁺ macrophages identified by flow cytometry of dissociated tumors treated as described in (A). Data are the mean±SD of n=2 (47_{E}-CAR+B6H12) or n=3 (all other samples) mice. (C) Quantification of mF4/80 staining of IHC sections produced from tumor sections generated as described in (A). Data are the mean±SD of n=3 mice. (D) Human CD45⁺ T cells identified by flow cytometry of dissociated tumors, treated as described in (A). Data are the mean±SD of n=2 (47_{E}-CAR+B6H12) or n=3 (all other samples) mice. (E) Quantification of hCD3 staining of IHC sections produced from tumor sections, treated as described in (A). Data are the mean±SD of n=3 mice.

FIG. 37: CAR T cell tumor infiltration correlates with macrophage recruitment into tumors. (A) Correlation of quantification of hCD3 and mF4/80 staining in IHC sections of tumors treated as described in FIG. 36A. Data points are representative of individual tumors, colored by treatment group (n=23). R² calculated by simple linear regression. (B) Quantification of human CD3 (T cells), mouse Arg1 (M2 macrophages), and mouse F4/80 (total macrophages) staining of IHC sections produced from tumor sections, treated as described in FIG. 36A. Data are the mean±SD of n=2-3 mice. (C) Correlation of quantification of hCD3 and mArg1 staining in IHC sections of tumors treated as described in FIG. 36A. Data points are representative of individual tumors, colored by treatment group (n=23). R² calculated by simple linear regression.

FIG. 38: Single cell RNA sequencing identifies multiple immune subtypes in CAR T treated tumors. (A and B) scRNA-seq profile of dissociated tumor and infiltrating immune cells. Dots represent individual cells. n=53,062 cells from 8 experimental conditions with three mice per treatment group, colored by (A) cell type (left eight plots; UMAPs represent distinct treatment conditions), species (far right), or (B) gene expression level.

FIG. 39: Single cell RNA sequencing confirms CAR T cell treatment results in macrophage recruitment and differential gene expression in tumors treated with 47_{E}-CAR T and anti-CD47. (A) Composition of (left) all cell types or (right) solely mouse cells identified via scRNA-seq from tumors treated as described in FIG. 36A. Data are derived from n=53,062 cells pooled from 8 experimental conditions with three mice per treatment group. Cell types assigned using SingleR automated cell type recognition. (B) Comparison of differentially expressed genes between CAR T cells of different treatment groups described in FIG. 36A. Statistical significance was determined with Seurat; *P adj <0.05. (C) Comparison of differentially expressed genes between macrophages of different treatment groups described in FIG. 36A. Statistical significance was determined with Seurat; *P adj <0.05.

FIG. 40: Single cell RNA sequencing reveals expression of distinct gene pathways after treatment with 47_{E}-CAR T. (A) Dot Plot depicting scRNA-seq expression of selected T cell subset markers, cytokines, and chemokines. n=11,044 human tumor infiltrating T cells from 4 experimental conditions described in FIG. 36A. Genes encoding proteins involved in macrophage “M1” or “M2” polarization are indicated by purple or blue plus signs, respectively. Genes encoding proteins that are non-species cross reactive between human and mouse are marked with a red “x.” (B) Enrichr pathway analysis of the top 100 upregulated genes in tumor infiltrating CAR T cells in 47_{E}-CAR+B6H12 treated tumors compared with 47_{WT}-CAR+B6H12 treated tumors. The NCI-Nature 2016 collection was queried with gene IDs. (C) Enrichr pathway analysis of the top 100 upregulated genes in tumor infiltrating macrophages [monocyte/macrophage cluster in (FIG. 38A)] in 47_{E}-CAR+B6H12 treated tumors compared with untreated controls. The KEGG Human Pathway collection was queried with converted murine gene IDs.

FIG. 41: CAR T recruitment of macrophages leads to tumor infiltration of new populations of macrophages, maintained upon 47_{E}-CAR T and anti-CD47 co-treatment. (A) UMAPs of the identified macrophage/monocyte population [FIG. 38], subsetted and re-clustered, colored by cluster. UMAPs represent distinct treatment conditions. Dots represent individual cells. n=13,082 cells from 8 experimental conditions described in FIG. 36A. (B) UMAP of the re-clustered macrophage/monocyte population from (A), colored for red if hCD3E mRNA expression >0. Dots represent individual cells. n=13,082 cells from 8 experimental conditions, with n=350 total cells identified as hCD3ε mRNA*. UMAPs represent distinct treatment conditions. (C) Composition of macrophage clusters identified in (A) across experimental conditions described in FIG. 36A. (D) Dot plot depicting scRNA-seq expression of selected cluster-defining genes within the macrophage populations identified in (A). P adj. <0.0001 for each selected gene

FIG. 42: Expression of engineered CD47 on CAR T cells permits pairing with anti-CD47 therapy in a 143B osteosarcoma model. (A) 143B treatment scheme. Mice were engrafted orthotopically in the tibia periosteum with 1 λ10⁶ 143B cells and treated IV with 4×10⁶ mock-Antares, or 47_{WT}- or 47_{E}-Her2.BBζ-Antares-CAR T cells (with endogenous 47_{KO}) on day 5. Mice were then treated±two doses of B6H12 (250 μg/dose; IP) on days 7 and 11. Mice were imaged by BLI before (day 7) and after (day 13) αCD47 treatment, and had blood drawn on day 14. (B) Quantification of T cells in the blood (day 14) and by (c) BLI (day 13) in the 143B tumor model treated as described in (A). Data are the mean±SD of n=5 mice. (C) Quantification of T cell BLI prior to B6H12 treatment in the 143B model, treated as described in (A). Data are the mean±SD of n=5 mice. (D) Quantification of T cell BLI (day 13) in the 143B tumor model treated as described in (A). Data are the mean±SD of n=5 mice.

FIG. 43: Expression of engineered CD47 on CAR T cells permits pairing with anti-CD47 therapy and results in improved tumor control in a 143B osteosarcoma model. (A and B) 143B tumor (A) growth and (B) survival, treated as described in FIG. 42A. Data are (A) the mean±SEM or (B) representative of n=5 mice/arm.

FIG. 44: Expression of engineered CD47 on CAR T cells permits pairing with anti-CD47 therapy and results in improved tumor control in a 143B osteosarcoma model after treatment with T cells derived from a second T cell donor. (A and B) 143B tumor (A) growth and (B) survival, treated as described in FIG. 42A with a different T cell donor than FIG. 43. Data are (A) the mean±SEM or (B) representative of n=5 mice/arm.

FIG. 45: Treatment with low doses of anti-CD47 leads to T cell depletion but is prevented by 47_{E} expression. Quantification of CD8⁺ (top) and CD4⁺ (bottom) human T cells derived from in the blood on day 12 by flow cytometry after low-dose B6H12 treatment in the 143B model. Mice were engrafted orthotopically in the tibia periosteum with 0.5×10⁶ 143B cells and treated IV with 4×10⁶ 47_{WT}- or 47_{E}-Her2.BBζ-CAR T cells (with endogenous 47_{KO}) on day 5. Mice were then treated±two doses of B6H12 (75 μg [~3 mg/kg] or 25 μg [~1 mg/kg] per dose; IP) on days 6 and 10. Blood was drawn on day 12. Data are the mean±SD of n=5 mice.

FIG. 46: 47_{E}-CAR T paired with low-dose anti-CD47 therapy results in improved tumor control in a 143B osteosarcoma model. (A) Low dose αCD47 vs 143B treatment scheme. Mice were engrafted orthotopically in the tibia periosteum with 0.5×10⁶ 143B cells and treated IV with 4×10⁶ mock or 47_{E}-Her2.BBζ-CAR T cells (with endogenous 47_{KO}) on day 5. Mice were then treated±two doses of B6H12 (75 μg [−3 mg/kg] or 25 μg [~1 mg/kg] per dose; IP) on days 6 and 10. (B) 143B tumor growth the low-dose αCD47-143B model, treated as described in (A), using T cells derived from two different donors (top and bottom panels, respectively). Mice treated with mock T cells were co-treated±250 μg [~10 mg/kg]B6H12 (top panel) or 75 μg [~3 mg/kg]B6H12 (bottom panel). 47_{E}-Her2.BBζ-CAR T treated mice were co-treated with B6H12 at the doses indicated, as described in (A). Data are the mean±SEM of n=5 mice.

FIG. 47: Expression of engineered CD47 on CAR T cells permits pairing with anti-CD47 therapy in a CHLA-255 metastatic neuroblastoma model. (A) Schematic of CHLA-255 metastatic neuroblastoma model treatment. Mice were engrafted IV with 1×10⁶ CHLA-255-fLuc cells and treated IV with 2×10⁶ mock-nLuc, 47_{WT}- or 47_{E}-B7H3.BBζ-nLuc-CAR T cells (with endogenous 47_{KO}) on day 7. Mice were then treated±three doses of B6H12 (250 μg/dose; IP) on days 7, 9 and 13. T cells were imaged by BLI on day 14 and blood was collected on day 15. (B) Quantification of hCD8⁺ (left) and hCD4⁺ (right) T cells by flow cytometry from the blood of CAR T treated mice on day 15 in the CHLA-255 model, treated as described in (A). Data are the mean±SD of n=5 mice.

FIG. 48: 47_{E}-CAR T paired with anti-CD47 therapy results in improved tumor control in a CHLA-255 metastatic neuroblastoma model. (A) Quantification of T cell BLI on day 14 in the CHLA-255 model, treated as described in FIG. 47A. Data are the mean±SD of n=5 mice. (B) Quantification of CHLA-255 tumor growth by BLI, treated as described in FIG. 47A. Data are the mean±SEM of n=5 mice/arm.

FIG. 49: Expression of engineered CD47 on CAR T cells permits pairing with anti-CD47 therapy and results in improved tumor control in a Nalm6 leukemia model. (A) 47_{E}-CAR T-Nalm6 model treatment scheme. Mice engrafted IV with 1×10⁶ Nalm6-fLuc cells were then treated IV with 0.15×10⁶ mock or 47_{WT}- or 47_{E}-CD19.28ζ-CAR T cells on day 4. Mice were then treated±B6H12 (250 μg/dose; IP) on days 5 and 7. Mice were serially imaged by BLI for tumor growth. (B) Quantification of Nalm6 tumor growth by BLI treated as described in (A). Data are the mean±SEM of n=5 mice/arm for tumor growth. (C) Survival of Nalm6 tumor bearing mice treated as described in (A). n=5 mice per treatment arm.

FIG. 50: Expression of engineered CD47 on TCR T cells permits pairing with anti-CD47 therapy. (A) Schematic of quantification of T cells in the A375-NY-ESO-1 model. Mice engrafted SQ with 3×10⁶ A375 were treated IV with 2.75×10⁶ mock-Antares or 47_{E}-NY-ESO-1-Antares-TCR T cells (with endogenous 47_{KO}) on day 7±three doses of B6H12 (250 μg/dose; IP) on days 9, 11, and 14. Mice were imaged by BLI before (day 9) and after (day 14) αCD47 treatment. Blood was collected on day 15. (B) Quantification of T cells by BLI in the A375-NY-ESO-1 model before anti-CD47 treatment (day 9), treated as described in (A). Data are the mean±SD of n=5 mice. (C and D) Quantification of T cells by (C) BLI and (D) in the blood (left: hCD45⁺; middle: hCD4⁺; right: hCD8⁺) in the A375-NY-ESO-1 model of mice treated as described in (A). Mice were imaged by BLI before (day 9) and after (day 14) anti-CD47 treatment. Blood was collected on day 15. Data are the mean±SD of n=5 mice.

FIG. 51: 47_{E}-TCR T paired with anti-CD47 therapy results in improved tumor control in an A375 melanoma model. (A) A375 treatment scheme with NY-ESO-1-TCR T cells. Mice were engrafted SQ with 3×10⁶ A375 cells and treated IV with 1×10⁶ mock-Antares or 47_{E}-NY-ESO-1-Antares-TCR T cells (with endogenous 47_{KO}) on day 14. Mice were then treated±two doses of B6H12 (250 μg/dose; IP) on days 15 and 19. (B) A375 tumor growth treated as described in (A). Data are the mean±SEM of n=5 mice/arm. (C) Quantification of A375 tumor growth. Mice engrafted SQ with 3×10⁶ A375 were treated IV with 1×10⁶ mock or 47_{E}-NY-ESO-1-TCR T cells (with endogenous 47_{KO}) on day 7±three doses of B6H12 (250 μg/dose; IP) on days 9, 11, and 14, with T cells derived from a different donor than shown in (B). Data are the mean±SEM of n=5 mice/arm. (D) Individual A375 tumor growth traces of mice depicted in (B), treated as described in (A).

### DETAILED DESCRIPTION

Before the nucleic acids, CD47 polypeptides, cells, compositions and methods of the present disclosure are described in greater detail, it is to be understood that the nucleic acids, CD47 polypeptides, cells, compositions and methods are not limited to particular embodiments described, as such may, of course, vary. It is also to be understood that the terminology used herein is for the purpose of describing particular embodiments only, and is not intended to be limiting, since the scope of the nucleic acids, CD47 polypeptides, cells, compositions and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the nucleic acids, CD47 polypeptides, cells, compositions and methods. The upper and lower limits of these smaller ranges may independently be included in the smaller ranges and are also encompassed within the nucleic acids, CD47 polypeptides, cells, compositions and methods, subject to any specifically excluded limit in the stated range. Where the stated range includes one or both of the limits, ranges excluding either or both of those included limits are also included in the nucleic acids, CD47 polypeptides, cells, compositions and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the nucleic acids, CD47 polypeptides, cells, compositions and methods belong. Although any nucleic acids, CD47 polypeptides, cells, compositions and methods similar or equivalent to those described herein can also be used in the practice or testing of the nucleic acids, CD47 polypeptides, cells, compositions and methods, representative illustrative nucleic acids, CD47 polypeptides, cells, compositions and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present nucleic acids, CD47 polypeptides, cells, compositions and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the nucleic acids, CD47 polypeptides, cells, compositions and methods, which are, for clarity, described in the context of separate embodiments, may also be provided in combination in a single embodiment. Conversely, various features of the nucleic acids, CD47 polypeptides, cells, compositions and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present nucleic acids, CD47 polypeptides, cells, compositions and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Nucleic Acids and Cd47 Polypeptides

Aspects of the present disclosure include nucleic acids encoding CD47 polypeptides. CD47 is a widely expressed transmembrane protein with numerous functions. CD47 functions as a ligand for signal regulatory protein-α (SIRPα), a protein expressed on macrophages and dendritic cells. Upon binding CD47, SIRPα initiates a signaling cascade that results in the inhibition of phagocytosis. This “don't eat me” signal is transmitted by phosphorylation of the immunoreceptor tyrosine-based inhibition motifs present on the cytoplasmic tail of SIRPα. Subsequent binding and activation of SHP-1 and SHP-2 (src homology-2 (SH2)-domain containing protein tyrosine phosphatases) blocks phagocytosis.

Blockade of the CD47/SIRPα axis is an area of ongoing therapeutic research, with numerous agents in clinical trials. As demonstrated herein, pairing of adoptive immune cell (e.g., CAR T cell) therapy and anti-CD47 therapy leads to loss of adoptive immune cell anti-tumor efficacy in vivo due to adoptive immune cell depletion. Surprisingly, the nucleic acids and CD47 polypeptides of the present disclosure address the current deficiencies of pairing these therapies by enabling the adoptive immune cells expressing the engineered (mutant) CD47 polypeptide to escape the anti-CD47 therapy/blockade while still retaining binding to SIRPα, in turn preventing macrophage mediated phagocytosis of the adoptive immune cells. Further demonstrated herein is that the engineered CD47 polypeptides surprisingly allow for enhanced anti-tumor efficacy through paired immunotherapy (adoptive immune cell therapy paired with anti-CD47 therapy) even at low doses of the adoptive immune cells and low doses of anti-CD47 therapy. Strikingly, the engineered CD47 polypeptides enable profound anti-tumor efficacy even for cancers where both the adoptive immune cell therapy and the anti-CD47 therapy have minimal effect as monotherapies. Moreover, demonstrated herein is that treatment using adoptive immune cells expressing the engineered CD47 polypeptides unexpectedly leads to macrophage tumor infiltration and potentiates the efficacy of CD47 blockade. Embodiments of the nucleic acids and CD47 polypeptides of the present disclosure will now be described in further detail.

The terms “nucleic acid” and “polynucleotide” are used interchangeably herein to describe a polymer of any length composed of nucleotides, e.g., deoxyribonucleotides, and may be produced enzymatically or synthetically. Naturally-occurring nucleotides include guanine, cytosine, adenine, thymine, uracil (G, C, A, T and U respectively). DNA and RNA have a deoxyribose and ribose sugar backbone, respectively. The terms “polypeptide” and “protein” are used interchangeably herein to designate a linear series of amino acid residues connected one to the other by peptide bonds between the alpha-amino and carboxy groups of adjacent residues. The amino acids may include the 20 “standard” genetically encodable amino acids, amino acid analogs, or a combination thereof.

In certain embodiments, the nucleic acids of the present disclosure encode a CD47 polypeptide comprising a mutant CD47 Ig-like domain that reduces binding of a therapeutic anti-CD47 binding agent to the CD47 polypeptide as compared to binding of the therapeutic anti-CD47 antibody to a wild-type CD47 polypeptide, and wherein the CD47 polypeptide retains binding to SIRPα. In certain embodiments, the mutant CD47 Ig-like domain comprises a mutant BC loop.

By “mutant” Ig-like domain or “mutant” BC loop is meant the Ig-like domain (e.g., BC loop) includes one or more amino acid substitutions, insertions, deletions, or any combination thereof, which reduce binding of the therapeutic anti-CD47 binding agent to the CD47 polypeptide while retaining binding to SIRPα. Such mutations may be introduced using a variety of available genetic engineering and mutagenesis (e.g., site-directed mutagenesis (“SDM”)) techniques known in the art, including PCR-based approaches, non-PCR-based approaches (e.g., CRISPR-Cas9-based approaches, TALEN-based approaches, Zinc Finger Nuclease (ZFN)-based approaches), etc.

The amino acid sequences of the wild-type CD47 Ig-like domain and wild-type BC loop of human CD47 are provided in SEQ ID NO:1 in Table 1 below, where amino acids 1-117 constitute the wild-type Ig-like domain and amino acids 26-31 constitute the wild-type BC loop. The Ig-like domains are underlined in the amino acid sequences of Table 1. Not shown in the amino acid sequences in Table 1 is an N-terminal signal sequence, which may be included in, and encoded by, any of the CD47 polypeptides and nucleic acids (respectively) of the present disclosure. An exemplary N-terminal signal sequence is MWPLVAALLLGSACCGSA (SEQ ID NO:16). The nucleic acid sequences in Table 1 encode the N-terminal signal sequence of SEQ ID NO:16.

According to some embodiments, a nucleic acid of the present disclosure encodes a CD47 polypeptide comprising a mutant BC loop, where the mutant BC loop comprises an amino acid substitution at E29, A30, Q31, or any combination thereof. Numbering is according to the amino acid sequence set forth in SEQ ID NO:1 in Table 1. In certain embodiments, the mutant BC loop comprises the amino acid substitution E29A, A30P, Q31P/Q31A (i.e., Q31P or Q31A), or any combination thereof.

TABLE 1
Amino Acid and Nucleic Acid Sequences of Wild-Type
and Exemplary Engineered CD47
Polypeptides and Nucleic Acids Encoding Same

| Polypeptide or | | SEQ ID |
|---|---|---|
| Nucleic Acid | Sequence | NO: |
| Human CD47 | QLLFNKTKSVEFTFCNDTVVIPCFVTNMEAQNTTEVYVKWKFKG | 1 |
| wild-type | RDIYTFDGALNKSTVPTDFSSAKIEVSQLLKGDASLKMDKSDAV | |
| amino acid | SHTGNYTCEVTELTREGETIIELKYRVVSWFSPNENILIVIFPIFAIL | |
| sequence | LFWGQFGIKTLKYRSGGMDEKTIALLVAGLVITVIVIVGAILFVPG | |
| | EYSLKNATGLGLIVTSTGILILLHYYVFSTAIGLTSFVIAILVIQVIAYI | |
| | LAVVGLSLCIAACIPMHGPLLISGLSILALAQLLGLVYMKFVASNQ | |
| | KTIQPPRKAVEEPLNAFKESKGMMNDE | |
| Human CD47 | QLLFNKTKSVEFTFCNDTVVIPCFVTNMEPQNTTEVYVKWKFKG | 2 |
| (A30P) amino | RDIYTFDGALNKSTVPTDFSSAKIEVSQLLKGDASLKMDKSDAV | |
| acid sequence | SHTGNYTCEVTELTREGETIIELKYRVVSWFSPNENILIVIFPIFAIL | |
| | LFWGQFGIKTLKYRSGGMDEKTIALLVAGLVITVIVIVGAILFVPG | |
| | EYSLKNATGLGLIVTSTGILILLHYYVFSTAIGLTSFVIAILVIQVIAYI | |
| | LAVVGLSLCIAACIPMHGPLLISGLSILALAQLLGLVYMKFVASNQ | |
| | KTIQPPRKAVEEPLNAFKESKGMMNDE | |
| Human CD47 | ATGTGGCCGTTGGTTGCAGCACTCTTGCTTGGATCTGCTTGC | 3 |
| (A30P) nucleic | TGTGGATCCGCTCAACTCCTTTTTAATAAGACGAAATCCGTC | |
| acid sequence | GAATTTACATTTTGCAATGATACCGTCGTTATCCCATGCTTTG | |
| | TCACGAACATGGAGCCCCAGAATACGACCGAAGTCTACGTG | |
| | AAGTGGAAATTTAAGGGACGCGATATTTATACTTTCGATGGG | |
| | GCATTGAACAAATCCACAGTCCCAACCGACTTTTCCTCCGCG | |
| | AAAATCGAAGTTAGTCAACTTCTTAAAGGAGACGCGAGTCTC | |
| | AAGATGGACAAATCCGACGCCGTGAGTCATACTGGTAATTAC | |
| | ACCTGCGAGGTCACGGAACTGACACGAGAAGGGGAGACTAT | |
| | AATAGAACTTAAATACCGAGTCGTTTCCTGGTTTAGTCCGAAT | |
| | GAGAATATTCTGATTGTTATATTTCCGATATTTGCCATCCTGT | |
| | TGTTCTGGGGCCAATTCGGAATAAAGACTCTCAAGTATCGCA | |
| | GCGGGGGAATGGACGAAAAAACCATTGCTCTCCTGGTGGCC | |
| | GGTCTTGTAATTACTGTGATAGTGATTGTTGGCGCAATTCTCT | |
| | TTGTGCCAGGGGAATATTCTCTCAAGAATGCCACTGGATTGG | |
| | GTTTGATAGTTACCTCCACGGGCATACTGATCCTTCTTCATTA | |
| | CTATGTATTTAGCACAGCCATTGGTCTTACAAGCTTTGTTATT | |
| | GCCATCCTGGTAATTCAGGTAATAGCATACATCCTCGCAGTC | |
| | GTTGGACTTAGCTTGTGCATAGCAGCTTGCATACCTATGCAC | |
| | GGTCCACTGTTGATCTCAGGGTTGTCCATACTCGCACTCGCG | |
| | CAGCTGCTTGGTCTCGTGTACATGAAATTCGTAGCATCAAAC | |
| | CAAAAAACCATACAACCGCCCAGGAAGGCTGTGGAAGAACC | |
| | CCTGAACGCATTTAAGGAATCCAAGGGGATGATGAACGATGA | |
| | G | |
| Human CD47 | QLLFNKTKSVEFTFCNDTVVIPCFVTNMEAPNTTEVYVKWKFKG | 4 |
| (Q31P) amino | RDIYTFDGALNKSTVPTDFSSAKIEVSQLLKGDASLKMDKSDAV | |
| acid sequence | SHTGNYTCEVTELTREGETIIELKYRVVSWFSPNENILIVIFPIFAIL | |
| | LFWGQFGIKTLKYRSGGMDEKTIALLVAGLVITVIVIVGAILFVPG | |
| | EYSLKNATGLGLIVTSTGILILLHYYVFSTAIGLTSFVIAILVIQVIAYI | |
| | LAVVGLSLCIAACIPMHGPLLISGLSILALAQLLGLVYMKFVASNQ | |
| | KTIQPPRKAVEEPLNAFKESKGMMNDE | |
| Human CD47 | ATGTGGCCGTTGGTTGCAGCACTCTTGCTTGGATCTGCTTGC | 5 |
| (Q31P) nucleic | TGTGGATCCGCTCAACTCCTTTTTAATAAGACGAAATCCGTC | |
| acid sequence | GAATTTACATTTTGCAATGATACCGTCGTTATCCCATGCTTTG | |
| | TCACGAACATGGAGGCCCCGAATACGACCGAAGTCTACGTG | |
| | AAGTGGAAATTTAAGGGACGCGATATTTATACTTTCGATGGG | |
| | GCATTGAACAAATCCACAGTCCCAACCGACTTTTCCTCCGCG | |
| | AAAATCGAAGTTAGTCAACTTCTTAAAGGAGACGCGAGTCTC | |
| | AAGATGGACAAATCCGACGCCGTGAGTCATACTGGTAATTAC | |
| | ACCTGCGAGGTCACGGAACTGACACGAGAAGGGGAGACTAT | |
| | AATAGAACTTAAATACCGAGTCGTTTCCTGGTTTAGTCCGAAT | |
| | GAGAATATTCTGATTGTTATATTTCCGATATTTGCCATCCTGT | |
| | TGTTCTGGGGCCAATTCGGAATAAAGACTCTCAAGTATCGCA | |
| | GCGGGGGAATGGACGAAAAAACCATTGCTCTCCTGGTGGCC | |
| | GGTCTTGTAATTACTGTGATAGTGATTGTTGGCGCAATTCTCT | |
| | TTGTGCCAGGGGAATATTCTCTCAAGAATGCCACTGGATTGG | |
| | GTTTGATAGTTACCTCCACGGGCATACTGATCCTTCTTCATTA | |
| | CTATGTATTTAGCACAGCCATTGGTCTTACAAGCTTTGTTATT | |
| | GCCATCCTGGTAATTCAGGTAATAGCATACATCCTCGCAGTC | |
| | GTTGGACTTAGCTTGTGCATAGCAGCTTGCATACCTATGCAC | |
| | GGTCCACTGTTGATCTCAGGGTTGTCCATACTCGCACTCGCG | |
| | CAGCTGCTTGGTCTCGTGTACATGAAATTCGTAGCATCAAAC | |
| | CAAAAAACCATACAACCGCCCAGGAAGGCTGTGGAAGAACC | |
| | CCTGAACGCATTTAAGGAATCCAAGGGGATGATGAACGATGA | |
| | G | |
| Human CD47 | QLLFNKTKSVEFTFCNDTVVIPCFVTNMAAQNTTEVYVKWKFK | 6 |
| (E29A) amino | GRDIYTFDGALNKSTVPTDFSSAKIEVSQLLKGDASLKMDKSDA | |
| acid sequence | VSHTGNYTCEVTELTREGETIIELKYRVVSWFSPNENILIVIFPIFA | |
| | ILLFWGQFGIKTLKYRSGGMDEKTIALLVAGLVITVIVIVGAILFVP | |
| | GEYSLKNATGLGLIVTSTGILILLHYYVFSTAIGLTSFVIAILVIQVIA | |
| | YILAVVGLSLCIAACIPMHGPLLISGLSILALAQLLGLVYMKFVASN | |
| | QKTIQPPRKAVEEPLNAFKESKGMMNDE | |
| Human CD47 | ATGTGGCCGTTGGTTGCAGCACTCTTGCTTGGATCTGCTTGC | 7 |
| (E29A) nucleic | TGTGGATCCGCTCAACTCCTTTTTAATAAGACGAAATCCGTC | |
| acid sequence | GAATTTACATTTTGCAATGATACCGTCGTTATCCCATGCTTTG | |
| | TCACGAACATGGCAGCCCAGAATACGACCGAAGTCTACGTG | |
| | AAGTGGAAATTTAAGGGACGCGATATTTATACTTTCGATGGG | |
| | GCATTGAACAAATCCACAGTCCCAACCGACTTTTCCTCCGCG | |
| | AAAATCGAAGTTAGTCAACTTCTTAAAGGAGACGCGAGTCTC | |
| | AAGATGGACAAATCCGACGCCGTGAGTCATACTGGTAATTAC | |
| | ACCTGCGAGGTCACGGAACTGACACGAGAAGGGGAGACTAT | |
| | AATAGAACTTAAATACCGAGTCGTTTCCTGGTTTAGTCCGAAT | |
| | GAGAATATTCTGATTGTTATATTTCCGATATTTGCCATCCTGT | |
| | TGTTCTGGGGCCAATTCGGAATAAAGACTCTCAAGTATCGCA | |
| | GCGGGGGAATGGACGAAAAAACCATTGCTCTCCTGGTGGCC | |
| | GGTCTTGTAATTACTGTGATAGTGATTGTTGGCGCAATTCTCT | |
| | TTGTGCCAGGGGAATATTCTCTCAAGAATGCCACTGGATTGG | |
| | GTTTGATAGTTACCTCCACGGGCATACTGATCCTTCTTCATTA | |
| | CTATGTATTTAGCACAGCCATTGGTCTTACAAGCTTTGTTATT | |
| | GCCATCCTGGTAATTCAGGTAATAGCATACATCCTCGCAGTC | |
| | GTTGGACTTAGCTTGTGCATAGCAGCTTGCATACCTATGCAC | |
| | GGTCCACTGTTGATCTCAGGGTTGTCCATACTCGCACTCGCG | |
| | CAGCTGCTTGGTCTCGTGTACATGAAATTCGTAGCATCAAAC | |
| | CAAAAAACCATACAACCGCCCAGGAAGGCTGTGGAAGAACC | |
| | CCTGAACGCATTTAAGGAATCCAAGGGGATGATGAACGATGA | |
| | G | |
| Human CD47 | QLLFNKTKSVEFTFCNDTVVIPCFVTNMEPANTTEVYVKWKFKG | 8 |
| (A30P-Q31A) | RDIYTFDGALNKSTVPTDFSSAKIEVSQLLKGDASLKMDKSDAV | |
| amino acid | SHTGNYTCEVTELTREGETIIELKYRVVSWFSPNENILIVIFPIFAIL | |
| sequence | LFWGQFGIKTLKYRSGGMDEKTIALLVAGLVITVIVIVGAILFVPG | |
| | EYSLKNATGLGLIVTSTGILILLHYYVFSTAIGLTSFVIAILVIQVIAYI | |
| | LAVVGLSLCIAACIPMHGPLLISGLSILALAQLLGLVYMKFVASNQ | |
| | KTIQPPRKAVEEPLNAFKESKGMMNDE | |
| Human CD47 | ATGTGGCCGTTGGTTGCAGCACTCTTGCTTGGATCTGCTTGC | 9 |
| (A30P-Q31A) | TGTGGATCCGCTCAACTCCTTTTTAATAAGACGAAATCCGTC | |
| nucleic acid | GAATTTACATTTTGCAATGATACCGTCGTTATCCCATGCTTTG | |
| sequence | TCACGAACATGGAGCCCGCGAATACGACCGAAGTCTACGTG | |
| | AAGTGGAAATTTAAGGGACGCGATATTTATACTTTCGATGGG | |
| | GCATTGAACAAATCCACAGTCCCAACCGACTTTTCCTCCGCG | |
| | AAAATCGAAGTTAGTCAACTTCTTAAAGGAGACGCGAGTCTC | |
| | AAGATGGACAAATCCGACGCCGTGAGTCATACTGGTAATTAC | |
| | ACCTGCGAGGTCACGGAACTGACACGAGAAGGGGAGACTAT | |
| | AATAGAACTTAAATACCGAGTCGTTTCCTGGTTTAGTCCGAAT | |
| | GAGAATATTCTGATTGTTATATTTCCGATATTTGCCATCCTGT | |
| | TGTTCTGGGGCCAATTCGGAATAAAGACTCTCAAGTATCGCA | |
| | GCGGGGGAATGGACGAAAAAACCATTGCTCTCCTGGTGGCC | |
| | GGTCTTGTAATTACTGTGATAGTGATTGTTGGCGCAATTCTCT | |
| | TTGTGCCAGGGGAATATTCTCTCAAGAATGCCACTGGATTGG | |
| | GTTTGATAGTTACCTCCACGGGCATACTGATCCTTCTTCATTA | |
| | CTATGTATTTAGCACAGCCATTGGTCTTACAAGCTTTGTTATT | |
| | GCCATCCTGGTAATTCAGGTAATAGCATACATCCTCGCAGTC | |
| | GTTGGACTTAGCTTGTGCATAGCAGCTTGCATACCTATGCAC | |
| | GGTCCACTGTTGATCTCAGGGTTGTCCATACTCGCACTCGCG | |
| | CAGCTGCTTGGTCTCGTGTACATGAAATTCGTAGCATCAAAC | |
| | CAAAAAACCATACAACCGCCCAGGAAGGCTGTGGAAGAACC | |
| | CCTGAACGCATTTAAGGAATCCAAGGGGATGATGAACGATGA | |
| | G | |
| Human CD47 | QLLFNKTKSVEFTFCNDTVVIPCFVTNMAPANTTEVYVKWKFKG | 10 |
| (E29A-A30P- | RDIYTFDGALNKSTVPTDFSSAKIEVSQLLKGDASLKMDKSDAV | |
| Q31A) amino | SHTGNYTCEVTELTREGETIIELKYRVVSWFSPNENILIVIFPIFAIL | |
| acid sequence | LFWGQFGIKTLKYRSGGMDEKTIALLVAGLVITVIVIVGAILFVPG | |
| | EYSLKNATGLGLIVTSTGILILLHYYVFSTAIGLTSFVIAILVIQVIAYI | |
| | LAVVGLSLCIAACIPMHGPLLISGLSILALAQLLGLVYMKFVASNQ | |
| | KTIQPPRKAVEEPLNAFKESKGMMNDE | |
| Human CD47 | ATGTGGCCGTTGGTTGCAGCACTCTTGCTTGGATCTGCTTGC | 11 |
| (E29A-A30P- | TGTGGATCCGCTCAACTCCTTTTTAATAAGACGAAATCCGTC | |
| Q31A) nucleic | GAATTTACATTTTGCAATGATACCGTCGTTATCCCATGCTTTG | |
| acid sequence | TCACGAACATGGCACCCGCGAATACGACCGAAGTCTACGTG | |
| | AAGTGGAAATTTAAGGGACGCGATATTTATACTTTCGATGGG | |
| | GCATTGAACAAATCCACAGTCCCAACCGACTTTTCCTCCGCG | |
| | AAAATCGAAGTTAGTCAACTTCTTAAAGGAGACGCGAGTCTC | |
| | AAGATGGACAAATCCGACGCCGTGAGTCATACTGGTAATTAC | |
| | ACCTGCGAGGTCACGGAACTGACACGAGAAGGGGAGACTAT | |
| | AATAGAACTTAAATACCGAGTCGTTTCCTGGTTTAGTCCGAAT | |
| | GAGAATATTCTGATTGTTATATTTCCGATATTTGCCATCCTGT | |
| | TGTTCTGGGGCCAATTCGGAATAAAGACTCTCAAGTATCGCA | |
| | GCGGGGGAATGGACGAAAAAACCATTGCTCTCCTGGTGGCC | |
| | GGTCTTGTAATTACTGTGATAGTGATTGTTGGCGCAATTCTCT | |
| | TTGTGCCAGGGGAATATTCTCTCAAGAATGCCACTGGATTGG | |
| | GTTTGATAGTTACCTCCACGGGCATACTGATCCTTCTTCATTA | |
| | CTATGTATTTAGCACAGCCATTGGTCTTACAAGCTTTGTTATT | |
| | GCCATCCTGGTAATTCAGGTAATAGCATACATCCTCGCAGTC | |
| | GTTGGACTTAGCTTGTGCATAGCAGCTTGCATACCTATGCAC | |
| | GGTCCACTGTTGATCTCAGGGTTGTCCATACTCGCACTCGCG | |
| | CAGCTGCTTGGTCTCGTGTACATGAAATTCGTAGCATCAAAC | |
| | CAAAAAACCATACAACCGCCCAGGAAGGCTGTGGAAGAACC | |
| | CCTGAACGCATTTAAGGAATCCAAGGGGATGATGAACGATGA | |
| | G | |
| Human CD47 | QLLFNKTKSVEFTFCNDTVVIPCFVTNMAPQNTTEVYVKWKFK | 12 |
| (E29A-A30P) | GRDIYTFDGALNKSTVPTDFSSAKIEVSQLLKGDASLKMDKSDA | |
| amino acid | VSHTGNYTCEVTELTREGETIIELKYRVVSWFSPNENILIVIFPIFA | |
| sequence | ILLFWGQFGIKTLKYRSGGMDEKTIALLVAGLVITVIVIVGAILFVP | |
| | GEYSLKNATGLGLIVTSTGILILLHYYVFSTAIGLTSFVIAILVIQVIA | |
| | YILAVVGLSLCIAACIPMHGPLLISGLSILALAQLLGLVYMKFVASN | |
| | QKTIQPPRKAVEEPLNAFKESKGMMNDE | |
| Human CD47 | ATGTGGCCGTTGGTTGCAGCACTCTTGCTTGGATCTGCTTGC | 13 |
| (E29A-A30P) | TGTGGATCCGCTCAACTCCTTTTTAATAAGACGAAATCCGTC | |
| nucleic acid | GAATTTACATTTTGCAATGATACCGTCGTTATCCCATGCTTTG | |
| sequence | TCACGAACATGGCACCCCAGAATACGACCGAAGTCTACGTG | |
| | AAGTGGAAATTTAAGGGACGCGATATTTATACTTTCGATGGG | |
| | GCATTGAACAAATCCACAGTCCCAACCGACTTTTCCTCCGCG | |
| | AAAATCGAAGTTAGTCAACTTCTTAAAGGAGACGCGAGTCTC | |
| | AAGATGGACAAATCCGACGCCGTGAGTCATACTGGTAATTAC | |
| | ACCTGCGAGGTCACGGAACTGACACGAGAAGGGGAGACTAT | |
| | AATAGAACTTAAATACCGAGTCGTTTCCTGGTTTAGTCCGAAT | |
| | GAGAATATTCTGATTGTTATATTTCCGATATTTGCCATCCTGT | |
| | TGTTCTGGGGCCAATTCGGAATAAAGACTCTCAAGTATCGCA | |
| | GCGGGGGAATGGACGAAAAAACCATTGCTCTCCTGGTGGCC | |
| | GGTCTTGTAATTACTGTGATAGTGATTGTTGGCGCAATTCTCT | |
| | TTGTGCCAGGGGAATATTCTCTCAAGAATGCCACTGGATTGG | |
| | GTTTGATAGTTACCTCCACGGGCATACTGATCCTTCTTCATTA | |
| | CTATGTATTTAGCACAGCCATTGGTCTTACAAGCTTTGTTATT | |
| | GCCATCCTGGTAATTCAGGTAATAGCATACATCCTCGCAGTC | |
| | GTTGGACTTAGCTTGTGCATAGCAGCTTGCATACCTATGCAC | |
| | GGTCCACTGTTGATCTCAGGGTTGTCCATACTCGCACTCGCG | |
| | CAGCTGCTTGGTCTCGTGTACATGAAATTCGTAGCATCAAAC | |
| | CAAAAAACCATACAACCGCCCAGGAAGGCTGTGGAAGAACC | |
| | CCTGAACGCATTTAAGGAATCCAAGGGGATGATGAACGATGA | |
| | G | |
| Human CD47 | QLLFNKTKSVEFTFCNDTVVIPCFVTNMAAPNTTEVYVKWKFKG | 14 |
| (E29A-Q31P) | RDIYTFDGALNKSTVPTDFSSAKIEVSQLLKGDASLKMDKSDAV | |
| amino acid | SHTGNYTCEVTELTREGETIIELKYRVVSWFSPNENILIVIFPIFAIL | |
| sequence | LFWGQFGIKTLKYRSGGMDEKTIALLVAGLVITVIVIVGAILFVPG | |
| | EYSLKNATGLGLIVTSTGILILLHYYVFSTAIGLTSFVIAILVIQVIAYI | |
| | LAVVGLSLCIAACIPMHGPLLISGLSILALAQLLGLVYMKFVASNQ | |
| | KTIQPPRKAVEEPLNAFKESKGMMNDE | |
| Human CD47 | ATGTGGCCGTTGGTTGCAGCACTCTTGCTTGGATCTGCTTGC | 15 |
| (E29A-Q31P) | TGTGGATCCGCTCAACTCCTTTTTAATAAGACGAAATCCGTC | |
| nucleic acid | GAATTTACATTTTGCAATGATACCGTCGTTATCCCATGCTTTG | |
| sequence | TCACGAACATGGCAGCCCCGAATACGACCGAAGTCTACGTG | |
| | AAGTGGAAATTTAAGGGACGCGATATTTATACTTTCGATGGG | |
| | GCATTGAACAAATCCACAGTCCCAACCGACTTTTCCTCCGCG | |
| | AAAATCGAAGTTAGTCAACTTCTTAAAGGAGACGCGAGTCTC | |
| | AAGATGGACAAATCCGACGCCGTGAGTCATACTGGTAATTAC | |
| | ACCTGCGAGGTCACGGAACTGACACGAGAAGGGGAGACTAT | |
| | AATAGAACTTAAATACCGAGTCGTTTCCTGGTTTAGTCCGAAT | |
| | GAGAATATTCTGATTGTTATATTTCCGATATTTGCCATCCTGT | |
| | TGTTCTGGGGCCAATTCGGAATAAAGACTCTCAAGTATCGCA | |
| | GCGGGGGAATGGACGAAAAAACCATTGCTCTCCTGGTGGCC | |
| | GGTCTTGTAATTACTGTGATAGTGATTGTTGGCGCAATTCTCT | |
| | TTGTGCCAGGGGAATATTCTCTCAAGAATGCCACTGGATTGG | |
| | GTTTGATAGTTACCTCCACGGGCATACTGATCCTTCTTCATTA | |
| | CTATGTATTTAGCACAGCCATTGGTCTTACAAGCTTTGTTATT | |
| | GCCATCCTGGTAATTCAGGTAATAGCATACATCCTCGCAGTC | |
| | GTTGGACTTAGCTTGTGCATAGCAGCTTGCATACCTATGCAC | |
| | GGTCCACTGTTGATCTCAGGGTTGTCCATACTCGCACTCGCG | |
| | CAGCTGCTTGGTCTCGTGTACATGAAATTCGTAGCATCAAAC | |
| | CAAAAAACCATACAACCGCCCAGGAAGGCTGTGGAAGAACC | |
| | CCTGAACGCATTTAAGGAATCCAAGGGGATGATGAACGATGA | |
| | G | |

Because of the knowledge of the codons corresponding to the various amino acids, availability of an amino acid sequence of a polypeptide of interest provides a description of all the polynucleotides capable of encoding the polypeptide of interest. The degeneracy of the genetic code, where the same amino acids are encoded by alternative or synonymous codons allows an extremely large number of nucleic acids to be made, all of which encode the CD47 polypeptides disclosed herein. Thus, having identified a particular amino acid sequence, those skilled in the art could make any number of different nucleic acids by simply modifying the sequence of one or more codons in a way which does not change the amino acid sequence of the polypeptide of interest. In this regard, the present disclosure specifically contemplates each and every possible variation of polynucleotides that could be made by selecting combinations based upon the possible codon choices, and all such variations are to be considered specifically disclosed for any polypeptide disclosed herein, including the amino acid sequences presented in Table 1. In certain embodiments, variant CD47 polypeptides having one or more amino acid substitutions relative to any of the amino acid sequences set forth in Table 1 are provided.

Conservative substitutions are shown in the following table under the heading of “preferred substitutions.” More substantial changes are provided in the following table under the heading of “exemplary substitutions,” and as further described below in reference to amino acid side chain classes. Amino acid substitutions may be introduced into a CD47 polypeptide of interest and the products screened for a desired activity, e.g., retained/improved escape from an anti-CD47 therapy/blockade of interest, improved binding to SIRPα, decreased immunogenicity, improved expression in an adoptive immune cell, and/or the like.

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

As described above, the CD47 polypeptide comprises a mutant CD47 Ig-like domain that reduces binding of a therapeutic anti-CD47 binding agent to the CD47 polypeptide as compared to binding of the therapeutic anti-CD47 antibody to a wild-type CD47 polypeptide. The therapeutic anti-CD47 binding agent may vary. Therapeutic anti-CD47 binding agents of interest include those that bind to wild-type CD47 and inhibit or block interaction between the bound CD47 and SIRPα. Therapeutic anti-CD47 binding agents of interest include, but are not limited to, therapeutic anti-CD47 antibodies, soluble SIRPα decoys, and the like. The therapeutic anti-CD47 binding agent may be one approved for anti-CD47 therapy by the United States Food and Drug Administration (FDA) and/or the European Medicines Agency (EMA).

In certain embodiments, the therapeutic anti-CD47 binding agent is a therapeutic anti-CD47 antibody. According to some embodiments, the therapeutic anti-CD47 antibody is a therapeutic anti-CD47 antibody approved for anti-CD47 therapy by the United States FDA and/or the EMA. Non-limiting examples of therapeutic anti-CD47 antibodies for which the CD47 polypeptides may exhibit reduced binding include those having the six complementarity determining regions (CDRs) of lemzoparlimab, antibody B6H12, magrolimab (5F9), AK117, AO-176, CC-90002, DSP107, HX009, IB1188, 1B1322, IMC-002, IMM0306, PF-07257876, SHR-1603, SRF231, STI-6643, TG-1801, TJ011133, or ZL-1201. The variable heavy chain (V_{H}) and variably light chain (V_{L}) amino acid sequences—and CDR amino acid sequences therein—of these and other anti-CD47 antibodies of interest are known and readily accessible. For example, the V_{H}, V_{L}, and CDR sequences of antibody B6H12 were known and disclosed, e.g., in U.S. Pat. No. 9,017,675 B2 (see SEQ ID NOs: 3-8). Also by way of example, the V_{H}, V_{L}, and CDR sequences of antibody TJC4 were known and disclosed, e.g., in WO2021219092A1 (see SEQ ID NOs: 86-92). Further, the V_{H}, V_{L}, and CDR sequences of antibody Hu5F9 were known and disclosed, e.g., in U.S. Pat. No. 9,017,675 B2 (see SEQ ID NOs: 20-25). In addition, the V_{H}, V_{L}, and CDR sequences of antibody TJC4 were known and disclosed, e.g., in Puro et al. (2020) 19(3):835-846 (see FIG. S1). The amino acid sequences of CDRs of exemplary anti-CD47 antibodies are also set forth in the table below.

CDR Amino Acid Sequences
of Exemplary anti-CD47 Antibodies

| | | | | SEQ |
|---|---|---|---|---|
| | | | | ID |
| Antibody | Chain | CDR | Sequence | NO |
| B6H12 | VH | CDR1 | GYGMS | 17 |
| | | CDR2 | TITSGGTYTYYPDSVKG | 18 |
| | | CDR3 | SLAGNAMDY | 19 |
| | VL | CDR1 | RASQTISDYLH | 20 |
| | | CDR2 | FASQSIS | 21 |
| | | CDR3 | QNGHGFPRT | 22 |
| Lemzoparlimab | VH | CDR1 | RAWMN | 23 |
| (TJC4) | | CDR2 | RIKRKTDGETTDYAAPVKG | 24 |
| | | CDR3 | SNRAFDI | 25 |
| | VL | CDR1 | KSSQSVLYAGNNRNYLA | 26 |
| | | CDR2 | QASTRAS | 27 |
| | | CDR3 | QQYYTPPLA | 28 |
| Magrolimab | VH | CDR1 | NYNMH | 29 |
| (Hu5F9) | | CDR2 | TIYPGNDDTSYNQKFKD | 30 |
| | | CDR3 | GGYRAMDY | 31 |
| | VL | CDR1 | RSSQSIVYSNGNTYLG | 32 |
| | | CDR2 | KVSNRFS | 33 |
| | | CDR3 | FQGSHVPYTF | 34 |
| AO-176 | VH | CDR1 | GYTFTNYWIH | 35 |
| | | CDR2 | YTDPRTDYTEYNQKFKD | 36 |
| | | CDR3 | GGRVGLGY | 37 |
| | VL | CDR1 | RSSQNIVQSNGNTYLE | 38 |
| | | CDR2 | KVFHRFS | 39 |
| | | CDR3 | FQGSHVPYT | 40 |

The term “antibody” may include an antibody or immunoglobulin of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the cell surface molecule of the target cell, including, but not limited to single chain Fv (scFv), Fab, (Fab′)₂, (scFv′)₂, and diabodies; chimeric antibodies; monoclonal antibodies, human antibodies, humanized antibodies (e.g., humanized whole antibodies, humanized half antibodies, or humanized antibody fragments, e.g., humanized scFv); and fusion proteins comprising an antigen-binding portion of an antibody and a non-antibody protein. According to some embodiments, the antibody is selected from an IgG, Fv, single chain antibody, scFv, Fab, F(ab′)2, or Fab′. In certain embodiments, the antibody is a nanobody (an antibody fragment consisting of a single monomeric variable antibody domain—also known as a single-domain antibody (sdAb)), a monobody (a synthetic binding protein constructed using a fibronectin type Ill domain (FN3) as a molecular scaffold), or a Bi-specific T-cell engager (BiTE).

An immunoglobulin light or heavy chain variable region (V_{L} and V_{H}, respectively) is composed of a “framework” region (FR) interrupted by three hypervariable regions, also called “complementarity determining regions” or “CDRs”. The extent of the framework region and CDRs have been defined (see, E. Kabat et al., Sequences of proteins of immunological interest, 4th ed. U.S. Dept. Health and Human Services, Public Health Services, Bethesda, MD (1987); and Lefranc et al. IMGT, the international ImMunoGeneTics information System®. Nucl. Acids Res., 2005, 33, D593-D597)). The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen.

An “antibody” thus encompasses a protein having one or more polypeptides that can be genetically encodable, e.g., by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. In some embodiments, an antibody of the present disclosure is an IgG antibody, e.g., an IgG1 antibody, such as a human IgG1 antibody. In some embodiments, the cell expresses an antibody that comprises a human Fc domain.

The term “monoclonal antibody” as used herein refers to an antibody obtained from a population of substantially homogeneous antibodies, i.e., the individual antibodies comprising the population are identical except for possible naturally occurring mutations that may be present in minor amounts. For example, a monoclonal antibody can be an antibody that is derived from a single clone, including any eukaryotic, prokaryotic, yeast or phage clone, or produced via a cell-free expression system, and not the method by which it is produced. A monoclonal antibody composition displays a single binding specificity and affinity for a particular epitope. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. Monoclonal antibodies can be prepared using a wide variety of techniques known in the art including, e.g., but not limited to, hybridoma, recombinant, yeast display technologies, phage display technologies, ribosome display technologies, DNA display technologies, and the like. For example, monoclonal antibodies may be made by the hybridoma method first described by Kohler et al, Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al, Nature 352:624-628 (1991) and Marks et al, J. Mol. Biol. 222:581-597 (1991), for example.

The phrases “specifically binds”, “specific for”, “immunoreactive” and “immunoreactivity”, and “antigen binding specificity”, when referring to an antibody, refer to a binding reaction with an antigen which is highly preferential to the antigen or a fragment thereof, so as to be determinative of the presence of and/or selective for the antigen in the presence of a heterogeneous population of antigens (e.g., proteins and other biologics, e.g., in a sample or in vivo). Thus, under designated assay (e.g., immunoassay) conditions, the specified polypeptides bind to a particular antigen and do not bind in a significant amount to other antigens present in the sample. Specific binding to an antigen under such conditions may require a polypeptide that is selected for its specificity for a particular antigen. For example, a polypeptide (e.g., an antibody) can specifically bind to wild-type human CD47, and does not exhibit comparable binding (e.g., does not exhibit detectable binding) to other proteins present in a sample.

In some embodiments, an anti-CD47 binding agent (e.g., anti-CD47 antibody) “specifically binds” wild-type CD47 polypeptide if it binds to or associates with the wild-type CD47 polypeptide with an affinity or Kₐ (that is, an equilibrium association constant of a particular binding interaction with units of 1/M) of, for example, greater than or equal to about 105 M⁻¹. In certain embodiments, the antibody binds to the wild-type CD47 polypeptide with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding refers to binding with a Kₐ of at least 10⁷ M⁻¹, at least 10⁸ M⁻¹, at least 109 M⁻¹, at least 10¹⁰ M⁻¹, at least 10¹¹ M¹, at least 10¹² M¹, at least 10¹³ M¹, or greater. Alternatively, affinity may be defined as an equilibrium dissociation constant (K_{D}) of a particular binding interaction with units of M (e.g., 10⁻⁵ M to 10⁻¹ M, or less). In some embodiments, specific binding means the polypeptide binds to the wild-type CD47 polypeptide with a K_{D} of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10¹² M or less. The binding affinity of the polypeptide for the wild-type CD47 polypeptide can be readily determined using conventional techniques, e.g., by competitive ELISA (enzyme-linked immunosorbent assay), equilibrium dialysis, by using surface plasmon resonance (SPR) technology (e.g., the BIAcore 2000 instrument, using general procedures outlined by the manufacturer); by radioimmunoassay; or the like.

The nucleic acids and proteins of the present disclosure may be recombinant nucleic acids or proteins. As used herein, with respect to a protein, the term “recombinant” means having an altered amino acid sequence as a result of the application of genetic engineering techniques to nucleic acids that encode the protein, and cells or organisms that express the protein. With respect to a nucleic acid, the term “recombinant” means having an altered nucleic acid sequence as a result of the application of genetic engineering techniques. Genetic engineering techniques include, but are not limited to, PCR and DNA cloning technologies; transfection, transformation and other gene transfer technologies; homologous recombination; site-directed mutagenesis; and gene fusion. In accordance with this definition, a protein having an amino acid sequence identical to a naturally occurring protein, but produced by cloning and expression in a heterologous host, is not considered recombinant.

The nucleotide sequences of the nucleic acids of the present may be codon-optimized. “Codon-optimized” refers to changes in the codons of the polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, a nucleic acid of the present disclosure encoding the CD47 polypeptide may be codon-optimized for optimal production from the host organism selected for expression, e.g., human cells, such as human immune cells (e.g., human T cells).

Also provided by the present disclosure are expression constructs comprising any of the nucleic acids of the present disclosure. As used herein, an “expression construct” is a circular or linear polynucleotide (a polymer composed of naturally-occurring and/or non-naturally-occurring nucleotides) comprising a region that encodes a CD47 polypeptide of the present disclosure operably linked to a suitable promoter, e.g., a constitutive or inducible promoter.

The expression constructs (e.g., vectors) can be suitable for replication and integration in prokaryotes, eukaryotes, or both. The expression constructs may contain functionally appropriately oriented transcription and translation terminators, initiation sequences, and promoters useful for regulation of the expression of the CD47 polypeptide. The expression constructs optionally contain generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

To obtain high levels of expression of a cloned nucleic acid it is common to construct expression constructs which typically contain a strong promoter to direct transcription, a ribosome binding site for translational initiation, and a transcription/translation terminator, each in functional orientation to each other and to the protein-encoding sequence. Examples of regulatory regions suitable for this purpose in E. coli are the promoter and operator region of the E. coli tryptophan biosynthetic pathway, the leftward promoter of phage lambda (P_{L}), and the L-arabinose (araBAD) operon. The inclusion of selection markers in DNA vectors transformed in E. coli is also useful. Examples of such markers include genes specifying resistance to ampicillin, tetracycline, or chloramphenicol. Expression systems for expressing the selection system components are available using, for example, E. coli, Bacillus sp. and Salmonella. E. coli systems may also be used.

### Cells and Compositions

Aspects of the present disclosure further include cells. In certain embodiments, such cells include any of the nucleic acids of the present disclosure encoding any of the CD47 polypeptides described herein.

In some instances, a cell of the present disclosure comprises an expression construct of the present disclosure, where the cell expresses the CD47 polypeptide on its surface. According to some embodiments, a cell of the present disclosure comprises any of the nucleic acids of the present disclosure, where the nucleic acid is a transgene integrated into the genome of the cell or maintained episomally in the cell, and wherein the cell expresses the CD47 polypeptide on its surface. In certain embodiments, the transgene comprises the nucleic acid operably linked to one or more expression control sequences. In some instances, the transgene is operably linked to an endogenous promoter of the cell. According to some embodiments, the nucleic acid is the endogenous CD47 gene of the cell which has been mutated to encode the CD47 polypeptide comprising the mutant CD47 Ig-like domain, and wherein the cell expresses the CD47 polypeptide on its surface.

Various convenient methods of introducing the nucleic acid (e.g., transgene, expression construct, or the like) into the cell, or mutating the genome of the cell, for genetic modification of the cell may be employed including but not limited to, e.g., transfection of reagents and/or nucleic acids encoding such agents, transduction of genetic modification reagents, nucleofection and/or electroporation of genetic modification reagents, and the like. In some instances, a vector, e.g., a viral vector or a non-viral vector may be employed. In some instances, the components of the vector may include nucleic acids, proteins, or a combination thereof. Any convenient viral or non-viral vector may be employed including but not limited to e.g., lipid nanoparticle (LNP) vectors.

Vectors may be configured to contain all, or less than all, of the components necessary for performing a desired genetic modification. For example, in some instances, a vector may include all components sufficient for performing a genetic modification at a targeted locus. In some instances, a vector may include less than all of the components needed for performing a genetic modification and the remaining components may be delivered by other means, e.g., another different vector, transduction, transfection, or the like. In some instances, components, e.g., nucleic acid and protein components, of a targeting system may be pre-complexed prior to delivery, including where such components are pre-complexed within a delivery vector. For example, in some instances nucleic acid (e.g., a gRNA, etc.) and protein (e.g., nuclease(s) or base editing protein(s), etc.) editing reagents of an editing system may be complexed as ribonucleoprotein (RNP) for delivery to a cell population for genetic modification.

Any convenient and appropriate genetic modification system may be employed to introduce one or more of the genetic modifications described herein. Methods of site-directed introduction of a desired genetic modification will vary and may include introducing one or more site directed cleavage events, e.g., through the use of one or more site-directed nucleases (e.g., a CRISPR/Cas9 nuclease, a TALEN nuclease, a ZFN, and the like). Site-directed cleavage may include double and/or single strand breaks where applicable. In some instances, site-directed cleavage is followed by a specific repair event at the site cleaved by the site-directed nuclease, e.g., to introduce a desired edit, such as e.g., a substitution, insertion, deletion, or the like. Such methods of specific repair may include, e.g., homologous recombination, including homology directed repair (HDR), e.g., in the presence of a nucleic acid that includes homology regions to guide the repair. In some instances, site-directed cleavage may be employed to introduce a gene disruption and/or knock-out, e.g., without employing a specific repair event, e.g., through cellular processes following site-directed cleavage such as e.g., non-homologous end joining (NHEJ). In some instances, site-directed introduction of a desired genetic modification may employ a base editing system that does not introduce a double strand cleavage event, such as but not limited to e.g., CRISPR protein-guided based editing systems, such as e.g., dCas9-deaminase fusion protein systems including cytosine base editor (CBE) and adenine base editor (ABE) systems. In some instances, useful base editing systems introduce a single base change, e.g., without cleavage of the phosphodiester nucleic acid backbone.

Various genetic modification compositions may be employed and such compositions will vary, e.g., based on the genetic modification system employed, the type of genetic modification desired, the sequence of a targeted locus or loci, etc. Useful genetic modification compositions may include e.g., CRISPR/Cas9 editing compositions, e.g., including a Cas9 protein, or a nucleic acid encoding a Cas9 protein, and gRNAs or a sgRNA or a nucleic acid encoding the gRNAs or sgRNA; TALEN editing compositions, including e.g., a TALEN nuclease or TALEN nuclease pair, or a nucleic acid encoding a TALEN nuclease or TALEN nuclease pair; ZFN editing compositions, including e.g., a ZFN nuclease or ZFN nuclease pair, or a nucleic acid encoding a ZFN nuclease or ZFN nuclease pair; base-editing editing compositions e.g., including a CRISPR-protein-guided-base-editing protein, or a nucleic acid encoding a CRISPR-protein-guided-base-editing protein, and gRNAs or a sgRNA or a nucleic acid encoding the gRNAs or sgRNA; and the like.

According to some embodiments, useful genetic modification (sometimes referred to herein as “editing compositions”) will include a CRISPR-Cas protein, such as e.g., a Cas9 protein, or a polynucleotide encoding a CRISPR-Cas protein and guide RNA (gRNA) or a polynucleotide encoding gRNA. As used herein, the term “gRNA” generally encompasses either two-component guide systems (e.g., two gRNAs) as well as single guide RNA (sgRNA) systems, unless inappropriate and/or denoted otherwise. In some instances, the gRNA or multiple gRNAs may be configured and employed to target a desired locus as described herein or one or more elements thereof such as one of more exons of a gene present at the locus. For example, in some instances, a gRNA or multiple gRNAs may be configured and employed to target a locus or one or more elements thereof, such as e.g., one or more exons of the locus.

In certain embodiments, the genetic modification may include the use of a Cas9 nuclease, including natural and engineered Cas9 nucleases, as well as nucleic acid sequences encoding the same. Useful Cas9 nucleases include but are not limited to e.g., Streptococcus pyogenes Cas9 and variants thereof, Staphylococcus aureus Cas9 and variants thereof, Actinomyces naeslundii Cas9 and variants thereof, Cas9 nucleases also include those discussed in PCT Publications Nos. WO 2013/176772 and WO2015/103153 and those reviewed in e.g., Makarova et al. (2011) Nature Reviews Microbiology 9:467-477, Makarova et al. (2011) Biology Direct 6:38, Haft et al. (2005) PLOS Computational Biology 1:e60 and Chylinski et al. (2013) RNA Biology 10:726-737, the disclosures of which are incorporated herein by reference in their entirety. In some instances, a non-Cas9 CRISPR nuclease (or engineered variant thereof) may be employed, including but not limited to e.g., Cpf1 or Cpf1 variant.

The CRISPR system offers significant versatility in gene editing in part because of the small size and high frequency of necessary sequence targeting elements within host genomes. CRISPR guided Cas9 nuclease requires the presence of a protospacer adjacent motif (PAM), the sequence of which depends on the bacteria species from which the Cas9 was derived (e.g. for Streptococcus pyogenes the PAM sequence is “NGG”) but such sequences are common throughout various target nucleic acids. The PAM sequence directly downstream of the target sequence is not part of the guide RNA but is obligatory for cutting the DNA strand. Synthetic Cas9 nucleases have been generated with novel PAM recognition, further increasing the versatility of targeting, and may be used in the methods described herein. Cas9 nickases (e.g., Cas9 (D10A) and the like) that cleave only one strand of target nucleic acid as well as endonuclease deficient (i.e., “dead”) dCas9 variants with additional enzymatic activities added by an attached fusion protein have also been developed.

In certain embodiments, a method of genetic modification may include the use of a zinc-finger nuclease (ZFN). ZFNs consist of the sequence-independent Fokl nuclease domain fused to zinc finger proteins (ZFPs). ZFPs can be altered to change their sequence specificity. Cleavage of targeted dsDNA involves binding of two ZFNs (designated left and right) to adjacent half-sites on opposite strands with correct orientation and spacing, thus forming a Fokl dimer. Dimerization increases ZFN specificity significantly. Three or four finger ZFPs target about 9 or 12 bases per ZFN, or about 18 or 24 bases for the ZFN pair. The specificity, efficiency and versatility of targeting and replacement of homologous recombination is greatly improved through the combined use of various homology-directed repair strategies and ZFNs (see e.g., Urnov et al. (2005) Nature. 435(7042):646-5; Beumer et al (2006) Genetics. 172(4):2391-2403; Meng et al (2008) Nat Biotechnol. 26(6):695-701; Perez et al. (2008) Nat Biotechnol. 26(7):808-816; Hockemeyer et al. (2009) Nat Biotechnol. 27(9):851-7; the disclosures of which are incorporated herein by reference in their entirety). In general, one ZFN site can be found every 125-500 bp of a random genomic sequence, depending on the assembly method. Methods for identifying appropriate ZFN targeting sites include computer-mediated methods e.g., as described in e.g., Cradick et al. (2011) BMC Bioinformatics. 12:152, the disclosure of which is incorporated herein by reference in its entirety.

According to some embodiments, a method of genetic modification may include the use of a transcription activator-like effector nuclease (TALEN). Similar in principle to the ZFN nucleases, TALENs utilize the sequence-independent Fokl nuclease domain fused to Transcription activator-like effectors (TALEs) proteins that, unlike ZNF, individually recognize single nucleotides. TALEs generally contain a characteristic central domain of DNA-binding tandem repeats, a nuclear localization signal, and a C-terminal transcriptional activation domain. A typical repeat is 33-35 amino acids in length and contains two hypervariable amino acid residues at positions 12 and 13, known as the “repeat variable di-residue” (RVD). An RVD is able to recognize one specific DNA base pair and sequential repeats match consecutive DNA sequences. Target DNA specificity is based on the simple code of the RVDs, which thus enables prediction of target DNA sequences. Native TALEs or engineered/modified TALEs may be used in TALENs, depending on the desired targeting. TALENs can be designed for almost any sequence stretch. Merely the presence of a thymine at each 5′ end of the DNA recognition site is required. The specificity, efficiency and versatility of targeting and replacement of homologous recombination is greatly improved through the combined use of various homology-directed repair strategies and TALENs (see e.g., Zu et al. (2013) Nature Methods. 10:329-331; Cui et al. (2015) Scientific Reports 5:10482; Liu et al. (2012) J. Genet. Genomics. 39:209-215, Bedell et al. (2012) Nature. 491:114-118, Wang et al. (2013) Nat. Biotechnol. 31:530-532; Ding et al. (2013) Cell Stem Cell. 12:238-251; Wefers et al. (2013) Proc. Natl. Acad. Sci. U.S.A, 110:3782-3787; the disclosures of which are incorporated herein by reference in their entirety).

In certain embodiments, a method of genetic modification may include the use of a base editor system, including but not limited to e.g., base editor systems employing a fusion protein comprising a programable DNA binding protein, a nucleobase editor and gRNA, and the like. Base editing will generally not rely on HDR and/or NHEJ and will generally not result in or require the cleavage of phosphodiester bonds on both backbones of dsDNA. Thus, base editing may, in some instances, employ RNA-guided (i.e., “programable”) DNA binding proteins, such as Cas nucleases, that do not cause double-strand breaks, such as e.g., nuclease-deficient or nuclease-defective Cas proteins, such as e.g., a dCas9 or a Cas9 nickase. Useful examples of base editors and base editing systems, including base editor encoding nucleic acids, include but are not limited to BE1, BE2, BE3 (Komor et al., 2016); Target-AID (Nishida et al., 2016); SaBE3, BE3 PAM variants, BE3 editing window variants (Kim et al., 2017); HF-BE3 (Rees et al., 2017); BE4 and BE4-Gam; AID, CDA1 and APOBEC3G BE3 variants (Komor et al., 2017); BE4max, ArcBe4max, ABEmax (Koblan et al., 2018); Adenine base editors (ABE7.10) (Gaudelli et al., 2017); ABE8 (Richter et al., 2020); ABE8e (Gaudelli et al., 2020); A&C-BEmax (Zhang et al., 2020); SPACE (Grunewald et al., 2020); and the like; the preceding references being incorporated by reference herein in their entirety.

Other useful components, e.g., of transgenes, of expression cassettes, of editing compositions, of vectors, or the like, may include promoter sequences (e.g., constitutive, tissue-specific, etc.), signal peptide sequences, poly(A) sequences, terminators, translational regulatory sequences such as ribosome binding sites and internal ribosome entry sites, enhancers, silencers, insulators, boundary elements, replication origins, matrix attachment sites and/or locus control regions. Furthermore, multiple gene products can be expressed from one nucleic acid, for example by linking individual components (transgenes) in one open reading frame separated, for example, by a self-cleaving 2A peptide or IRES sequence.

Examples of useful promoters include, for example, viral simian virus 40 (SV40) (e.g., early or late), cytomegalovirus (CMV) (e.g., immediate early), Moloney murine leukemia virus (MoMLV), MND (myeloproliferative sarcoma virus enhancer, negative control region deleted, dl587rev primer-binding site substituted), Rous sarcoma virus (RSV), herpes simplex virus (HSV), spleen focus-forming virus (SFFV) promoters and the like. In certain embodiments, the promoter may be inducible, such that transcription of all or part of the viral genome will occur only when one or more induction factors are present. Induction factors include, but are not limited to, one or more chemical compounds or physiological conditions, e.g., temperature or pH, in which the host cells are cultured. In some instances, the promoter may be constitutive. In some instances, the promoter may cause preferential expression in a desired cell-type or tissue, e.g., the promoter may be cell-type or tissue specific.

Vectors, including retroviral vectors, e.g., lentivirus vectors, may include (or exclude as desired where appropriate) various elements, including cis-acting elements, such as promoters, long terminal repeats (LTR), and/or elements thereof, including 5′ LTRs and 3′ LTRs and elements thereof, central polypurine tract (cPPT) elements, DNA flap (FLAP) elements, export elements (e.g., rev response element (RRE), hepatitis B virus post-transcriptional regulatory element (HPRE), etc.), posttranscriptional regulatory elements (e.g., woodchuck hepatitis virus posttranscriptional regulatory element (WPRE), hepatitis B virus regulatory element (HPRE), etc.), polyadenylation sites, transcription termination signals, insulators elements (e.g., β-globin insulator, e.g., chicken HS4), and the like.

Functional integration of a transgene may be achieved through various means, including through the use of integrating vectors, including viral and non-viral vectors. In some instances, a retroviral vector, e.g., a lentiviral vector, may be employed. In some instances, a non-retroviral integrating vector may be employed. An integrating vector may be contacted with the targeted cells in a suitable transduction medium, at a suitable concentration (or multiplicity of infection), and for a suitable time for the vector to infect the target cells, facilitating functional integration of the transgene. By “functionally integrated”, as used herein, is generally meant that the transgene is integrated into the genome of the cell in such a way that the encoded gene product is expressed. Expression of the encoded gene product may be controlled, in whole or in part, by endogenous components of the cell or exogenous (including heterologous) components included in the transgene. For example, in some instances, expression of the encoded gene product may be controlled by one or more endogenous regulatory elements, e.g., promoter, enhancer, etc., at or near the genomic locus into which the transgene is inserted. In some instances, expression of the encoded gene product may be controlled by one or more exogenous (including heterologous) regulatory elements, e.g., promoter, enhancer, etc., present in the transgene, and operably linked to the encoded gene product, prior to insertion.

In certain embodiments, the genetically modifying comprises inactivating one or more endogenous genes of the cell. For example, the genetically modifying may comprise inactivating (e.g., knocking out) one or more of the endogenous genes (e.g., the endogenous wild-type CD47 gene), and/or transiently or permanently downregulating expression of a gene (e.g., knockdown of the endogenous wild-type CD47 gene), e.g., via RNA interference, morpholino, and/or the like.

Suitable approaches for making gene knockouts, knock-ins, and downregulating genes are well known in the art.

According to some embodiments, a cell of the present disclosure is a therapeutic cell. As used herein, a therapeutic cell is a cell for use in a cell therapy (e.g., adoptive cell therapy). Cell therapy refers to the transfer of autologous or allogeneic cellular material into a patient for medical purposes.

In certain embodiments, when the cell is a therapeutic cell, the therapeutic cell is a therapeutic immune cell. Non-limiting examples of therapeutic immune cells of the present disclosure include a therapeutic T cell, a therapeutic natural killer T (NKT) cell, a therapeutic natural killer (NK), and a therapeutic macrophage. In some embodiments, the therapeutic immune cell is a tumor infiltrating lymphocyte (TIL).

In some instances, the therapeutic immune cell is a therapeutic T cell. Non-limiting examples of T cells include naive T cells (T_{N}), cytotoxic T cells (T_{CTL}), memory T cells (T_{MEM}), T memory stem cells (T_{SCM}), central memory T cells (T_{CM}), effector memory T cells (T_{EM}), tissue resident memory T cells (T_{RM}), effector T cells (T_{EFF}), regulatory T cells (T_{REGS}), helper T cells (T_{H}, T_{H}1, T_{H}2, T_{H}17), CD4+ T cells, CD8+ T cells, virus-specific T cells, alpha beta T cells (T_{αβ}), and gamma delta T cells (T_{γδ}).

According to some embodiments, a therapeutic immune cell of the present disclosure comprises a nucleic acid that encodes an engineered receptor, where the therapeutic immune cell further expresses the engineered receptor on its surface. In some instances, the engineered receptor is a chimeric antigen receptor (CAR), a T cell receptor (TCR) such as a recombinant TCR, a chimeric cytokine receptor (CCR), a synthetic notch receptor (synNotch), a Modular Extracellular Sensor Architecture (MESA) receptor, a Tango receptor, a ChaCha receptor, a generalized extracellular molecule sensor (GEMS) receptor, a growth factor receptor, a cytokine receptor, a chemokine receptor, a switch receptor, an adhesion molecule, an integrin, an inhibitory receptor, a stimulatory receptor, an immunoreceptor tyrosine-based activation motif (ITAM)-containing receptor, or an immunoreceptor tyrosine-based inhibition motif (ITIM)-containing receptor. In certain embodiments, the engineered receptor is a CAR. In some instances, the engineered receptor is a TCR.

In certain embodiments, the engineered receptor comprises an extracellular binding domain that binds a tumor antigen expressed on the surface of a cancer cell. Non-limiting examples of such tumor antigens include 5T4, AXL receptor tyrosine kinase (AXL), B7-H3, B-cell maturation antigen (BCMA), c-MET, C4.4a, carbonic anhydrase 6 (CA6), carbonic anhydrase 9 (CA9), Cadherin-6, CD19, CD20, CD22, CD25, CD27L, CD30, CD33, CD37, CD44, CD44v6, CD56, CD70, CD74, CD79b, CD123, CD138, carcinoembryonic antigen (CEA), cKit, Cripto protein, CS1, delta-like canonical Notch ligand 3 (DLL3), endothelin receptor type B (EDNRB), ephrin A4 (EFNA4), epidermal growth factor receptor (EGFR), EGFRvIII, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), GD2 ganglioside (“GD2”), glycoprotein non-metastatic B (GPNMB), guanylate cyclase 2 C (GUCY2C), human epidermal growth factor receptor 2 (HER2), human epidermal growth factor receptor 3 (HER3), Integrin alpha, lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), programmed cell death receptor ligand 1 (PD-L1), programmed cell death receptor ligand 2 (PD-L2), prostate-specific membrane antigen (PSMA), protein tyrosine kinase 7 (PTK7), solute carrier family 44 member 4 (SLC44A4), SLIT like family member 6 (SLITRK6), STEAP family member 1 (STEAPI), tissue factor (TF), T cell immunoglobulin and mucin protein-1 (TIM-1), Tn antigen, trophoblast cell-surface antigen (TROP-2), Wilms' tumor 1 (WT1), and VEGF-A.

In certain embodiments, the engineered receptor (e.g., a CAR) comprises an extracellular binding domain that binds to CD47.

As described above, according to some embodiments, the engineered receptor is a CAR. The extracellular binding domain of the CAR may comprise a single chain antibody. The single-chain antibody may be a monoclonal single-chain antibody, a chimeric single-chain antibody, a humanized single-chain antibody, a fully human single-chain antibody, and/or the like. In one non-limiting example, the single chain antibody is a single chain variable fragment (scFv). In some embodiments, the extracellular binding domain of the CAR is a single-chain version (e.g., an scFv version) of an antibody approved by the United States Food and Drug Administration and/or the European Medicines Agency (EMA) for use as a therapeutic antibody. Non-limiting examples of single-chain antibodies which may be employed when the protein of interest is a CAR include single-chain versions (e.g., scFv versions) of Adecatumumab, Ascrinvacumab, Cixutumumab, Conatumumab, Daratumumab, Drozitumab, Duligotumab, Durvalumab, Dusigitumab, Enfortumab, Enoticumab, Figitumumab, Ganitumab, Glembatumumab, Intetumumab, Ipilimumab, Iratumumab, Icrucumab, Lexatumumab, Lucatumumab, Mapatumumab, Narnatumab, Necitumumab, Nesvacumab, Ofatumumab, Olaratumab, Panitumumab, Patritumab, Pritumumab, Radretumab, Ramucirumab, Rilotumumab, Robatumumab, Seribantumab, Tarextumab, Teprotumumab, Tovetumab, Vantictumab, Vesencumab, Votumumab, Zalutumumab, Flanvotumab, Altumomab, Anatumomab, Arcitumomab, Bectumomab, Blinatumomab, Detumomab, Ibritumomab, Minretumomab, Mitumomab, Moxetumomab, Naptumomab, Nofetumomab, Pemtumomab, Pintumomab, Racotumomab, Satumomab, Solitomab, Taplitumomab, Tenatumomab, Tositumomab, Tremelimumab, Abagovomab, Igovomab, Oregovomab, Capromab, Edrecolomab, Nacolomab, Amatuximab, Bavituximab, Brentuximab, Cetuximab, Derlotuximab, Dinutuximab, Ensituximab, Futuximab, Girentuximab, Indatuximab, Isatuximab, Margetuximab, Rituximab, Siltuximab, Ublituximab, Ecromeximab, Abituzumab, Alemtuzumab, Bevacizumab, Bivatuzumab, Brontictuzumab, Cantuzumab, Cantuzumab, Citatuzumab, Clivatuzumab, Dacetuzumab, Demcizumab, Dalotuzumab, Denintuzumab, Elotuzumab, Emactuzumab, Emibetuzumab, Enoblituzumab, Etaracizumab, Farletuzumab, Ficlatuzumab, Gemtuzumab, Imgatuzumab, Inotuzumab, Labetuzumab, Lifastuzumab, Lintuzumab, Lorvotuzumab, Lumretuzumab, Matuzumab, Milatuzumab, Nimotuzumab, Obinutuzumab, Ocaratuzumab, Otlertuzumab, Onartuzumab, Oportuzumab, Parsatuzumab, Pertuzumab, Pinatuzumab, Polatuzumab, Sibrotuzumab, Simtuzumab, Tacatuzumab, Tigatuzumab, Trastuzumab, Tucotuzumab, Vandortuzumab, Vanucizumab, Veltuzumab, Vorsetuzumab, Sofituzumab, Catumaxomab, Ertumaxomab, Depatuxizumab, Ontuxizumab, Blontuvetmab, Tamtuvetmab, or an antigen-binding variant thereof.

When the cells are engineered to express a recombinant receptor on the surface thereof, the receptor may include one or more linker sequences between the various domains. A “variable region linking sequence” is an amino acid sequence that connects a heavy chain variable region to a light chain variable region and provides a spacer function compatible with interaction of the two sub-binding domains so that the resulting polypeptide retains a specific binding affinity to the same target molecule as an antibody that includes the same light and heavy chain variable regions. A non-limiting example of a variable region linking sequence is a glycine-serine linker, such as a (G₄S)₃ linker as described above. In certain embodiments, a linker separates one or more heavy or light chain variable domains, hinge domains, transmembrane domains, co-stimulatory domains, and/or primary signaling domains. In particular embodiments, the receptor (e.g., CAR) includes one, two, three, four, or five or more linkers. In particular embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids in length.

In some embodiments, when the cells are engineered to express a recombinant receptor on the surface thereof, the antigen binding domain of the receptor (e.g., CAR) is followed by one or more spacer domains that moves the antigen binding domain away from the cell surface (e.g., the surface of a T cell (e.g., a CD8+ or CD4+ T cell) expressing the receptor) to enable proper cell/cell contact, antigen binding and/or activation. The spacer domain (and any other spacer domains, linkers, and/or the like described herein) may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain may include the amino acid sequence of a naturally occurring immunoglobulin hinge region or an altered immunoglobulin hinge region. In some embodiments, the spacer domain includes the CH2 and/or CH3 of IgG1, IgG4, or IgD. Illustrative spacer domains suitable for use in the receptors (e.g., CARs) described herein include the hinge region derived from the extracellular regions of type 1 membrane proteins such as CD8α and CD4, which may be wild-type hinge regions from these molecules or variants thereof. In certain embodiments, the hinge domain includes a CD8α hinge region. According to some embodiments, the hinge is a PD-1 hinge or CD152 hinge. In certain embodiments, the hinge is an IgG4 hinge.

The “transmembrane domain” (Tm domain) is the portion of the receptor (e.g., CAR) that fuses the extracellular binding portion and intracellular signaling domain and anchors the receptor to the plasma membrane of the cell (e.g., T-cell, such as a Treg). The Tm domain may be derived either from a natural, synthetic, semi-synthetic, or recombinant source. In some embodiments, the Tm domain is derived from (e.g., includes at least the transmembrane region(s) or afunctional portion thereof) of the alpha or beta chain of the T-cell receptor, CD35, CD3ζ, CD3γ, CD3δ, CD4, CD5, CD8α, CD9, CD16, CD22, CD27, CD28, CD33, CD37, CD45, CD64, CD80, CD86, CD134, CD137, CD152, CD154, or PD-1.

In one embodiment, a receptor (e.g., CAR) includes a Tm domain derived from CD28. In certain embodiments, a receptor includes a Tm domain derived from CD28 and a short oligo- or polypeptide linker, e.g., between 1, 2, 3, 4, 5, 6, 7, 8, 9, or 10 amino acids in length, that links the Tm domain and the intracellular signaling domain of the receptor. A glycine-serine linker may be employed as such a linker, for example.

The “intracellular signaling” domain of a receptor (e.g., a CAR) refers to the part of the receptor that participates in transducing the signal from binding to a target molecule/antigen into the interior of the cell to elicit cell function. Accordingly, the term “intracellular signaling domain” refers to the portion of a protein which transduces the signal and that directs the cell to perform a specialized function. To the extent that a truncated portion of an intracellular signaling domain is used, such truncated portion may be used in place of a full-length intracellular signaling domain as long as it transduces the signal. The term intracellular signaling domain is meant to include any truncated portion of an intracellular signaling domain sufficient for transducing signal.

Signals generated through the T cell receptor (TCR) alone are insufficient for full activation of the T cell, and a secondary or costimulatory signal is also required. Thus, T cell activation is mediated by two distinct classes of intracellular signaling domains: primary signaling domains that initiate antigen-dependent primary activation through the TCR (e.g., a TCR/CD3 complex) and costimulatory signaling domains that act in an antigen-independent manner to provide a secondary or costimulatory signal. As such, a receptor (e.g., CAR) expressed by a genetically modified cell may include an intracellular signaling domain that includes one or more (e.g., 1, 2, or more) “costimulatory signaling domains” and a “primary signaling domain.”

Primary signaling domains regulate primary activation of the TCR complex either in a stimulatory manner, or in an inhibitory manner. Primary signaling domains that act in a stimulatory manner may contain signaling motifs which are known as immunoreceptor tyrosine-based activation motifs (or “ITAMs”). Non-limiting examples of ITAM-containing primary signaling domains suitable for use in a receptor of the present disclosure include those derived from FcRγ, FcRβ, CD3γ, CD3δ, CD3ε, CD3ζ, CD22, CD79α, CD79β, and CD66δ. In certain embodiments, a receptor includes a CD34 primary signaling domain and one or more costimulatory signaling domains. The intracellular primary signaling and costimulatory signaling domains are operably linked to the carboxyl terminus of the transmembrane domain.

In some embodiments, when the cells of the present disclosure are engineered to express a recombinant receptor on the surface thereof, the receptor (e.g., CAR) includes one or more costimulatory signaling domains to enhance the efficacy and expansion of immune effector cells (e.g., T cells) expressing the receptor. As used herein, the term “costimulatory signaling domain” or “costimulatory domain” refers to an intracellular signaling domain of a costimulatory molecule or an active fragment thereof. Example costimulatory molecules suitable for use in receptors contemplated in particular embodiments include TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, KD2C, SLP76, TRIM, and ZAP70. In some embodiments, the receptor (e.g., CAR) includes one or more costimulatory signaling domains selected from the group consisting of 4-1BB (CD137), CD28, and CD134, and a CD3ζ primary signaling domain.

A receptor (e.g., CAR) may include any variety of suitable domains including but not limited to a leader sequence; hinge, spacer and/or linker domain(s); transmembrane domain(s); costimulatory domain(s); signaling domain(s) (e.g., CD3ζ domain(s)); ribosomal skip element(s); restriction enzyme sequence(s); reporter protein domains; and/or the like.

In certain embodiments, the therapeutic immune cell comprises an expression construct that encodes the therapeutic anti-CD47 binding agent, wherein the therapeutic T cell expresses and secretes the therapeutic anti-CD47 binding agent. The cell may be engineered to express and secrete any of the anti-CD47 binding agents described hereinabove, e.g., therapeutic anti-CD47 antibodies, soluble SIRPα decoys, and the like. The therapeutic anti-CD47 binding agent may be one approved for anti-CD47 therapy by the United States Food and Drug Administration (FDA) and/or the European Medicines Agency (EMA). According to some embodiments, the therapeutic anti-CD47 binding agent is a therapeutic anti-CD47 antibody. According to some embodiments, the therapeutic anti-CD47 antibody expressed and secreted by the cell is a therapeutic anti-CD47 antibody approved for anti-CD47 therapy by the United States FDA and/or the EMA. Non-limiting examples of therapeutic anti-CD47 antibodies which may be expressed and secreted by the cell include those having the six complementarity determining regions (CDRs) of lemzoparlimab, antibody B6H12, magrolimab (5F9), AK117, AO-176, CC-90002, DSP107, HX009, 1B1188, 1B1322, IMC-002, IMM0306, PF-07257876, SHR-1603, SRF231, STI-6643, TG-1801, TJ011133, or ZL-1201.

Also provided by the present disclosure are compositions comprising a population of the therapeutic immune cells of the present disclosure, e.g., any of the therapeutic immune cells described elsewhere herein.

Compositions suitable for adoptive cell therapy may be manufactured by expanding the therapeutic immune cells of the present disclosure. By “expanding” is meant the cells are cultured under conditions in which the cells proliferate. Suitable conditions may vary depending upon, e.g., the type of cells being expanded. Such conditions may include culturing the cell in a suitable container (e.g., a cell culture plate or well thereof, a cassette, tube, bottle or bag suitable for use in an automated therapeutic cell manufacturing system, e.g., a closed automated therapeutic cell manufacturing system such as the CliniMACS Prodigy® system by Miltenyi Biotec, the Xuri® cell expansion system by Cytiva, the G-Rex® cell expansion system by Wilson Wolf, the Quantum® cell expansion system from Terumo, the Cocoon® system by Lonza, or the like), in suitable medium (e.g., cell culture medium, such as RPMI, DMEM, IMDM, MEM, DMEM/F-12, or the like) at a suitable temperature (e.g., 32° C.-42° C., such as 37° C.) and pH (e.g., pH 7.0-7.7, such as pH 7.4) in an environment having a suitable percentage of CO2, e.g., 3% to 10%, such as 5%.

Methods for activating and expanding cells for therapy (e.g., therapeutic T cells and the like) are known in the art and are described, e.g., in U.S. Pat. Nos. 6,905,874; 6,867,041; and 6,797,514; and PCT Publication No. WO 2012/079000, the contents of which are hereby incorporated by reference in their entirety. In the example of T cells, such methods may include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). One example is the Dynabeads® system, a CD3/CD28 activator/stimulator system for physiological activation of human T cells. In other embodiments, the T cells are activated and stimulated to proliferate with feeder cells and appropriate antibodies and cytokines using methods such as those described in U.S. Pat. Nos. 6,040,177 and 5,827,642 and PCT Publication No. WO 2012/129514, the contents of which are hereby incorporated by reference in their entirety.

In certain embodiments, the cells are expanded using an automated system designed for the manufacture of therapeutic cells. Non-limiting examples of such systems include the CliniMACS Prodigy® system by Miltenyi Biotec, the Xuri® cell expansion system by Cytiva, the G-Rex® cell expansion system by Wilson Wolf, the Quantum® cell expansion system from Terumo, the Cocoon® system by Lonza, etc. Detailed guidance and protocols for manufacturing therapeutic cells on such systems are available from the providers of such systems.

Harvested therapeutic immune cell populations may be present in any suitable container (e.g., a culture vessel, tube, flask, vial, cryovial, cryo-bag, etc.) and may be employed (e.g., administered to a subject) using any suitable delivery method and/or device. Such populations of cells and pharmaceutical compositions may be prepared and/or used fresh or may be cryopreserved. In some instances, populations of therapeutic cells and pharmaceutical compositions thereof may be prepared in a “ready-to-use” format, including e.g., where the therapeutic cells are present in a suitable diluent and/or at a desired delivery concentration (e.g., in unit dosage form) or a concentration that can be readily diluted to a desired delivery concentration (e.g., with a suitable diluent or media). Populations of therapeutic cells and pharmaceutical compositions thereof may be prepared in a delivery device or a device compatible with a desired delivery mechanism or the desired route of delivery, such as but not limited to e.g., a syringe, an infusion bag, or the like.

In some instances, the present disclosure provides one or a plurality of cell therapy doses, e.g., each contained in suitable container. Cell therapy doses may be generated through a variety of methods. Aliquoting expanded populations of therapeutic cells into cell therapy doses may be performed by a variety of means. In some instances, a cell therapy dose includes, e.g., at least 10 million, at least 25 million, at least 50 million, at least 75 million, at least 100 million, at least 250 million, at least 500 million, at least 750 million, at least 1 billion, at least 2 billion, at least 3 billion, at least 4 billion, at least 5 billion, at least 6 billion, at least 7 billion, at least 8 billion, at least 9 billion, at least 10 billion, at least 15 billion, at least 20 billion, at least 30 billion, at least 40 billion, at least 50 billion, at least 60 billion, at least 70 billion, at least 80 billion, at least 90 billion, or at least 100 billion therapeutic cells.

In certain embodiments, the compositions may include the therapeutic cells present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a nuclease inhibitor, glycerol, a chelating agent, and the like may be present in such compositions.

The compositions generally include a therapeutically effective amount of the cells. By “therapeutically effective amount” is meant a number of cells sufficient to produce a desired result, e.g., an amount sufficient to effect beneficial or desired therapeutic (including preventative) results, such as a reduction in a symptom of a disease or disorder associated, e.g., with the target cell or a population thereof, as compared to a control. An effective amount can be administered in one or more administrations.

A “therapeutically effective amount” of such cells may vary according to factors such as the disease state, age, sex, and weight of the subject, and the ability of the cells to elicit a desired response in the subject. A therapeutically effective amount is also one in which any toxic or detrimental effects of the cells are outweighed by the therapeutically beneficial effects. The term “therapeutically effective amount” includes an amount that is effective to “treat” a subject (e.g., a patient). When a therapeutic amount is indicated, the precise amount of the compositions contemplated in particular embodiments, to be administered, can be determined by a physician in view of the specification and with consideration of individual differences in age, weight, tumor size, extent of infection or metastasis, and condition of the patient (subject). In certain embodiments, a pharmaceutical composition of the present disclosure includes from 1×10⁶ to 5×10¹⁰ of the therapeutic immune cells of the present disclosure.

The cells of the present disclosure can be incorporated into a variety of formulations for therapeutic administration. More particularly, the cells of the present disclosure can be formulated for administration by combination with appropriate excipients, diluents and/or the like.

Formulations of the cells suitable for administration to a patient (e.g., suitable for human administration) are generally sterile and may further be free of detectable pyrogens or other contaminants contraindicated for administration to a patient according to a selected route of administration.

The cells may be formulated for parenteral (e.g., intravenous, intra-arterial, intraosseous, intramuscular, intracerebral, intracerebroventricular, intrathecal, subcutaneous, etc.) administration, or any other suitable route of administration.

An aqueous formulation of the cells may be prepared in a pH-buffered solution, e.g., at pH ranging from about 4.0 to about 7.0, or from about 5.0 to about 6.0, or alternatively about 5.5.

Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included in the formulation to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

In some embodiments, a composition includes cells of the present disclosure, and one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

### Methods

Aspects of the present disclosure further include methods of using the therapeutic immune cells of the present disclosure for therapy, e.g., to treat a cell proliferative disorder such as cancer. In certain embodiments, provided are methods of administering an adoptive cell therapy to a subject having cancer, where the subject is receiving an anti-CD47 therapy to treat the cancer. Such methods comprise administering to the subject a composition of the present disclosure (that is, a composition comprising any of the therapeutic immune cells of the present disclosure) in an amount effective to treat the cancer. In certain embodiments, a practitioner of the methods administers the anti-CD47 therapy to the subject in addition to the adoptive cell therapy.

According to some embodiments, the adoptive cell therapy is an adoptive T cell therapy, e.g., a CAR T cell therapy, a TIL therapy, a natural killer T (NKT) cell therapy, a therapy comprising administration of T cells expressing an engineered TCR, or the like. In some instances, the adoptive cell therapy is a natural killer (NK) cell therapy (e.g., a CAR NK cell therapy) or a macrophage therapy.

The therapeutic cells may be autologous/autogeneic (“self”) or non-autologous (“non-self,” e.g., allogeneic, syngeneic or xenogeneic). “Autologous” as used herein, refers to cells obtained from the subject to whom the therapeutic cells are later administered. “Allogeneic” as used herein refers to cells obtained from a donor other than the subject to whom the therapeutic cells are administered. In some embodiments, the cells (e.g., T cells) are cells obtained from a mammalian subject. In certain embodiments, the mammalian subject is a primate. In some embodiments, the cells are obtained from a human.

The subject to whom the adoptive cell therapy is administered is receiving an anti-CD47 therapy. As used herein, an “anti-CD47 therapy” is one where the subject receives one or more administrations of an anti-CD47 binding agent, where the CD47 polypeptide expressed on the surface of the therapeutic immune cells comprises a mutant CD47 Ig-like domain (e.g., a mutant BC loop) that reduces binding of the anti-CD47 binding agent to the CD47 polypeptide as compared to binding of the anti-CD47 binding agent to a wild-type CD47 polypeptide, and where the CD47 polypeptide retains binding to SIRPα.

Accordingly, the type of cells employed in the adoptive cell therapy (in terms of the particular CD47 polypeptide expressed by those cells) and the anti-CD47 binding agent are selected to be complimentary to each other such that the CD47 polypeptide expressed by the cells partially or completely escapes blockade by the anti-CD47 binding agent employed.

The therapeutic anti-CD47 binding agent of the anti-CD47 therapy may vary. Therapeutic anti-CD47 binding agents of interest include those that bind to wild-type CD47 and inhibit or block interaction between the bound CD47 and SIRPα. Therapeutic anti-CD47 binding agents of interest include antibodies, soluble SIRPα decoys, and the like. The therapeutic anti-CD47 binding agent may be one approved for anti-CD47 therapy by the United States Food and Drug Administration (FDA) and/or the European Medicines Agency (EMA). In certain embodiments, the therapeutic anti-CD47 binding agent is a therapeutic anti-CD47 antibody. According to some embodiments, the therapeutic anti-CD47 antibody is a therapeutic anti-CD47 antibody approved for anti-CD47 therapy by the United States FDA and/or the EMA. Non-limiting examples of therapeutic anti-CD47 antibodies for which the CD47 polypeptides may exhibit reduced binding include those having the six complementarity determining regions (CDRs) of lemzoparlimab, antibody B6H12, magrolimab (5F9), AK117, AO-176, CC-90002, DSP107, HX009, 1B1188, 1B1322, IMC-002, IMM0306, PF-07257876, SHR-1603, SRF231, STI-6643, TG-1801, TJ011133, or ZL-1201.

As summarized above, in some embodiments, the subject has cancer. The terms “cancer” and “cancerous” refer to or describe the physiological condition in mammals that is typically characterized by unregulated cell growth/proliferation. “Tumor”, as used herein, refers to all neoplastic cell growth and proliferation, whether malignant or benign, and all pre-cancerous and cancerous cells and tissues.

In some instances, the cancer comprises a solid tumor. According to some embodiments, the solid tumor is a carcinoma, lymphoma, blastoma, or sarcoma. When the solid tumor is a carcinoma, in certain embodiments, the carcinoma is a basal cell carcinoma, squamous cell carcinoma, renal cell carcinoma, ductal carcinoma in situ (DCIS), invasive ductal carcinoma, or adenocarcinoma. As will be appreciated upon review of the unexpected results in Experimental section herein, in embodiments where the cancer comprises a solid tumor, the method may produce a synergistic effect between the adoptive cell therapy and the anti-CD47 therapy.

According to some embodiments, the cancer comprises a hematological malignancy. For example, the subject treated by the methods of the present disclosure may have a hematological malignancy such as a leukemia, a lymphoma, or multiple myeloma.

According to some embodiments, the cancer of the subject is myelodysplastic syndrome (MDS), acute myeloid leukemia (AML), multiple myeloma (MM), Non-Hodgkin's lymphoma (NHL), non-small cell lung cancer, head and neck squamous cell carcinoma, gastroesophageal junction adenocarcinoma, gastric adenocarcinoma, diffuse large B cell lymphoma, follicular lymphoma, marginal zone lymphoma, mantle cell lymphoma, chronic lymphocytic lymphoma (CLL), B cell lymphoma, lung adenocarcinoma, osteosarcoma, ovarian cancer, or leiomyosarcoma.

Particular examples of cancers which the subject may have include renal cancer; kidney cancer; glioblastoma multiforme; metastatic breast cancer; breast carcinoma; breast sarcoma; neurofibroma; neurofibromatosis; pediatric tumors; neuroblastoma; malignant melanoma; carcinomas of the epidermis; leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone cancer and connective tissue sarcomas such as but not limited to bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangio sarcoma, neurilemmoma, rhabdomyosarcoma, and synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and ciliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; cervical carcinoma; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; colorectal cancer, KRAS mutated colorectal cancer; colon carcinoma; rectal cancers; liver cancers such as but not limited to hepatocellular carcinoma and hepatoblastoma, gallbladder cancers such as adenocarcinoma; cholangiocarcinomas such as but not limited to papillary, nodular, and diffuse; lung cancers such as KRAS-mutated non-small cell lung cancer, non-small cell lung cancer, squamous cell carcinoma (epidermoid carcinoma), adenocarcinoma, large-cell carcinoma and small-cell lung cancer; lung carcinoma; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, androgen-independent prostate cancer, androgendependent prostate cancer, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acrallentiginous melanoma; kidney cancers such as but not limited to renal cell cancer, adenocarcinoma, hypernephroma, fibrosarcoma, transitional cell cancer (renal pelvis and/or uterer); renal carcinoma; Wilms' tumor; and bladder cancers such as but not limited to transitional cell carcinoma, squamous cell cancer, adenocarcinoma, carcinosarcoma. In some embodiments, the cancer is myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, or papillary adenocarcinomas.

In some embodiments, administration of the adoptive cell therapy is specifically timed relative to administration of the anti-CD47 therapy. For example, in some embodiments, the adoptive cell therapy is administered so that a particular effect is observed (or expected to be observed, for example based on population studies showing a correlation between a given dosing regimen and the particular effect of interest).

In certain embodiments, desired relative dosing regimens for agents administered in combination may be assessed or determined empirically, for example using ex vivo, in vivo and/or in vitro models; in some embodiments, such assessment or empirical determination is made in vivo, in a patient population (e.g., so that a correlation is established), or alternatively in a particular subject of interest.

In some embodiments, the adoptive cell therapy and anti-CD47 therapy are administered according to an intermittent dosing regimen including at least two cycles. Where two or more agents are administered in combination, and each by such an intermittent, cycling, regimen, individual doses of different agents may be interdigitated with one another.

One exemplary protocol for interdigitating two intermittent, cycled dosing regimens, may include: (a) a first dosing period during which an effective amount the adoptive cell therapy is administered to a subject; (b) a first resting period; (c) a second dosing period during which an effective amount of the anti-CD47 therapy is administered to the subject; and (d) a second resting period.

In some embodiments, the first resting period and second resting period may correspond to an identical number of hours or days. Alternatively, in some embodiments, the first resting period and second resting period are different, with either the first resting period being longer than the second one or, vice versa. In some embodiments, each of the resting periods corresponds to 120 hours, 96 hours, 72 hours, 48 hours, 24 hours, 12 hours, 6 hours, 30 hours, 1 hour, or less. In some embodiments, if the second resting period is longer than the first resting period, it can be defined as a number of days or weeks rather than hours (for instance 1 day, 3 days, 5 days, 1 week, 2, weeks, 4 weeks or more).

If the first resting period's length is determined by existence or development of a particular biological or therapeutic event, then the second resting period's length may be determined on the basis of different factors, separately or in combination. Exemplary such factors may include type and/or stage of a cancer against which the agents are administered; identity and/or properties (e.g., pharmacokinetic properties) of the first agent, and/or one or more features of the patient's response to therapy with the first agent. In some embodiments, length of one or both resting periods may be adjusted in light of pharmacokinetic properties (e.g., as assessed via plasma concentration levels) of one or the other (or both) of the administered agents. For example, a relevant resting period might be deemed to be completed when plasma concentration of the relevant agent is below about 1 μg/ml, 0.1 μg/ml, 0.01 μg/ml or 0.001 μg/ml, optionally upon evaluation or other consideration of one or more features of the subject's response.

In certain aspects, the number of cycles for which a particular agent is administered may be determined empirically. Also, in some embodiments, the precise regimen followed (e.g., number of doses, spacing of doses (e.g., relative to each other or to another event such as administration of another therapy), amount of doses, etc.) may be different for one or more cycles as compared with one or more other cycles.

The adoptive cell therapy and anti-CD47 therapy may be administered via a route of administration independently selected from parenteral (e.g., by intravenous, intra-arterial, subcutaneous, intramuscular, or epidural injection), oral, topical, or nasal administration.

According to certain embodiments, the adoptive cell therapy and anti-CD47 therapy are both administered parenterally, either concurrently (in the same composition or separate compositions) or sequentially.

By treatment is meant at least an amelioration of one or more symptoms associated with the cancer of the subject, where amelioration is used in a broad sense to refer to at least a reduction in the magnitude of a parameter, e.g. symptom, associated with the cancer being treated. As such, treatment also includes situations where the cancer, or at least one or more symptoms associated therewith, are completely inhibited, e.g., prevented from happening, or stopped, e.g., terminated, such that the individual no longer suffers from the cancer, or at least the symptoms that characterize the cancer. With respect to cancer, in some embodiments, the treatment is effective to slow the growth of a tumor, reduce the size of a tumor, and/or the like.

The following examples are offered by way of illustration and not by way of limitation.

### EXPERIMENTAL

### Example 1—Pairing of CAR T and Anti-CD47 Therapies Leads to Loss of CAR T Anti-Tumor Efficacy In Vivo Due to T Cell Depletion

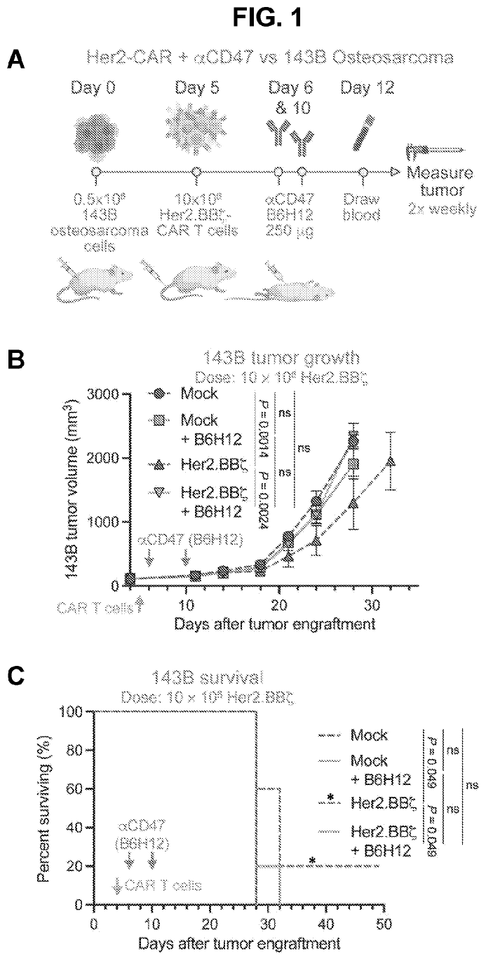

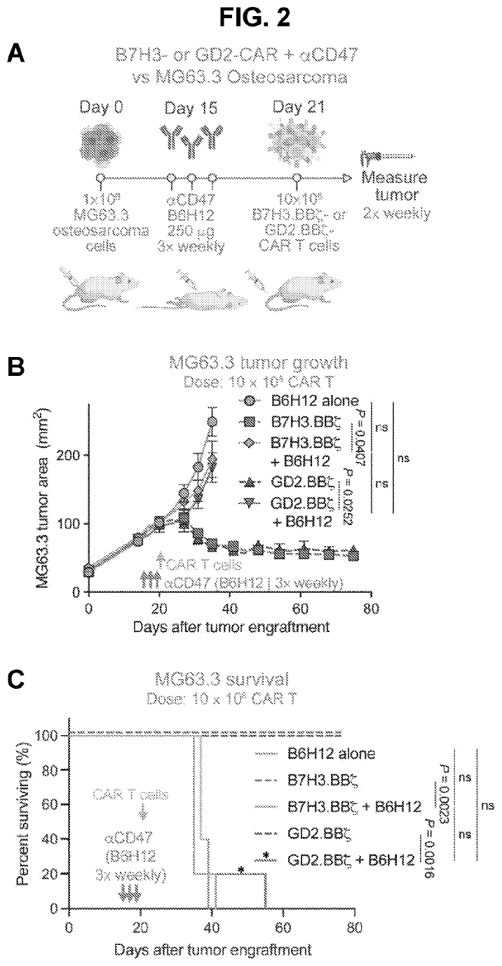

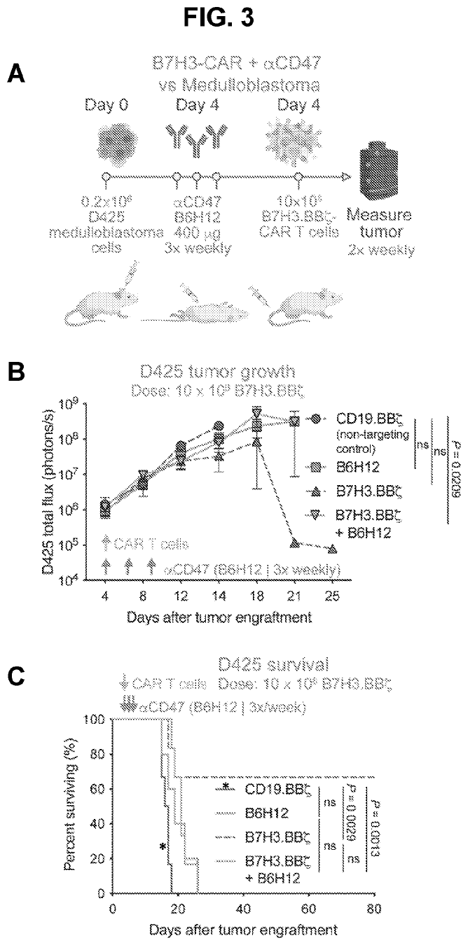

In this example, to test the hypothesis that augmenting macrophage phagocytosis via CD47 blockade could improve the efficacy of CAR based T cell therapy, the 143B osteosarcoma model was utilized, which had been previously shown to be a stringent model for CAR T therapy^{34,35}. Mice implanted orthotopically with 143B cells received Her2.BBζ-CAR T cells, followed by two doses of the αCD47 monoclonal antibody (mAb) B6H12³⁶⁻³⁹ (FIG. 1A). Antitumor effects with CAR T treatment alone were observed, but surprisingly, treatment with αCD47 ablated CAR T efficacy, demonstrating antagonistic activity (FIG. 1B-C). To interrogate this pairing in a less stringent model, the MG63.3 osteosarcoma, which had been shown previously to be sensitive to CAR T therapy^{34,40}, and sensitive to αCD47 plus αGD2 mAb therapy³⁹, was used. Mice implanted orthotopically with MG63.3 cells received B6H12, before administering either B7H3.BBζ- or GD2.BBζ-CAR T cells (FIG. 2A). The results confirmed antagonistic activity, with antitumor effects of each CAR T when administered as single agents, but no significant antitumor effect in animals receiving either CAR T therapy plus B6H12 (FIG. 2B-C). Similarly, B7H3.BBζ-CAR T mediated antitumor effects against D425 orthotopic medulloblastoma xenografts as previously reported⁴⁰, but no antitumor effects were observed when B7H3.BBζ-CAR T were co-administered with B6H112 (FIG. 3A-C).

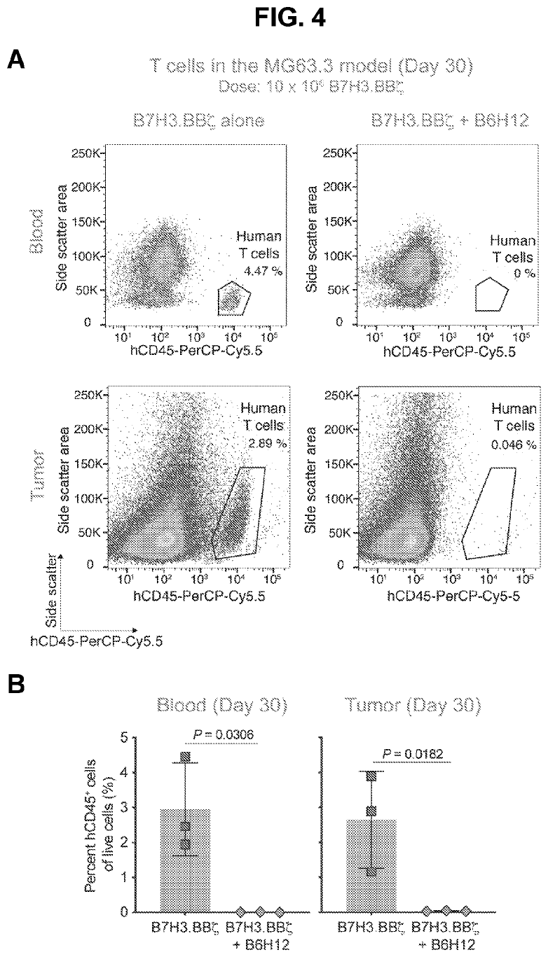

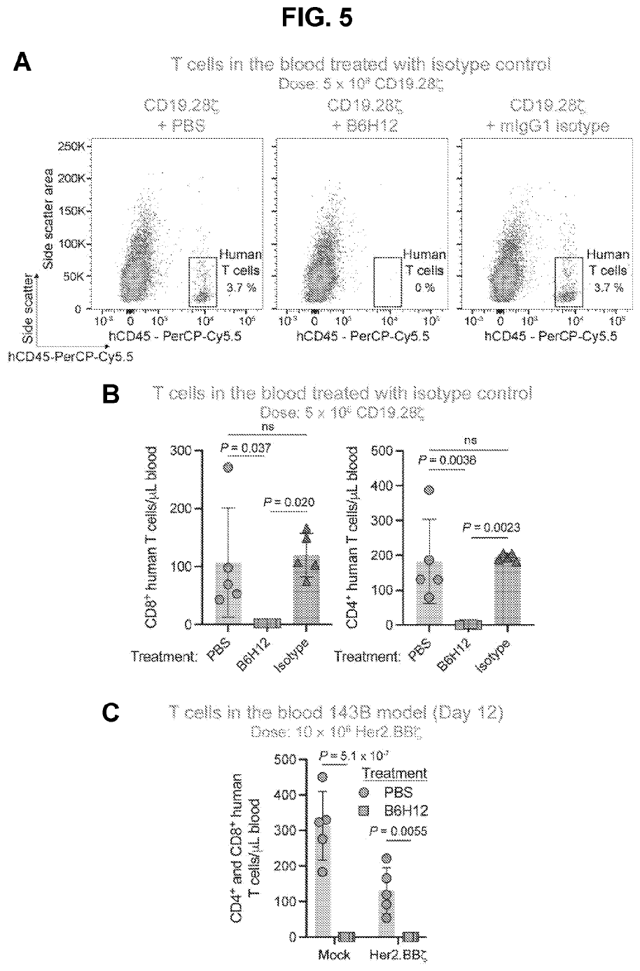

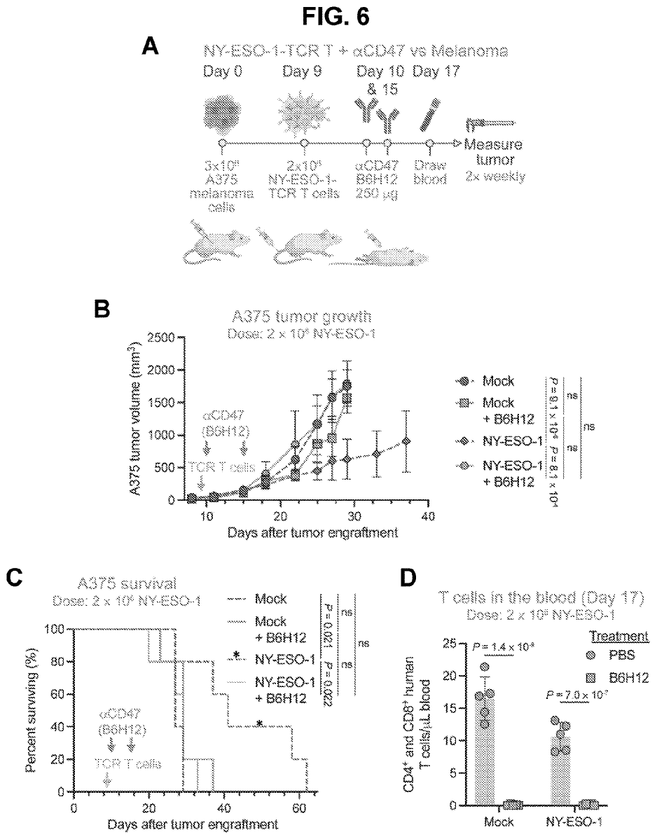

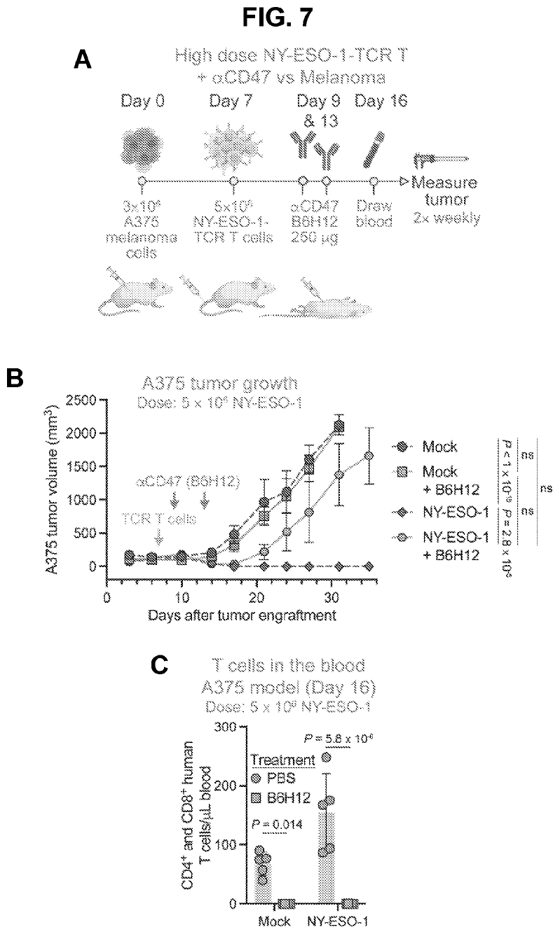

To interrogate the cause of therapeutic failure with dual treatment, human T cell levels were quantified in mice bearing MG63.3 tumors 15 days after B7H3.BBζ-CAR T±B6H12. Strikingly, human T cells were completely absent in tumor and blood in mice co-treated with B6H12 (FIG. 4A-B), while remaining detectable in CAR only and isotype control antibody treated animals (FIG. 5A-B). Depletion also occurred in the 143B model (FIG. 5C), with mock and CAR T cells completely absent from the blood of B6H12 co-treated mice. To explore whether B6H12 depleted adoptively transferred T cells that did not express CARs, αCD47 was paired with adoptive transfer of T cells engineered to express the widely validated NY-ESO-1 targeting TCR⁴¹. Mice were implanted with A375 melanoma cells, which display the NY-ESO-1 peptide, in a flank xenograft model and administered mock T cells, or NY-ESO-1-TCR T cells, ±B6H12 (FIG. 6A, FIG. 7A). As observed for CAR T cells, co-treatment of B6H12 and NY-ESO-1-TCR T cells at two different doses was associated with an absence of antitumor efficacy and depletion of adoptively transferred cells (FIG. 6B-D, FIG. 7B-C).

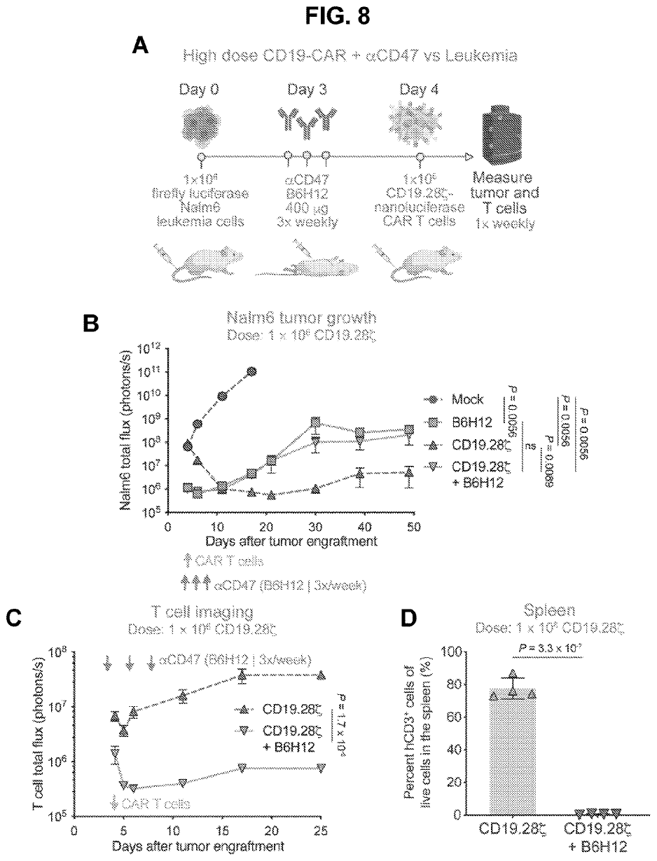

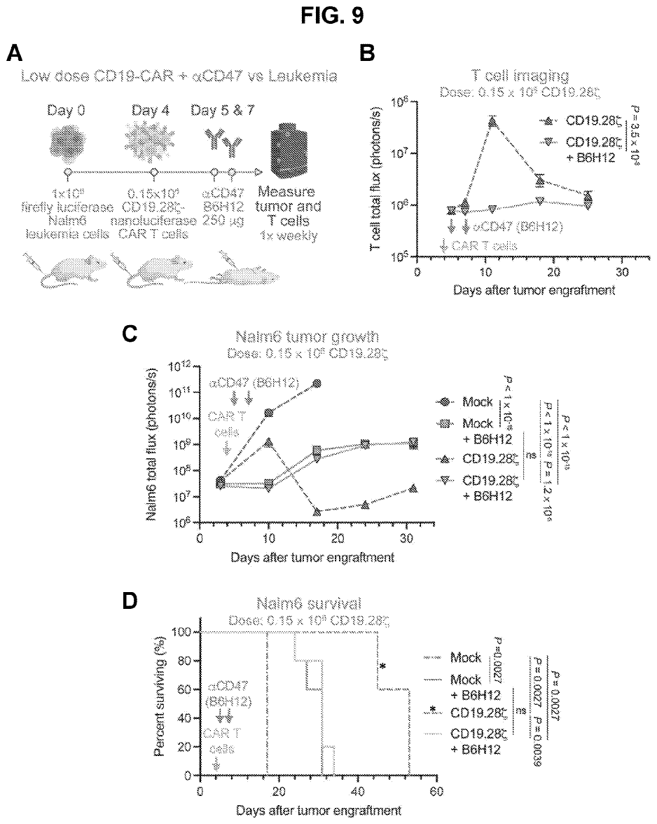

To characterize the kinetics of CAR T cell depletion in vivo in animals treated with adoptive T cell transfer plus B6H12, levels of CD19.28ζ-CAR T expressing nanoluciferase (CD19.28ζ-nLuc) were monitored via bioluminescent imaging (BLI) in animals inoculated with Nalm6 leukemia incorporating firefly luciferase (Nalm6-fLuc)±B6H12 (FIG. 8A, FIG. 9A)³⁴. Consistent with the results observed in solid tumor models, B6H12 completely ablated CD19.28ζ-CAR T efficacy (FIG. 8B, FIG. 9C-D) and BLI demonstrated a significant loss of CAR T signal shortly after B6H12 treatment (FIG. 8C, FIG. 9B). CAR T cells in the dual treatment group remained undetectable via BLI over the course of three weeks and were absent from spleens of mice at the conclusion of the experiment (FIG. 8D). Of note, the modest single agent efficacy of B6H12 in this system was not impacted by co-administration of CD19.28ζ-CAR T cells (FIG. 8B, FIG. 9C-D). Together, the data demonstrate that αCD47 induces rapid depletion of adoptively transferred non-genetically modified T cells, as well as T cells engineered to express a transgenic TCR or CARs with differing targeting (Her2, B7H3, GD2, and CD19) and costimulatory domains (4-1 BB and CD28).

### Example 2—CD47 Serves as a Critical “Don't Eat Me” Signal on CAR T Cells that Prevents Macrophage Mediated Phagocytosis and Overexpression of CD47 can Enhance CAR T Cell Persistence and Efficacy

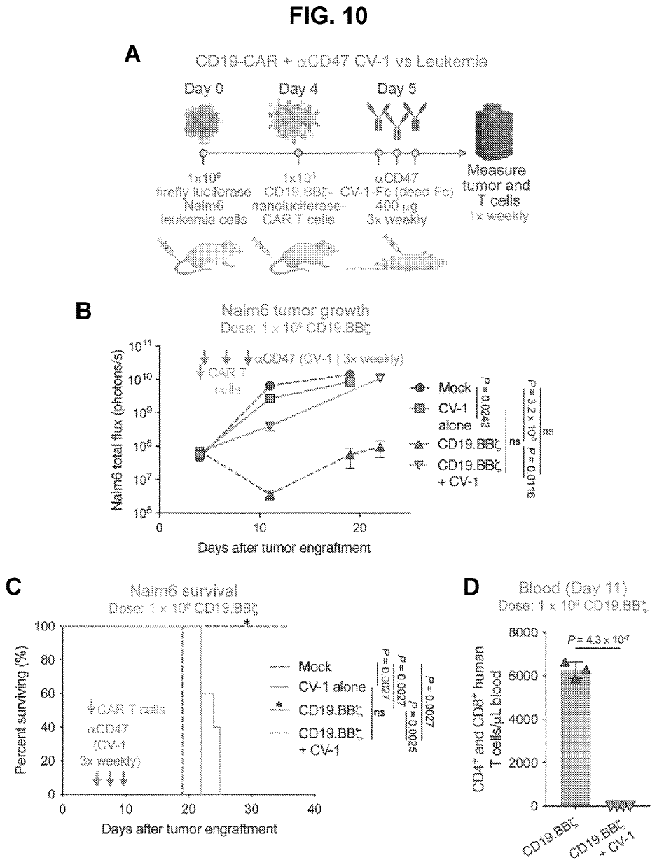

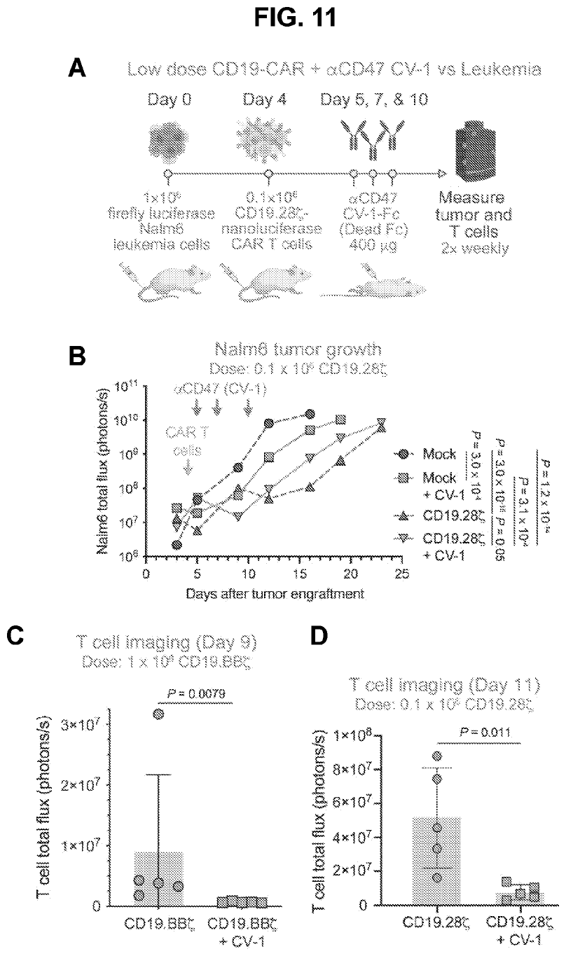

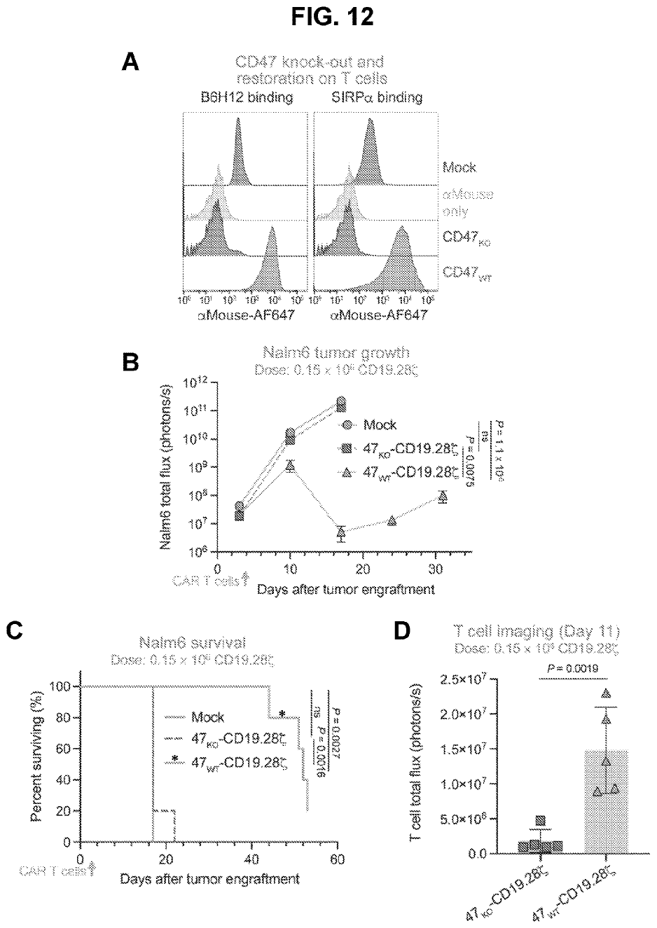

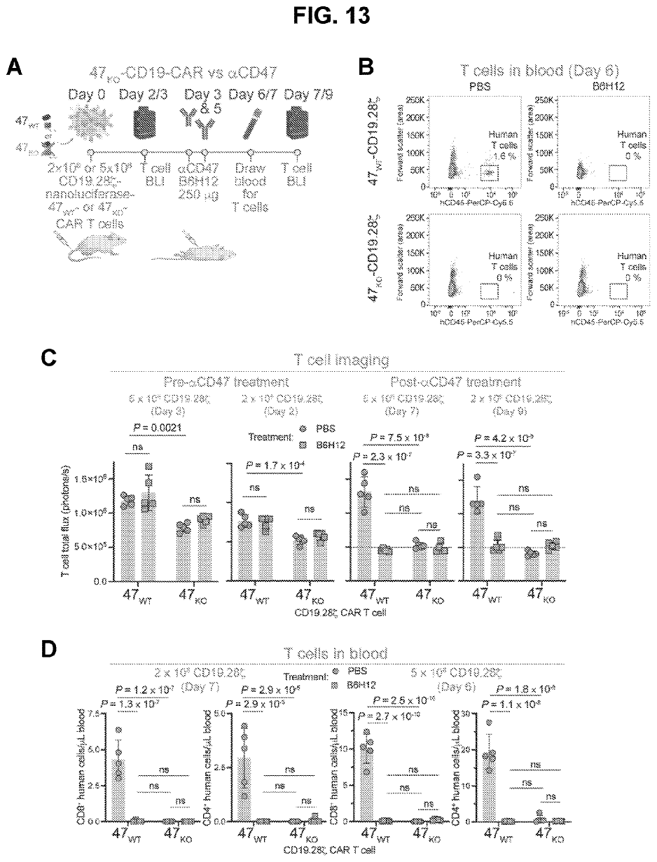

Next, it was determined whether T cell ablation in these models was due to antibody dependent phagocytosis that requires FcR engagement⁴²⁻⁴⁴ by administering CV-1⁴⁵, an engineered SIRPα Fc-fusion that binds CD47 with high affinity and blocks interaction with endogenous SIRPα, fused to an immunologically inert hIgG1 Fc-domain with LALA-PG mutations⁴⁴. Similar to results with B6H12, CV-1 co-treatment with either CD19.BBζ- or CD19.28ζ-CAR T led to loss of CAR T antitumor efficacy in Nalm6-fLuc bearing mice (FIG. 10A-C, FIG. 11A-B), and near total T cell depletion (FIG. 10D, FIG. 11C-D). Next, CD47 expression was tested as a requirement for survival of adoptively transferred T cells, by generating CD47 knock-out (47_{KO}) primary human T cells via CRISPR/Cas9, validated by loss of αCD47 and soluble SIRPα binding (FIG. 12A), which was then restored upon exogenous expression of full-length, wild-type CD47 in 47_{KO} cells (47_{WT}). Nalm6-fLuc bearing mice were treated with 47_{KO}- or 47_{WT}-CD19.28ζ-nLuc-CAR T and observed that 47_{WT}-CAR T cells expanded in vivo, mediated robust tumor control and significantly prolonged survival, whereas 47_{KO}-CAR T were depleted and delivered no anti-tumor activity or benefit in survival (FIG. 12B-D). Even in the absence of tumor, robust expansion of 47_{WT}-CAR T was observed, while 47_{KO}-CAR T were depleted to a similar degree as 47_{WT}-CAR T after B6H12 administration (FIG. 13A-D), demonstrating a role for macrophages in regulating homeostatic expansion of T cells.

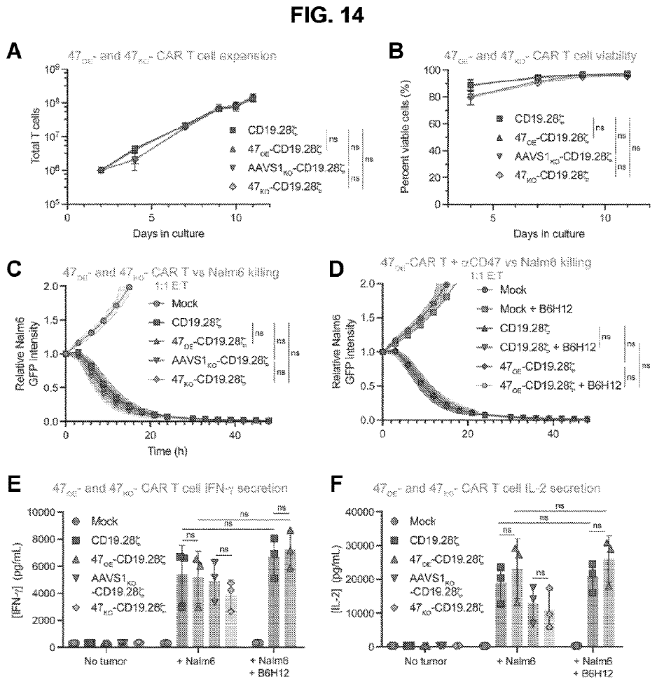

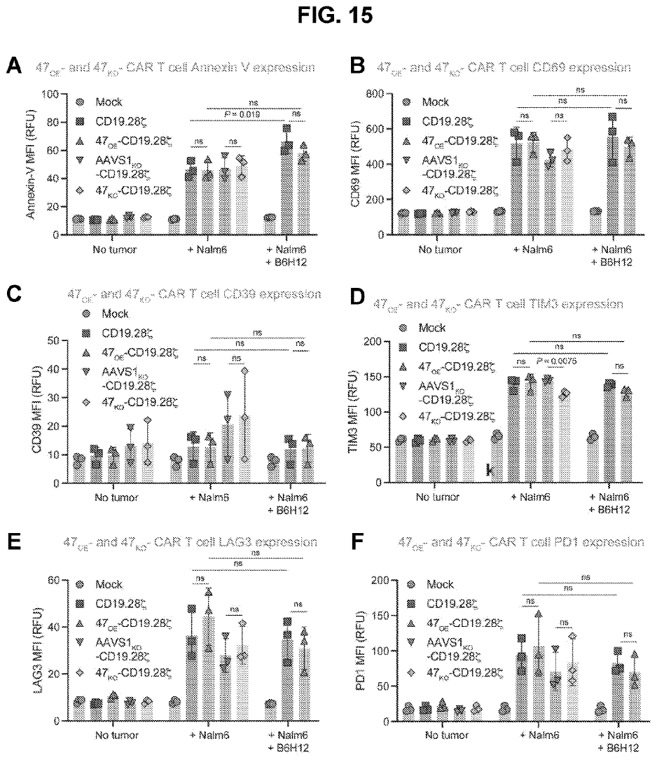

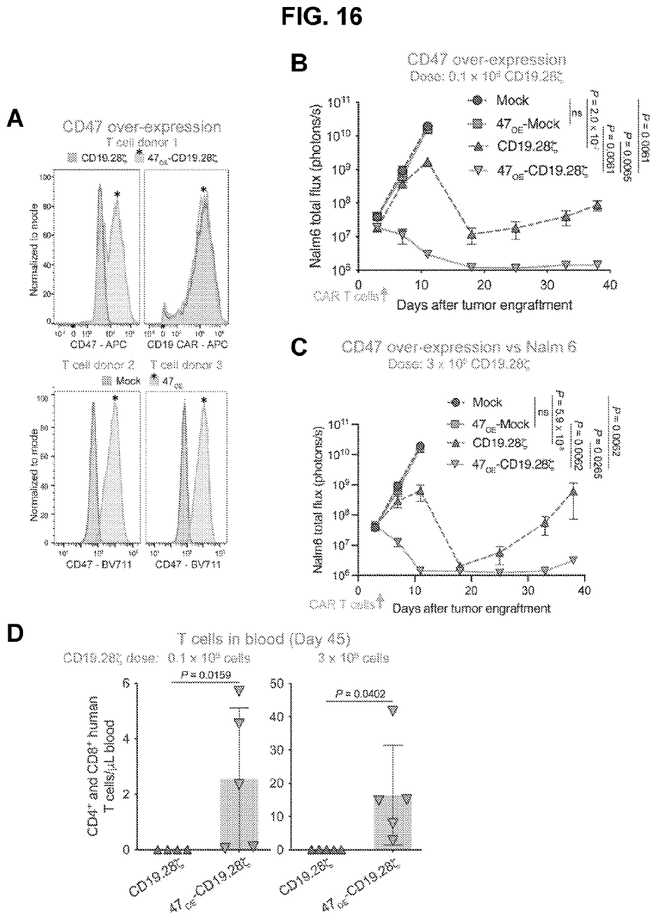

Given the critical importance of CD47 expression for T cell persistence in vivo, overexpression of CD47 (47_{OE}), a technique that has been reported to prevent immune rejection by allogeneic cells⁴⁶, was interrogated for the ability to enhance CAR T cell persistence and efficacy in lymphopenic NSG mice, where immune rejection does not occur due to profound immune suppression. It was determined that modulation of CD47 expression (either through 47_{KO} or 47_{OE}) or addition of αCD47 did not alter CAR T cell expansion (FIG. 14A), viability (FIG. 14B), tumor killing (FIG. 14C-D), cytokine secretion (FIG. 14E-F), or expression of activation and exhaustion markers (FIG. 15A-F) in vitro. However, 47_{OE}-CD19.28ζ-CAR T mediated significantly better long-term antitumor efficacy in vivo compared to control CAR T cells (FIG. 16A-C), and improved T cell persistence (FIG. 16D). Thus, CD47 overexpression enhances CAR T persistence and efficacy, even in settings where immune rejection does not occur. Together these data demonstrate that survival of adoptively transferred T cells requires CD47 expression and SIRPα engagement and that CD47 expression density by T cells substantially impacts survival and antitumor potency of T cells, even when CD47 blocking agents are not administered and independent of the previously reported effects of CD47 on allograft rejection⁴⁶.

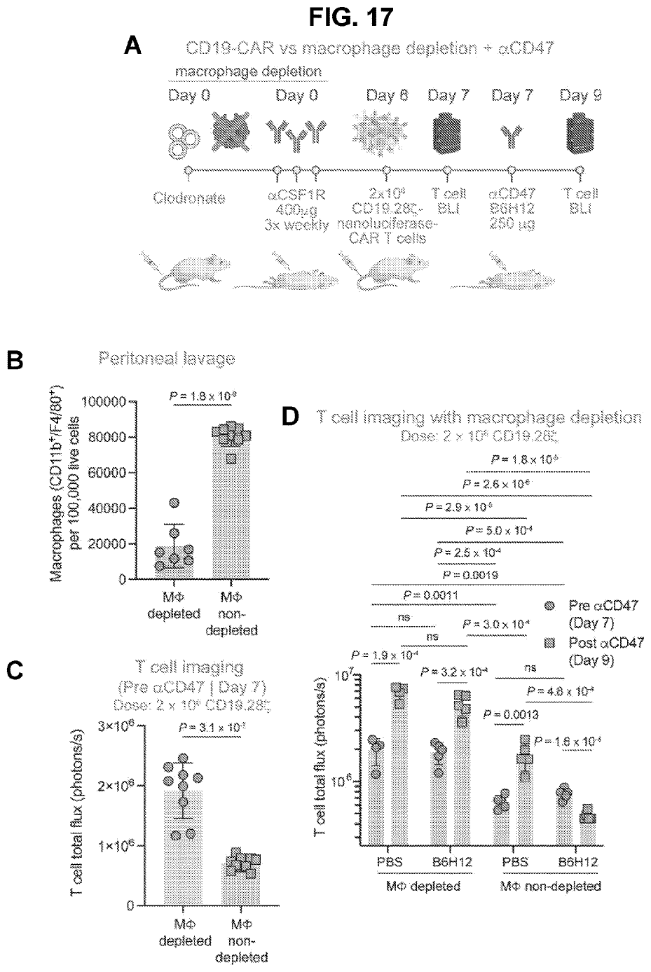

Based upon evidence that CD47 blockade enhances macrophage phagocytosis of tumors^{14,47,48}, it was queried whether macrophage mediated T cell depletion induced by αCD47 was responsible for the findings described above. Mice were treated with clodronate plus αCSF1R mAb to deplete macrophages³⁹ (FIG. 17A-B), then received CD19.28ζ-nLuc-CAR T cells±B6H12. On Day 7, one day following adoptive transfer but prior to B6H12 administration, BLI revealed significantly higher T cell numbers in mice with depleted macrophages consistent with macrophage mediated depletion of T cells even in the absence of αCD47 (FIG. 17C-D). Upon B6H12 administration, BLI revealed no loss of CAR T signal in macrophage depleted mice, compared to significantly reduced T cell BLI signal in mice with an intact macrophage compartment (FIG. 17D). Together, these results identify macrophages as barriers to engraftment and antitumor efficacy of adoptively transferred T cells and demonstrate an essential requirement for adequate levels of CD47 on T cells to engage SIRPα, even in hosts incapable of recognizing allogeneic disparities. They further explain the futility of combining αCD47 with adoptive T cell therapy and implicate macrophage mediated phagocytosis as an important regulator of T cell persistence in vivo.

### Example 3—Human CAR T Cells are Robustly Phagocytosed by Human Macrophages In Vitro, and Clinical Data Provides Evidence of Macrophage Mediated Phagocytosis of CAR T Cells

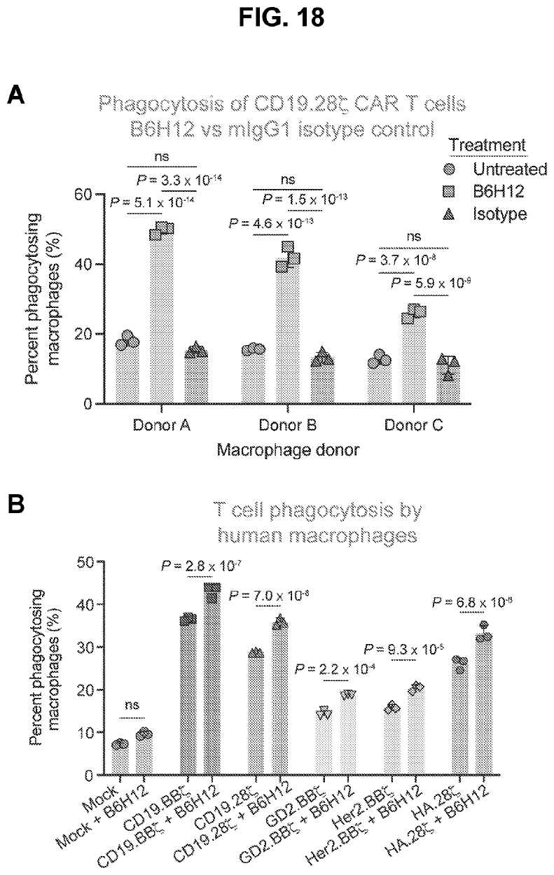

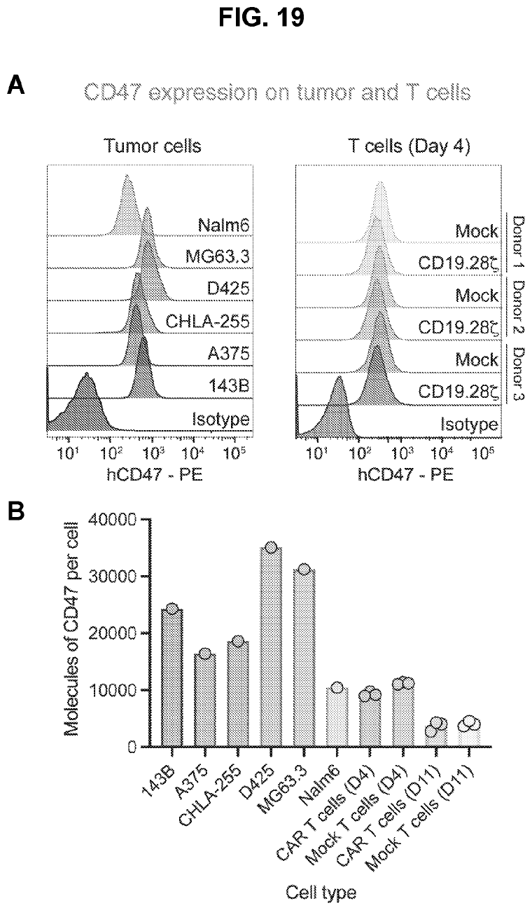

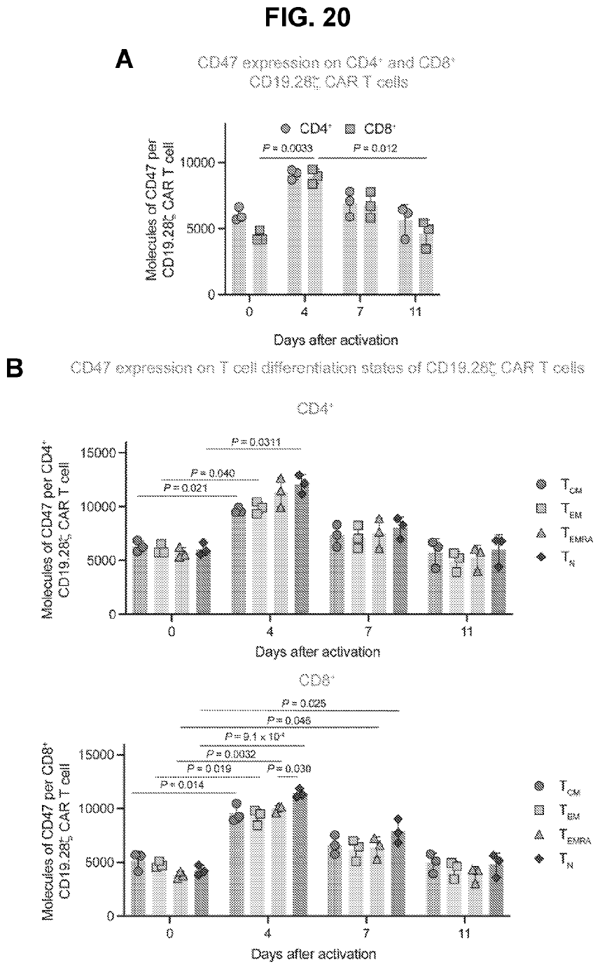

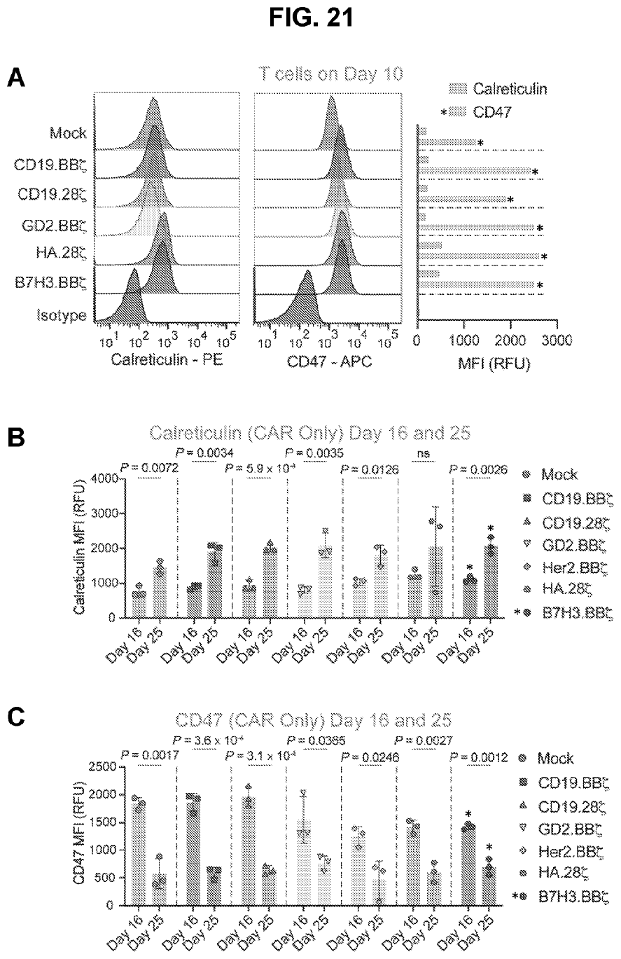

Next, the potential for primary human macrophages to phagocytose primary human T cells in vitro was investigated. Low levels of macrophage phagocytosis of mock transduced T cells were observed at baseline, while T cells transduced to express CARs were phagocytosed at significantly higher levels (FIG. 18A-B), which was further increased after specific addition of B6H12 (FIG. 18A). Macrophage phagocytosis is regulated by a balance of “eat me” signals, such as calreticulin⁴⁸, and “don't eat me” signals, such as CD47. Flow cytometry revealed that CAR T cells expressed fewer CD47 molecules than the tumor lines used in this study (FIG. 19A-B), consistent with a model wherein CAR T cells present limiting “don't eat me” signals to macrophages. CD47 expression was relatively uniform between T cell subtypes, including CD4⁺ and CD8⁺ cells (FIG. 20A), as well as among T cell differentiation states (FIG. 20B), however calreticulin expression was observed to increase over time on CAR T cells in culture (FIG. 21A-B), while CD47 expression decreased during the same period (FIG. 21C). Of note, CD47 expression on CAR T cells of all subtypes was highest immediately after anti-CD3/CD28 bead activation, returning to pre-activation levels after one week in culture (FIG. 20B), consistent with a model wherein aged CAR T cells are more susceptible to phagocytosis.

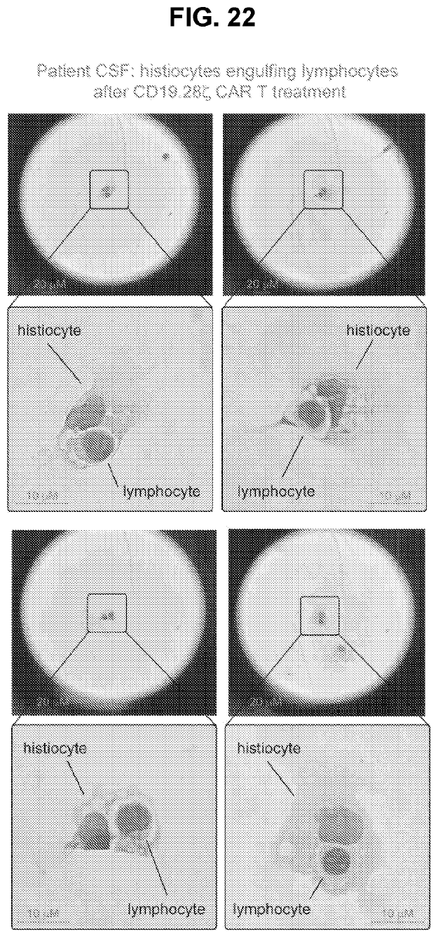

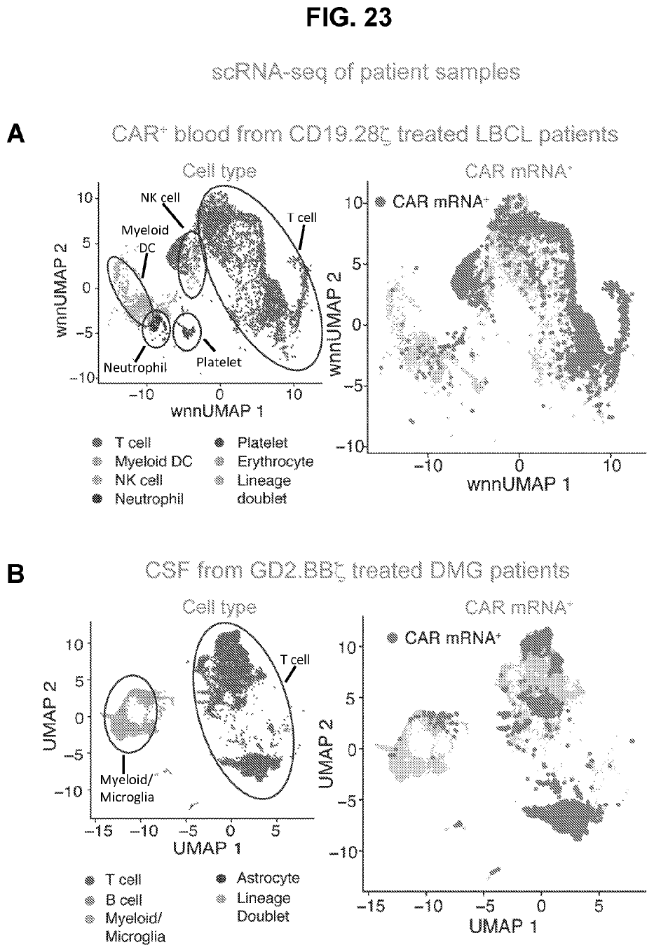

During the course of these experiments, routine cytologic analysis of cerebrospinal fluid collected from a patient treated with axicabtagene ciloleucel (axi-cel), a commercial CD19.28ζ-CAR T cell therapy²⁹, revealed histiocytes engulfing lymphocytes (FIG. 22), consistent with macrophage mediated phagocytosis^{49,50}. To address this possibility more systematically, single-cell RNA sequencing (scRNA-seq) data collected from two recent clinical studies that enrolled patients treated with axi-ce1⁵¹ and GD2.BBζ-CAR T cells⁵², respectively, was analyzed. Both datasets provided clear evidence for CAR mRNA in myeloid cells, consistent with macrophage mediated phagocytosis of CAR T cells in humans (FIG. 23A-B). These data provide further evidence in support of a model wherein myeloid cells phagocytose CAR T cells, and thus may limit durable engraftment of adoptively transferred cells or survival of activated T cells in clinical settings.

### Example 4—an Engineered Variant of CD47 with Selective Binding Retains “Don't Eat Me” Function while Allowing for Escape from CD47 Blockade

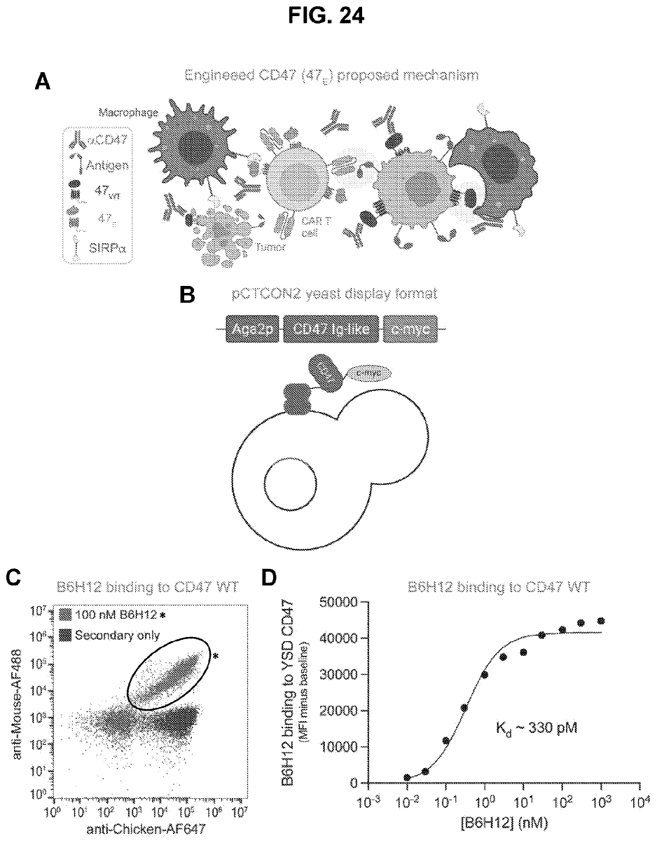

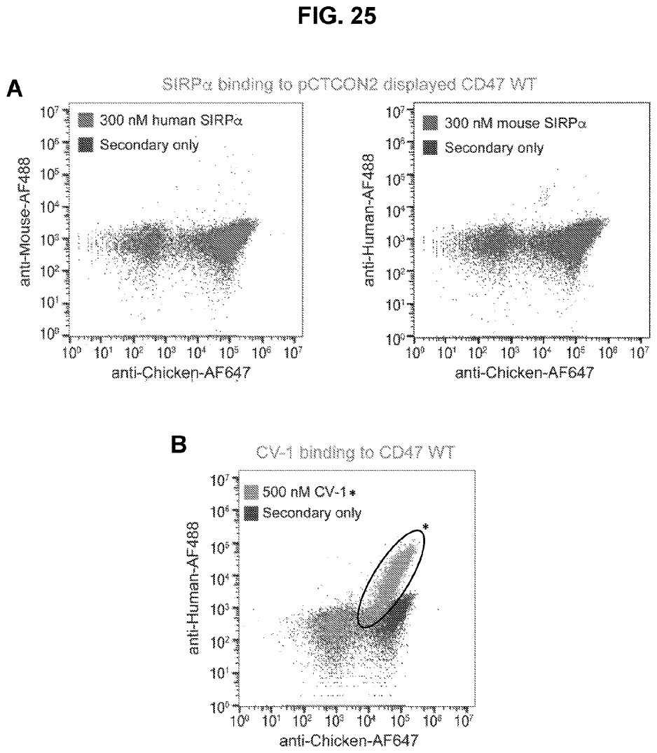

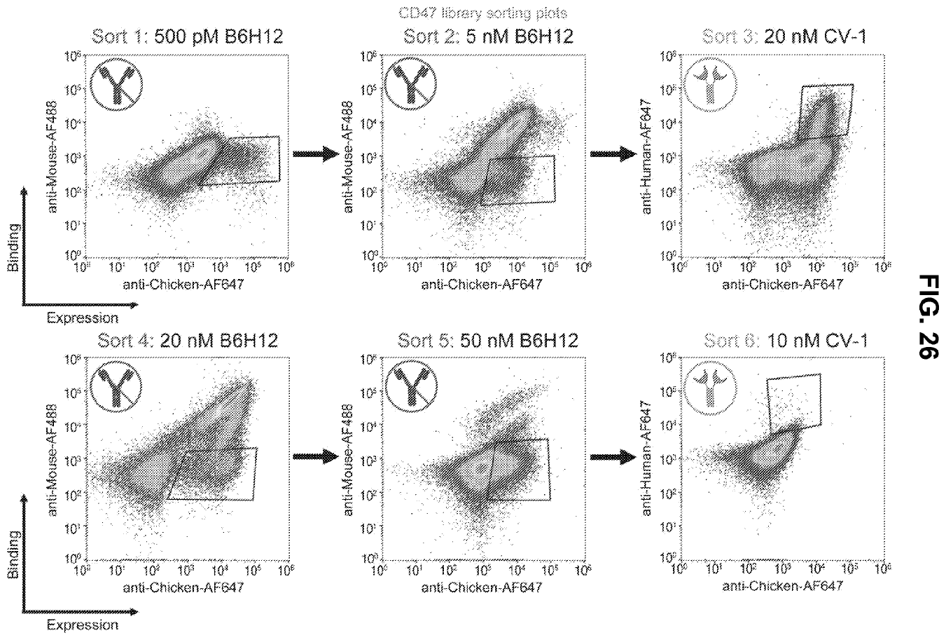

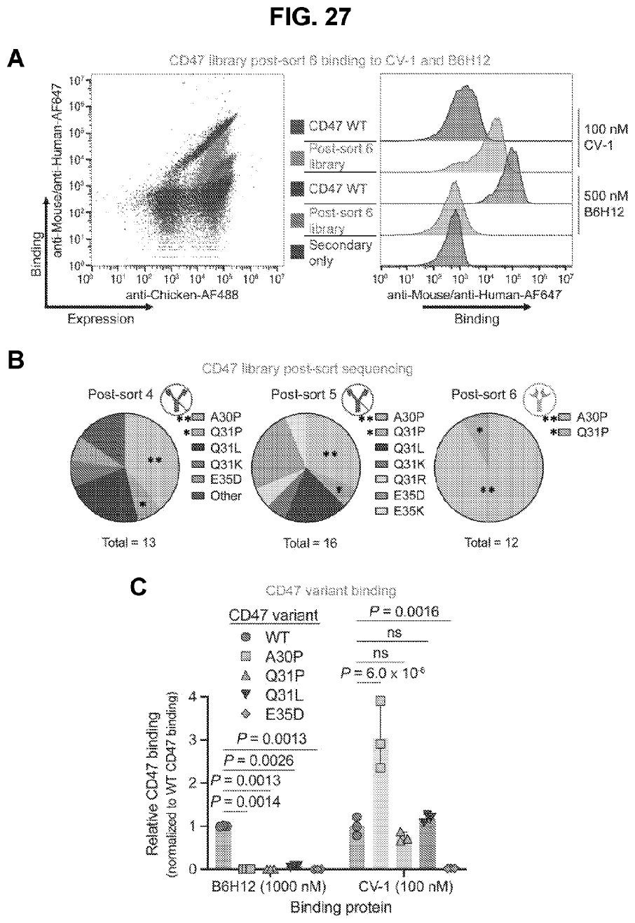

To induce selective tumor phagocytosis via CD47 blockade while protecting T cells from phagocytosis, a CD47 variant with mutations that ablate αCD47 binding while retaining binding to SIRPα (FIG. 24A) was engineered. To do so, the CD47 Ig-like domain was displayed on the surface of yeast and strong binding to B6H12, but not SIRPα was detected (FIG. 24B-C, FIG. 25A), as had been reported previously and attributed to the lack of a free N-terminus on CD47 when displayed on yeast⁵³. As a proxy for SIRPα binding, the engineered SIRPα variant, CV-1⁴⁵ was used (FIG. 25B). A library of yeast displayed CD47 mutant variants was subjected to six total successive FACS sorts, alternating between negative sorts against B6H12 and positive sorts towards CV-1 (FIG. 26), resulting in a bulk library population of CD47 variants after the final sort that retained binding to CV-1 but demonstrated near complete loss of binding to B6H12 (FIG. 27A). Sequencing revealed that all variants identified in the final sort contained a single A30P or Q31P point mutation (FIG. 27B), both of which localize to the BC loop of CD47. When displayed as individual CD47 variants on yeast, both A30P (47_{A30P}) and Q31P (47_{Q31P}) mutations manifested no binding to 1 μM B6H12 but similar or even enhanced binding to CV-1 (FIG. 27C).

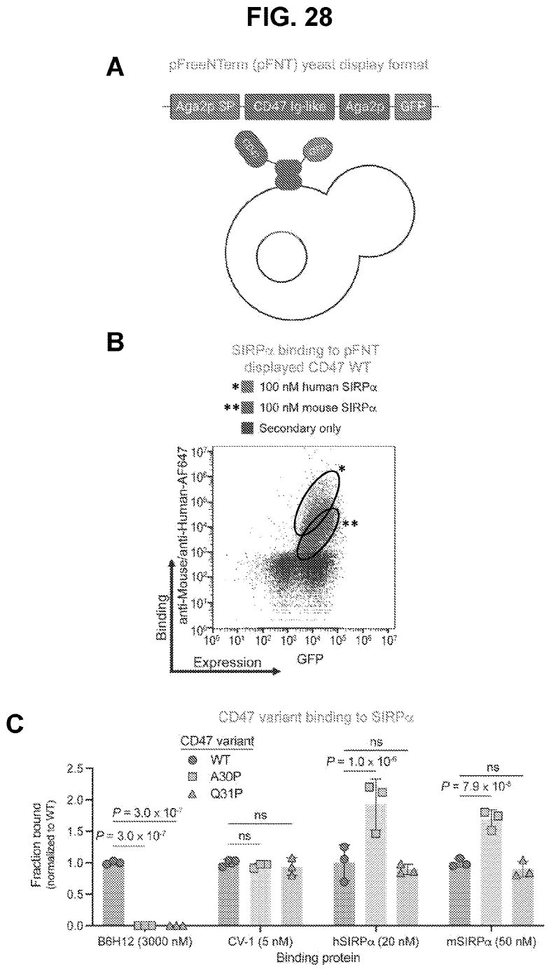

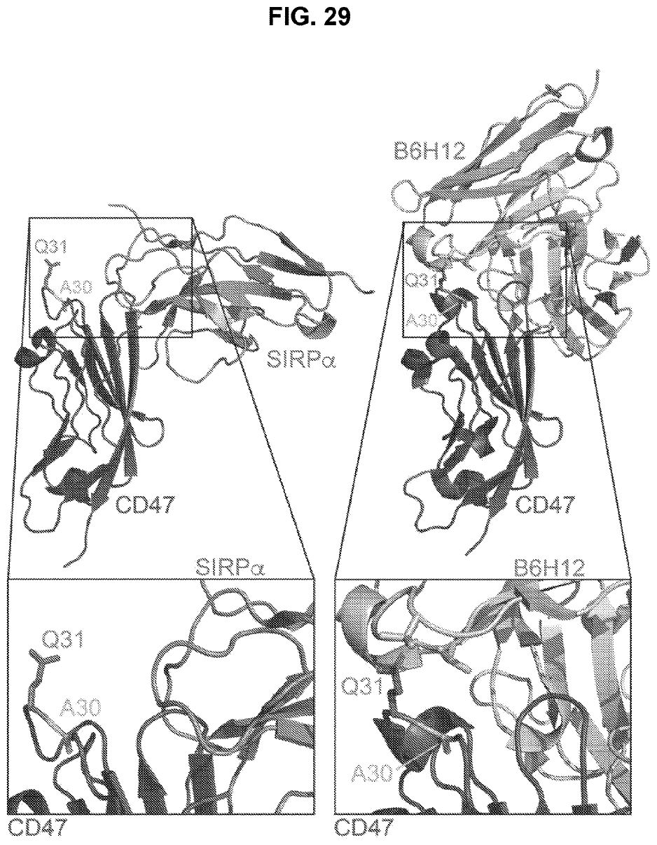

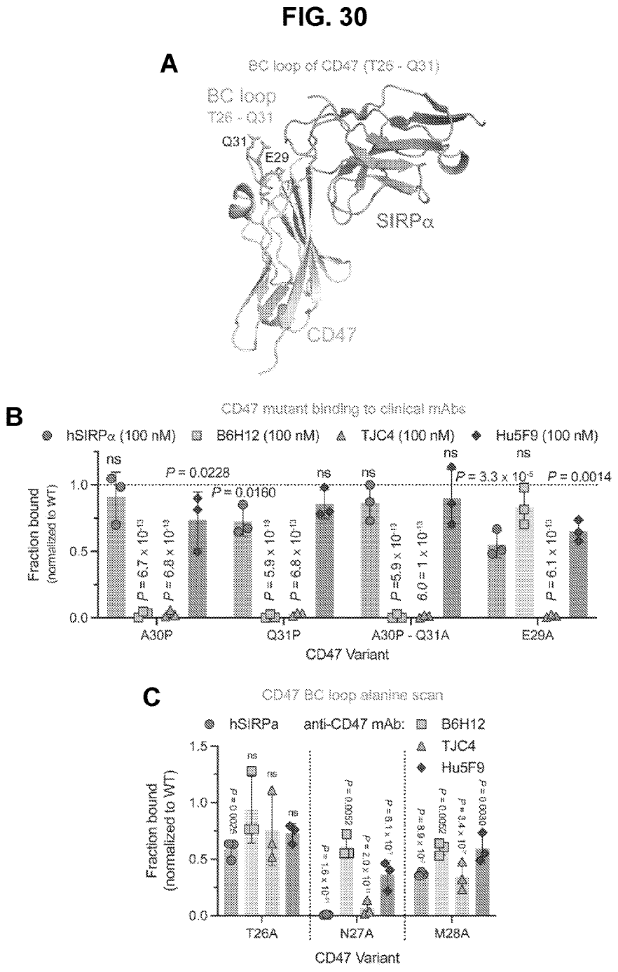

To assess binding to wild-type (WT) SIRPα, 47_{A30P} and 47_{Q31P} mutants were next displayed on yeast in a more natural orientation, with a free CD47 N-terminus⁵³ (FIG. 28A-B). Both mutants retained binding to mouse and human SIRPα but demonstrated no binding to B6H12, even at the high concentration of 3 μM (FIG. 28C). These results are consistent with current structural understanding of CD47-SIRPα interactions, whereby SIRPα predominantly contacts CD47 through residues in the CD47 FG loop and N-terminus⁵⁴ (FIG. 29), while forming more minor contacts with the CD47 BC loop, which encompasses T26-Q3154 (FIG. 29, FIG. 30A), potentially leaving residues in this region amenable to mutation. Because the CD47 BC loop lies near the critical CD47 FG loop, it can serve as an anchoring point for αCD47 blocking mAbs like B6H12^{55,56}, with the Q31 residue appearing particularly important for antibody binding^{55,56} (FIG. 29).

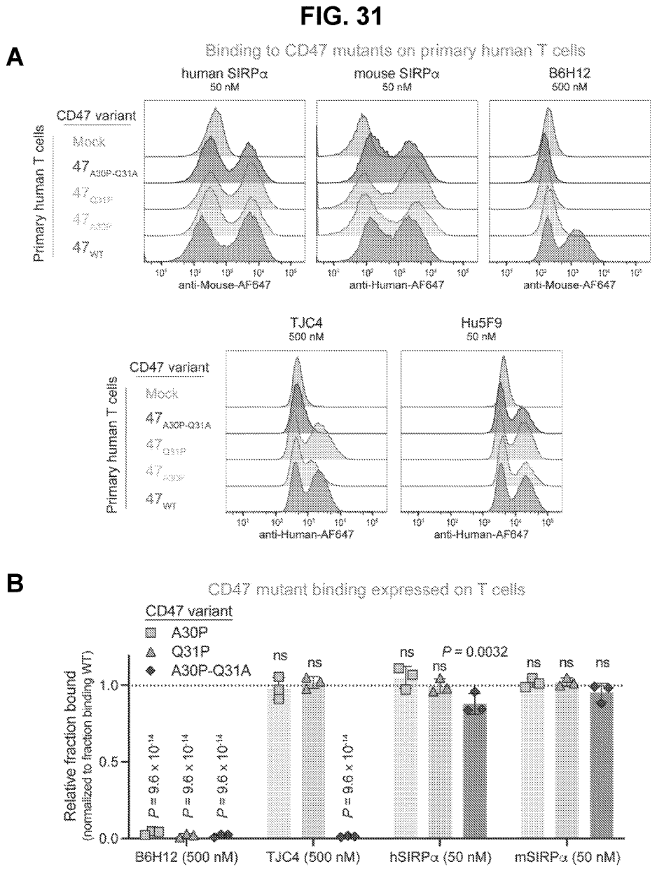

To determine whether the generated CD47 mutants evaded binding by CD47 blocking mAbs currently in clinical trials, binding of TJC4^{57,58} (lemzoparlimab; Phase Ill) and Hu5F9¹⁷ (magrolimab; Phase III) to yeast displayed CD47 mutants was analyzed. We began by performing an alanine scan of the entire BC loop (T26-Q31), comparing binding to human SIRPα, B6H12, TJC4, and Hu5F9 (FIG. 30B-C). Most mutations allowed for some retained SIRPα binding, with mutations to A30 or Q31 manifesting the most minimal impact on SIRPα binding. Hu5F9, which has a binding footprint that largely overlaps with SIRPα⁵⁹, demonstrated minimal loss of binding to any of the BC loop mutants, including 47_{A30P} or 47_{Q31P}. However, TJC4, which structurally binds CD47 similarly to B6H12⁶⁰, no longer bound 47_{A30P}, 47_{Q31P}, and 47_{A30P-Q31A}, nor did it bind 47_{E29A}, which manifests an additional mutation that did not affect B6H12 binding (FIG. 30B-C). Next, binding of SIRPα, B6H12 and TJC4 to full-length 47_{WT}, 47_{A30P}, 47_{Q31P}, and 47_{A30P-Q31A}, expressed on primary human T cells, was profiled. Binding of both human and mouse SIRPα was largely unaffected by any of the three mutants indicating that these variants are predicted to retain their function as “don't eat me” signals (FIG. 31A-B). However, no B6H12 binding over baseline to any of the three mutants was detected, and TJC4 binding was completely ablated by the 47_{A30P-Q31A} double mutant. These data demonstrate that CD47 mutations to the BC loop, and A30 and Q31 specifically, generate proteins that retain SIRPα binding but are exempt from binding to multiple CD47 mAbs, providing proof-of-concept for the ability to engineer “don't eat me” signaling CD47 variants that will not be blocked by αCD47 mAbs, which is predicted to drive tumor specific phagocytosis while sparing T cells in the TME.

### Example 5—Expression of Engineered CD47 Protects T Cells from Anti-CD47 Mediated Phagocytosis and Allows for Enhanced Anti-Tumor Efficacy Through Paired Immunotherapy

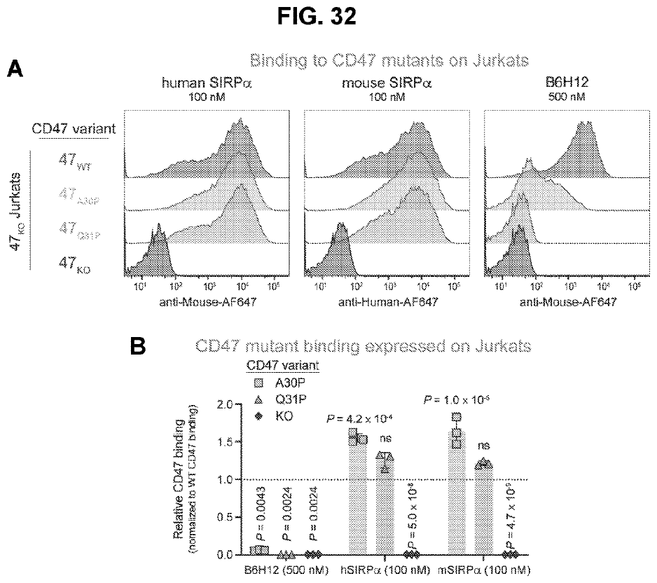

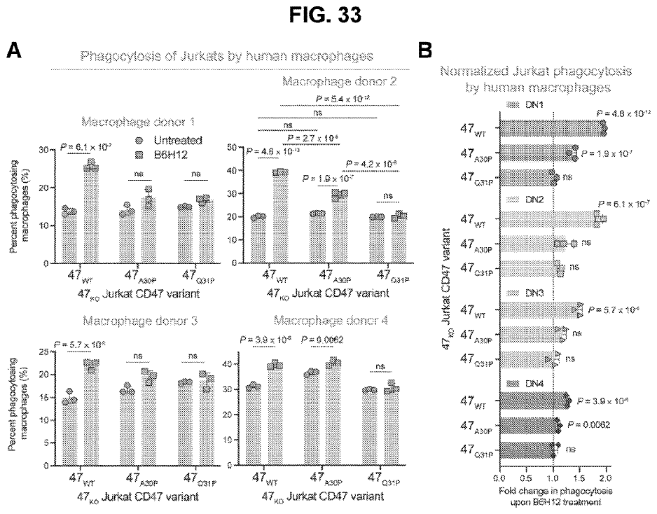

Next, phagocytosis of 47_{KO} Jurkat cells engineered to express either 47_{WT}, 47_{A30P}, or 47_{Q31P} by human donor macrophages was measured. CD47 mutants expressed on Jurkats demonstrated similar binding properties to αCD47 mAbs and SIRPα as observed on primary T cells, with 47_{Q31P} leading to the greatest loss of B6H12 binding (FIG. 32A-B). Across multiple macrophage donors, expression of either mutant significantly reduced phagocytosis after B6H12 incubation (FIG. 33A-B), but 47_{A30P} provided less protection compared to 47_{Q31P}, which completely prevented additional phagocytosis after incubation with B6H12. Based on this promising profile, 47_{Q31P}, referred to as 47_{E} (“engineered CD47”), was chosen for further study.

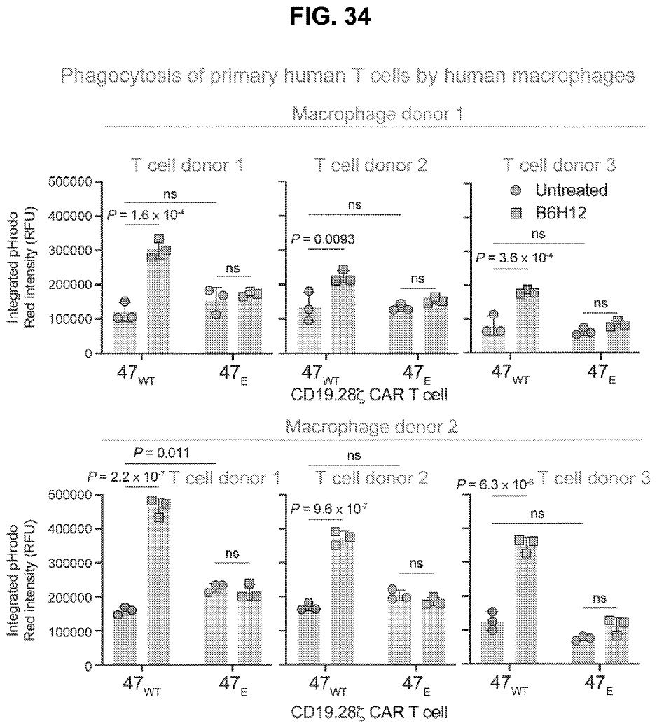

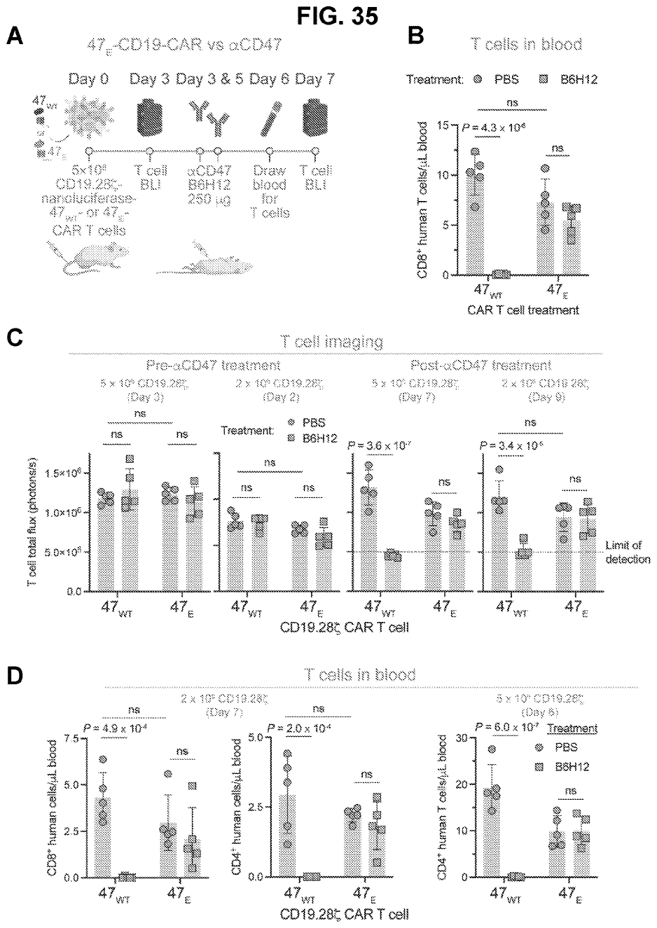

To study the effects of 47_{E} in human T cells, endogenous CD47 was knocked out using CRISPR/Cas9, then CAR, TCR, and/or 47_{E} or wild-type CD47 (47_{WT}) were retrovirally introduced, before phagocytosis was measured in vitro and in vivo in the presence of B6H12. Demonstrated using an Incucyte phagocytosis assay across multiple T cell and macrophage donors, B6H12 treatment did not enhance phagocytosis of 47_{E}-CD19.28ζ CAR T cells, in contrast to control 47_{WT}-CD19.28ζ CAR T cells (FIG. 34). To assess T cell depletion in vivo, 47_{WT}- or 47_{E}-CD19.28ζ-nLuc CAR T cells were administered to non-tumor bearing mice (FIG. 35A). Similar levels of T cells were observed via BLI across groups prior to B6H12 administration (FIG. 35C). After B6H12 treatment, 47_{WT}-CAR T were completely depleted, in contrast to 47_{E}-CAR T cells which persisted at similar levels compared to mice that had only received PBS vehicle control (FIG. 35B-D). These data demonstrate that 47_{E} functions as a “don't eat me” signal in vitro and in vivo, but remains inert from binding B6H12, and thereby protects T cells from additional phagocytosis after CD47 blockade.

### Example 6—CAR T Cell Treatment Leads to Macrophage Tumor Infiltration which is Augmented in Animals Treated with 47_{E}-CAR T Cells Plus αCD47

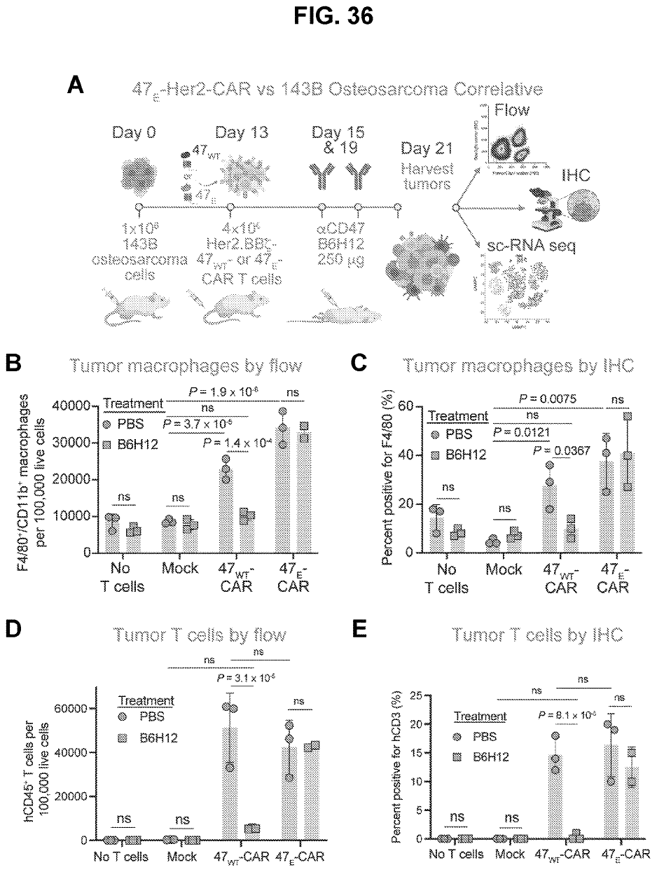

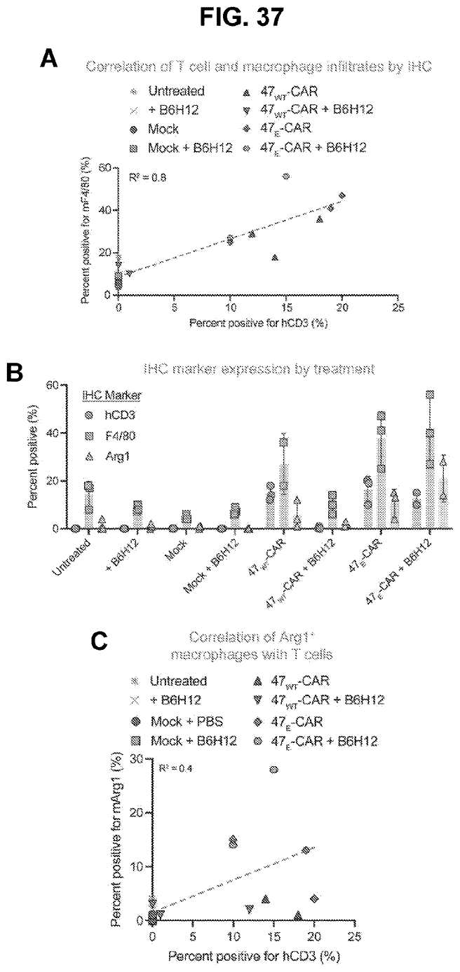

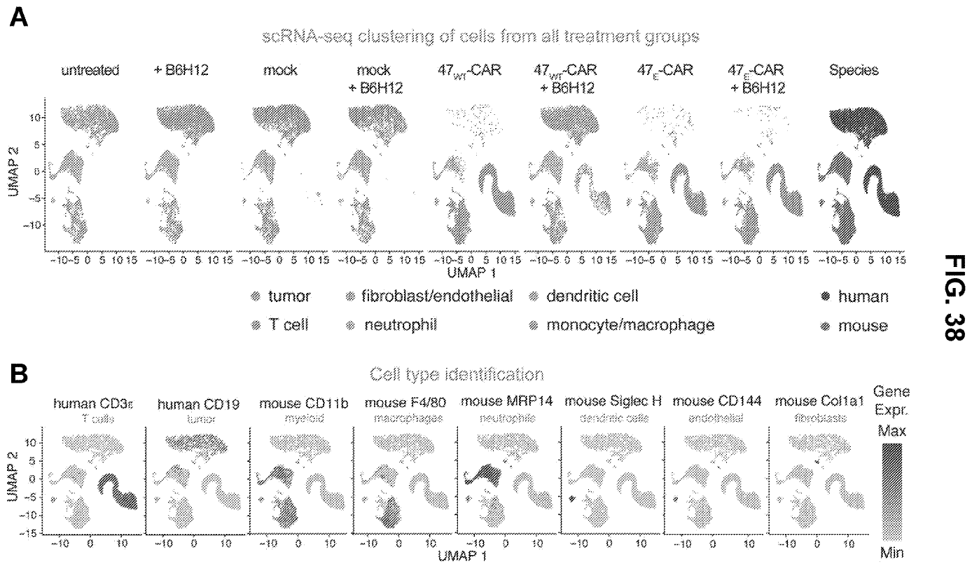

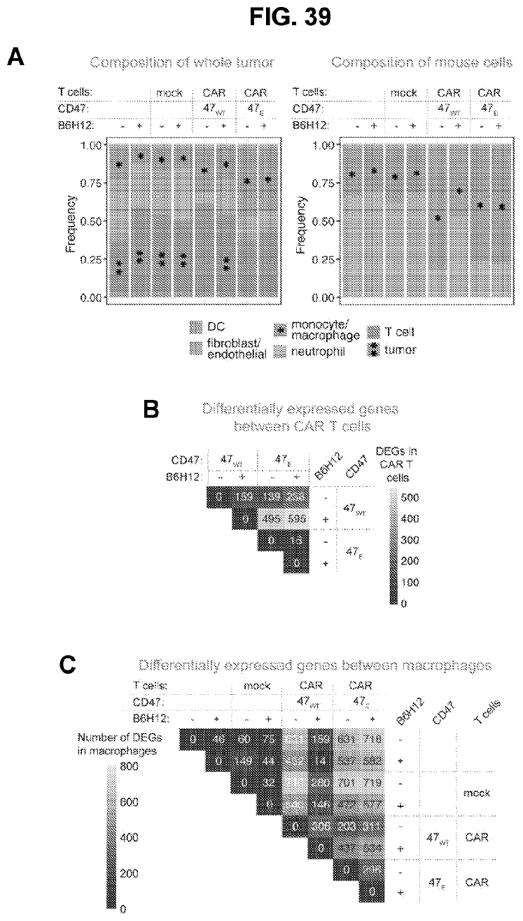

To profile the effects of combination therapy on the TME of 143B osteosarcoma, tumor bearing mice were treated with no T cells, mock, 47_{WT}- or 47_{E}-Her2.BBζ-CAR T±B6H12 (FIG. 36A). After 8 days, tumors from CAR T recipients demonstrated significant increases in F4/80⁺ murine macrophages, compared with mock or untreated animals (FIG. 36B-C). B6H12 treatment did not significantly impact macrophage levels in the TME of 47_{E}-CAR T recipients, whereas macrophages were substantially reduced following B6H12 therapy in 47_{WT}-CAR T recipients (FIG. 36B-C), presumably due to T cell depletion (FIG. 36D-E). CAR T and macrophage infiltration into tumors were highly correlated, consistent with a model whereby CAR T cells recruit macrophages into the tumor, and macrophage persistence is dependent upon CAR T persistence in the tumor (FIG. 37A-C). ScRNA-seq analysis profiling of both human (tumor and T cells) and mouse (immune and fibroblast) cells in dissociated tumors (FIG. 38A-B) confirmed CAR T mediated increases in the frequencies of macrophages within the tumor, which was ablated in 47_{WT}-CAR T recipients following B6H12 treatment and subsequent T cell depletion, but persisted in 47_{E}-CAR T recipients (FIG. 39A).

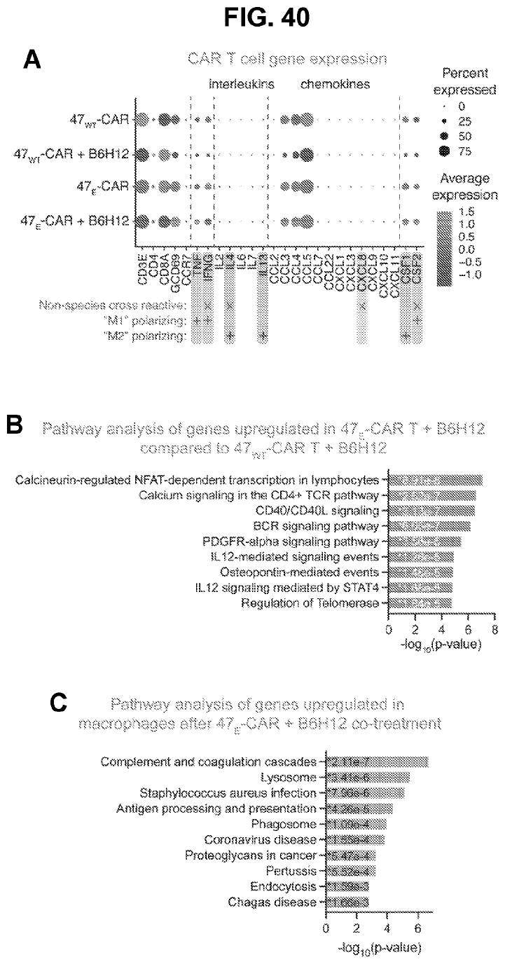

Next genes potentially responsible for macrophage recruitment and activation following CAR T cell therapy in this model were identified. 47_{WT}- and 47_{E}-CAR T recipients demonstrated robust T cell expression of TNFα, IFN-γ, CCL3, CCL4, and CCL5, CSF1 (M-SCF), and CSF2 (GM-CSF) (FIG. 40A), which collectively attract and activate monocytes and macrophages^{61,62} and have been implicated in T cell mediated macrophage recruitment into tumors⁶³. Of note, because IFN-γ and CSF2, are not species cross-reactive⁶⁴, the full potential of T cell-macrophage crosstalk in this model is likely underestimated. 47_{E}-CAR T gene expression was essentially the same±B6H12 therapy, however T cells in the TME of B6H12 recipients co-treated with 47_{E}-CAR T showed 595 differentially expressed genes (DEGs) compared to those co-treated with 47_{WT}-CAR T (FIG. 39B), including gene sets associated with IL-12 signaling⁶⁵ and CD40/CD40L signaling^{66,67} (FIG. 40B). These data provide evidence for substantial crosstalk between myeloid cells and T cells within the 47_{E}-CAR T TME which is lacking in the TME of 47_{WT}-CAR T recipients after B6H12 co-treatment.

DEG analyses in the major macrophage cluster across treatments showed that treatment with 47_{WT}-CAR T alone induced 621 DEGs in macrophages compared to the untreated condition, and this effect was magnified following 47_{E}-CAR T co-treatment with B6H12, with 718 DEGs (FIG. 39C). Interestingly, the effect of B6H12 therapy on macrophage gene expression when administered as a single agent was minimal (46 DEGs) (FIG. 39C). However, B6H12 co-administration with 47_{WT}-CAR T dramatically reduced macrophage DEGs, likely due to CAR T depletion (FIG. 39C). Pathway analysis of genes upregulated by 47_{E}-CAR T plus B6H12 highlighted macrophage activation indicated by enrichment of lysosome, complement, antigen presentation, and phagosome pathways^{68,69} (FIG. 40C). Together, these results demonstrate a feed forward loop wherein CAR T cells drive recruitment and activation of macrophages within the TME and simultaneously, macrophages enhance activating pathways in 47_{E}-CAR T cells in the TME. These effects do not occur within the 47_{WT}-CAR T TME, wherein CAR T depletion abrogates the cycle after CD47 blockade.

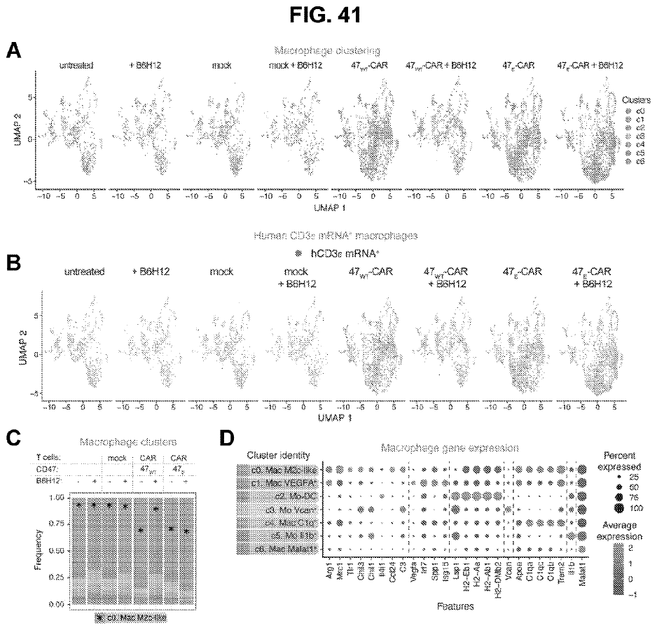

To further characterize changes in the macrophage compartment induced by 47_{E}-CAR T, the macrophage/monocyte cluster was re-clustered (FIG. 41A). As observed in clinical data (FIG. 23), numerous macrophages that contained hCD3s mRNA within multiple CAR T treated macrophage sub-clusters were identified (FIG. 41B), consistent with macrophage mediated phagocytosis of CAR T cells. Expansion of macrophage cluster “c0,” which was enriched following CAR T treatment and further expanded following 47_{E}-CAR T plus B6H12, but nearly completely absent after 47_{WT}-CAR T plus B6H12, was also observed (FIG. 41A,C), suggesting that these macrophages are dependent upon CAR T cell accumulation within the TME. Key DEGs in the expanded cluster were associated with M2c-like macrophages⁷⁰ (FIG. 41C-D). The M2c-like cluster (c0) highly expressed canonical M2 macrophage genes such as Arg1, Mrc1, and Chil3, in addition to M2c genes such as Tlr1⁷⁰ (FIG. 41D). While M2 macrophages are generally understood to be pro-tumorigenic, they have also been demonstrated to manifest strong phagocytic potential, especially those in M2c subclass^{71,72}. Together, these data demonstrate robust crosstalk between CAR T cells and macrophages in tumors, with significant dependency of the macrophage population on CAR T persistence in the TME, and macrophage induced induction of gene expression programs in the CAR T predicted to enhance antitumor effects.

### Example 7—47_{E}-CAR T Plus αCD47 Induces Enhanced Antitumor Efficacy

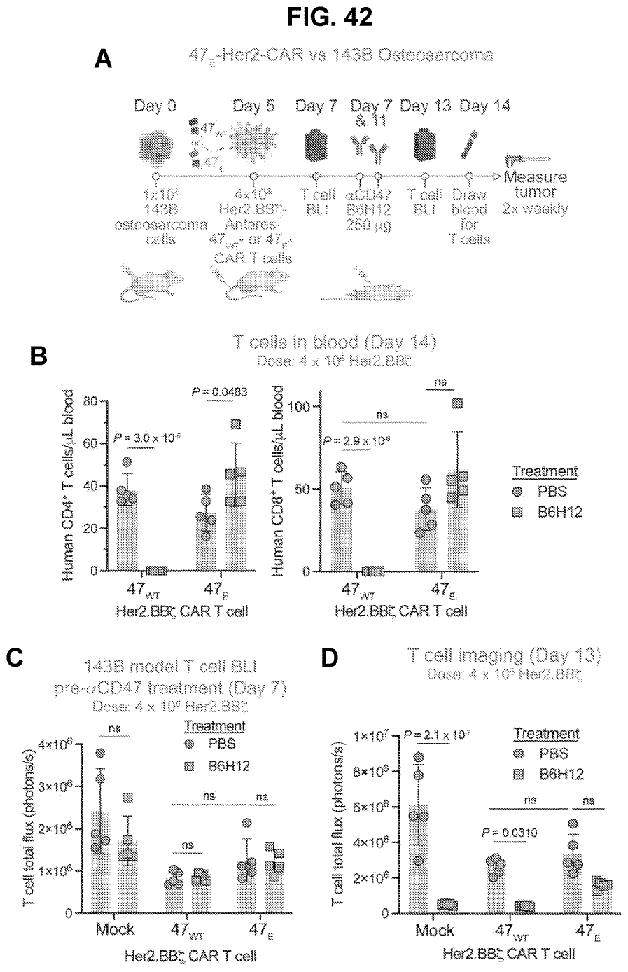

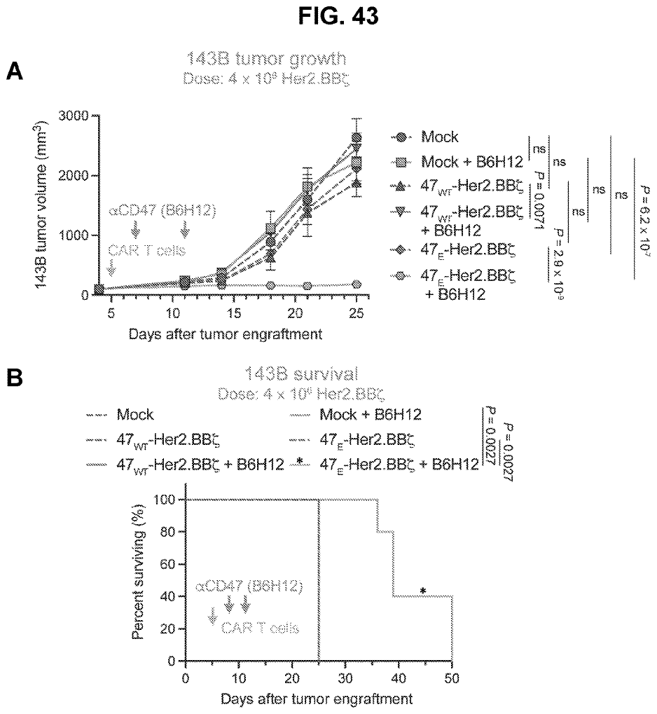

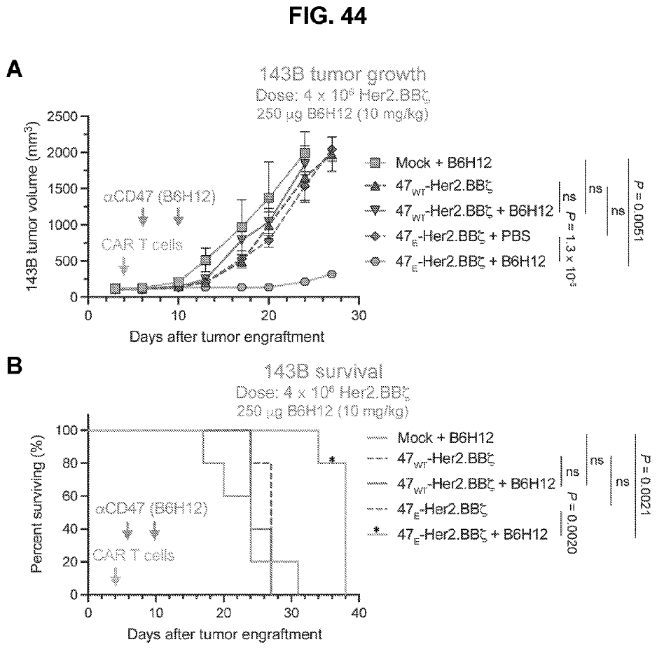

Next, the antitumor effects of combined 47_{WT}- vs 47_{E}-T cells plus αCD47 therapy were assessed in multiple tumor models treated with cell therapies, many of which had been shown previously to be intractable to standard combination of CAR T and αCD47 therapies. Antitumor effects of combination therapy were first interrogated in the aggressive orthotopic osteosarcoma model, 143B, where both CAR T and αCD47 therapy have minimal effect as monotherapies (FIG. 1B), but where striking T cell mediated recruitment of macrophages into tumors had been observed (FIG. 36B-C). Mice bearing 143B tumors in the tibia periosteum received 47_{WT}- or 47_{E}-Her2.BBζ-CAR T cells, B6H12 (FIG. 42A). All T cells also expressed Antares, a nanoluciferase-orange fluorescent protein fusion with enhanced sensitivity for BLI^{34,73}. Following B6H12 administration, 47_{WT}-CAR T were completely depleted, while 47_{E}-CAR T persisted over the course of multiple weeks (FIG. 42B-D). Over two independent experiments, no meaningful tumor control was observed after treatment with 47_{WT}-CAR T or 47_{E}-CAR T alone, or mock T cells paired with B6H12, while B6H12 combined with 47_{WT}-CAR T delivered no significant antitumor efficacy (FIG. 43A-B, FIG. 44A-B). However, strikingly, B6H12 plus 47_{E}-CAR T induced marked tumor control, a significant delay in tumor outgrowth, and improvement in overall survival (FIG. 43A-B, FIG. 44A-B).

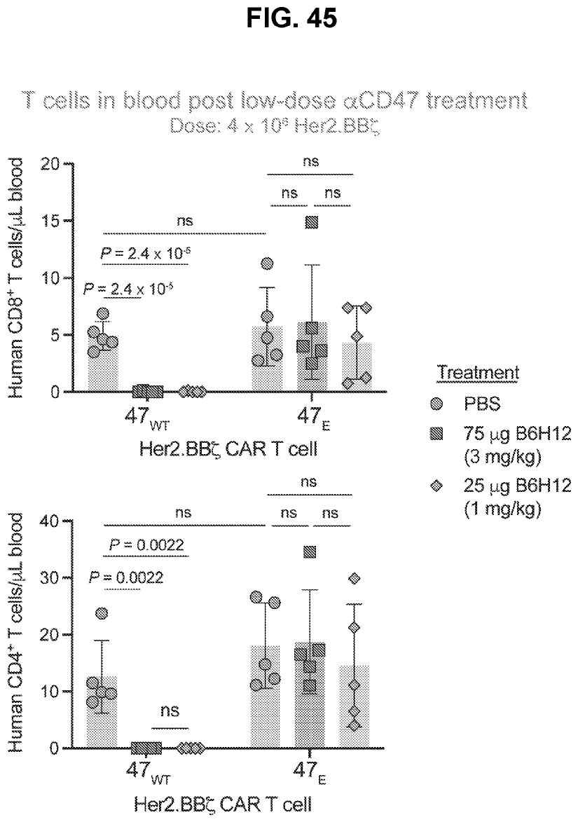

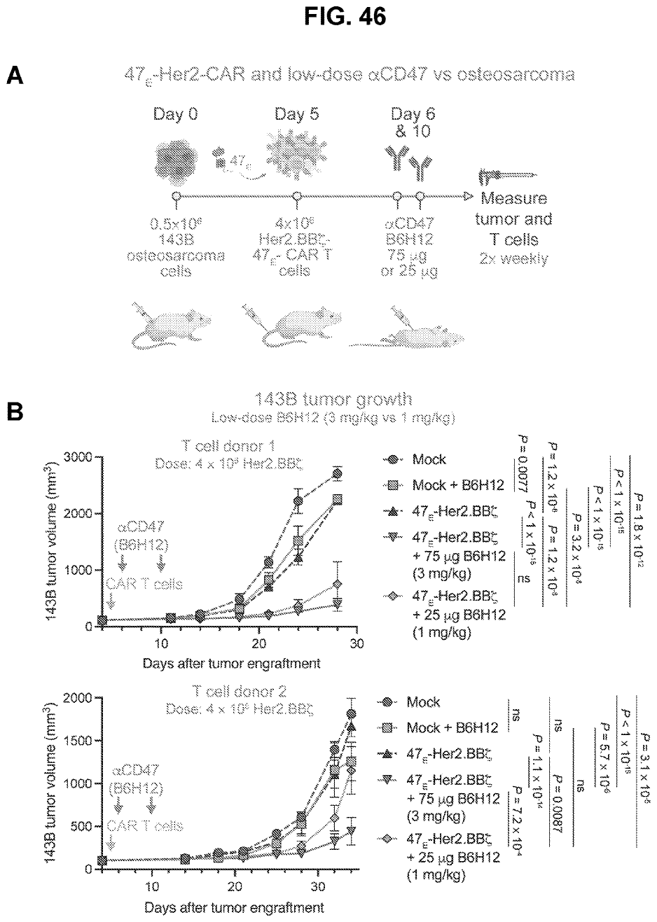

As αCD47 therapy is known to mediate toxicities at higher doses in the clinic¹⁵⁻¹⁷, whether low dose αCD47 might lead to antitumor efficacy in combination was interrogated, in an effort to potentially improve the safety profile of 47_{E}-CAR combination therapy. Lower doses of B6H12 induced marked depletion of 47_{WT}-Her2.BBζ-CAR T cells, but not 47_{E}-CAR T cells (FIG. 45), similar to depletion observed at higher B6H12 doses (FIG. 42D). In two independent experiments, co-treatment of 143B tumor bearing mice with 47_{E}-Her2.BBζ CAR T and low dose B6H12 resulted in significant, dose-dependent antitumor activity compared to either treatment alone (FIG. 46A-B), and on par with the efficacy observed upon treating with higher doses of B6H12 (FIG. 44, FIG. 45).

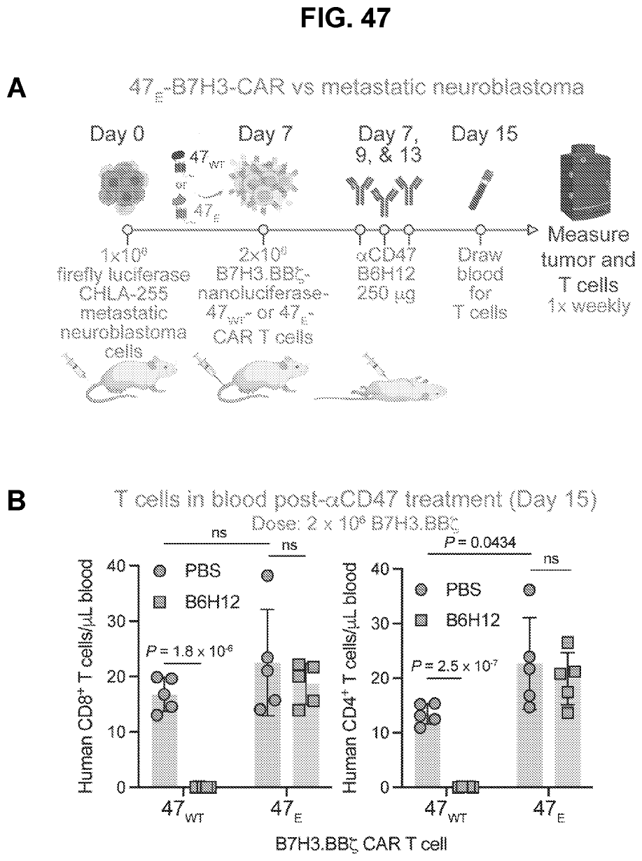

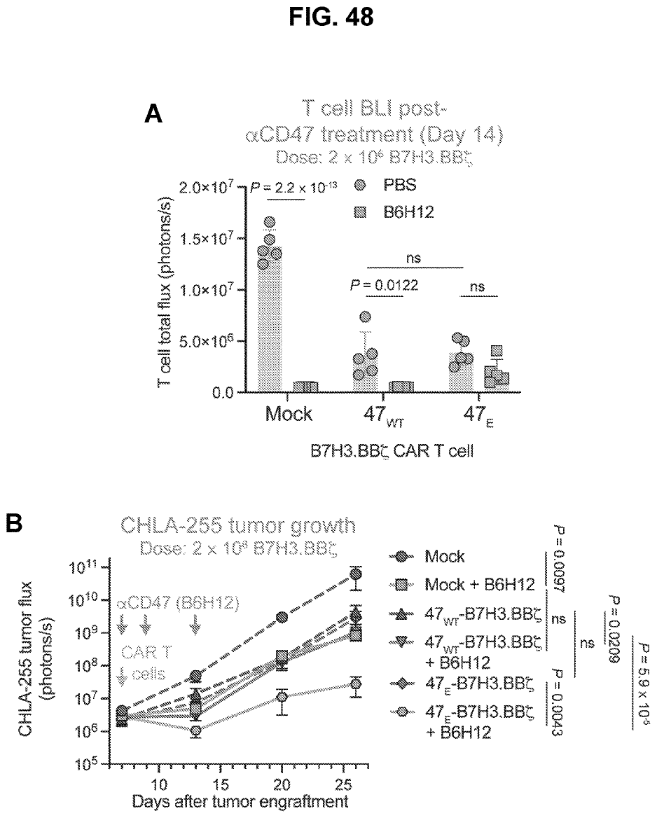

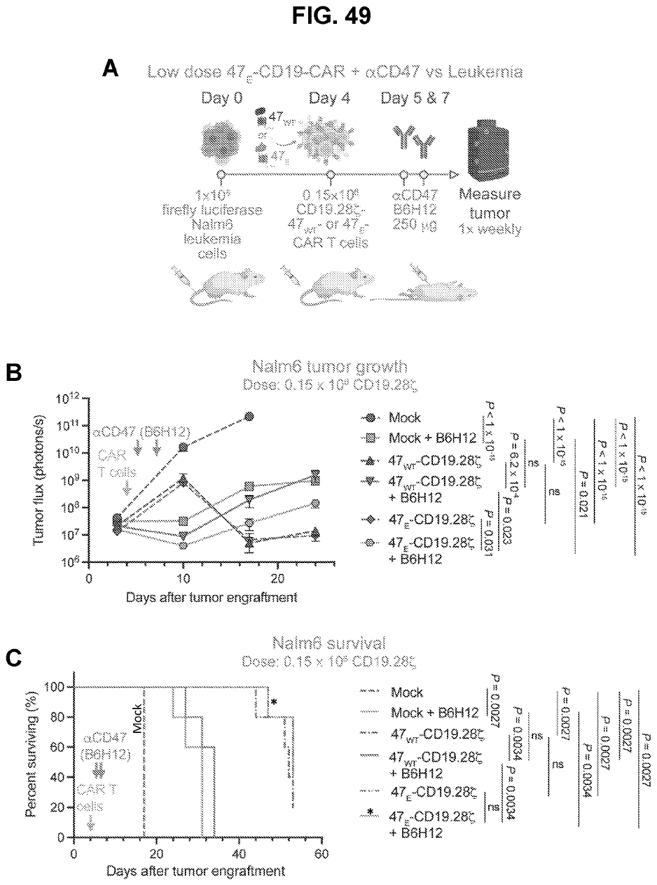

Next, pairing B6H12 with 47_{E}-CAR T in a metastatic neuroblastoma model was interrogated. Metastatic CHLA-255-fLuc bearing mice received 47_{WT}- or 47_{E}-B7H3.BBζ-nLuc-CAR T cells, ±B6H12. Persistence of 47_{E}-CAR T, but not 47_{WT}-CAR T (FIG. 47A-B, FIG. 48A) was observed. When B6H12 was administered, 47_{WT}-CAR T mediated no improvement in antitumor efficacy, while 47_{E}-CAR T mediated significantly improved antitumor efficacy, compared to either agent alone (FIG. 48B). Significantly enhanced antitumor efficacy was also observed after combination of B6H12 with a low dose of 47_{E}-CD19.28ζ CAR T cells compared to 47_{WT}-CD19.28ζ CAR T cells in treatment of Nalm6-fLuc tumor bearing mice (FIG. 49A-C).

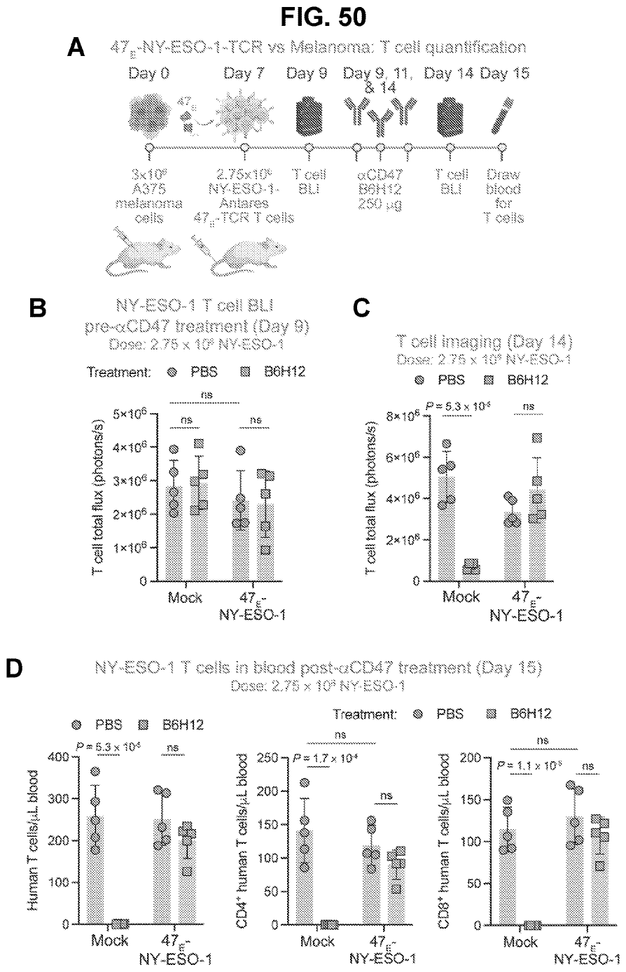

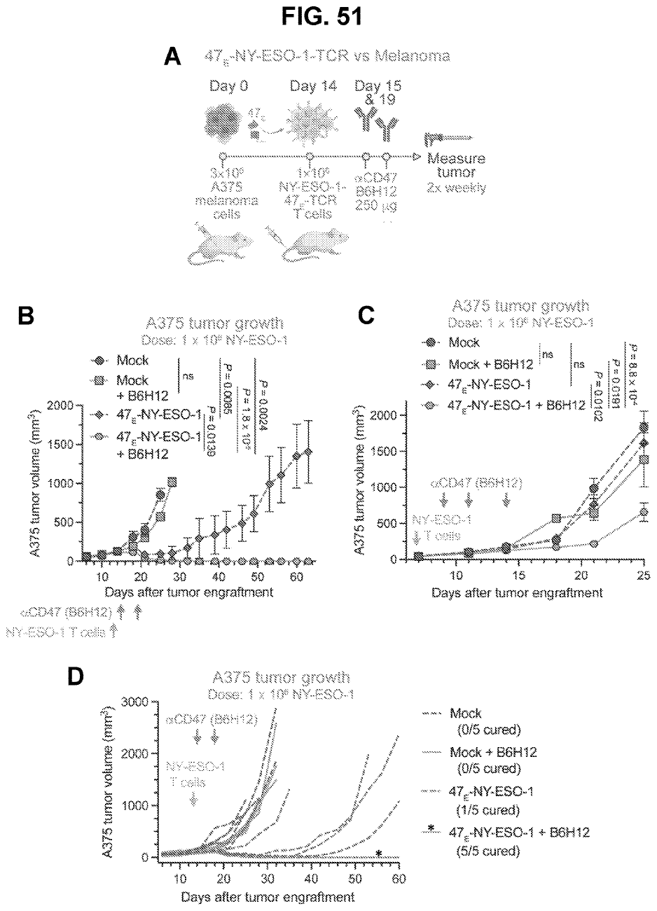

Finally, combination therapy with TCR-T therapy was investigated through administration of NY-ESO-1-TCR T cells to mice with A375 melanoma flank xenografts, using Antares expression to track T cell BLI (FIG. 50A). While 47_{WT}-NY-ESO-1 T cells were depleted and ineffective after B6H12 treatment (FIG. 6D), 47_{E}-NY-ESO-1 T cells were protected (FIG. 50B-D). A375 tumor growth was minimally slowed by B6H12 plus mock T cells and treatment with a low dose of 1×10⁶ 47_{E}-NY-ESO-1 T cells alone led to initial tumor control, but ultimate tumor outgrowth in 4/5 mice treated (FIG. 51A-B,D). By contrast, mice treated with 47_{E}-NY-ESO-1 T cells and B6H12 demonstrated complete tumor control and cure in 5/5 mice treated (FIG. 51B,D), and these results were confirmed using a second T cell donor (FIG. 51C).

Together, these results demonstrate strong synergy in solid, liquid, and metastatic tumors using CD47 blockade paired with 47_{E} expressed in therapeutic T cells, even at low doses and in conditions where both single-agent therapies showed no activity. The data illustrate that protection of CAR T cells from macrophage mediated phagocytosis results in a dramatic and sustained influx of macrophages within the TME, associated with T cell-macrophage crosstalk, and enhanced antitumor efficacy compared to treatment with either agent alone.

### DISCUSSION

Adoptive T cell therapy using chimeric antigen modified T cells (CAR T) has demonstrated success in treating hematologic malignancies¹⁹⁻²⁸, but less than 50% of patients treated with FDA approved CAR T experience durable disease control^{29,30} and CAR T cells have been less effective in treating solid tumors³¹, which make up most cancers³². Resistance to adoptive T cell therapies is attributed to multiple factors, including suppressive myeloid cells within the TME³³. The work presented here sought to enhance the efficacy of adoptive T cell therapy by co-administering a blocking anti-CD47 monoclonal antibody (αCD47), based upon the hypothesis that macrophage mediated tumor phagocytosis is orthogonal to T cell mediated tumor killing and thus would provide at least an additive benefit. However, CD47 blockade completely abrogated the activity of adoptive T cell therapy through macrophage mediated depletion of the transferred T cells, in a manner sufficiently rapid and complete to serve as a safety switch in a lethal, autoreactive CAR T cell model.

Thus, to overcome this challenge, the present disclosure delivers a mechanism for selective CD47 blockade on tumor cells, but not T cells, to prevent macrophage mediated depletion of T cells and thereby enable benefit from simultaneous T cell and macrophage mediated antitumor effects. Demonstrated herein is the creation and expression on T cells of an engineered CD47 (47_{E}) that retains SIRPα signaling but is not blocked by αCD47. Adoptive transfer of T cells expressing 47_{E} administered with αCD47 induced sustained high levels of macrophages in the TME and dramatically enhanced antitumor activity. These results demonstrate that the antagonistic effects of T cell plus macrophage targeting therapies can be converted into synergistic effects when approaches are incorporated to prevent macrophage mediated phagocytosis of T cells.

### Materials and Methods

### Cell Lines

The Nalm6 B-ALL cell line was provided by David Barrett (Children's Hospital of Philadelphia) and retrovirally transduced to express GFP and firefly luciferase. 143B osteosarcoma cells (ATCC) were retrovirally transduced with human CD19. CHLA-255 neuroblastoma line was provided by Robert Seeger (Children's Hospital Los Angeles) and retrovirally transduced with GFP and firefly luciferase. MG63.3 was provided by Chand Khanna (National Cancer Institute, National Institutes of Health) and retrovirally transduced with GFP and firefly luciferase. D425 was provided by S. Chesier (Stanford University, Stanford, CA) and retrovirally transduced to express GFP and firefly luciferase. A375 melanoma cells were obtained from ATCC. The 293GP retroviral packaging line was provided by the Surgery Branch (National Cancer Institute, National Institutes of Health). Expi293 protein production cells were obtained from ATCC. D425 cells were maintained in serum-free media supplemented with B27 (Thermo Fisher Scientific), EGF, FGF (Shenandoah Biotechnology), human recombinant LIF (Millipore), and Heparin (StemCell Technologies). Nalm6, 143B, A375, MG63.3, and CHLA-255 were cultured in RPMI-1640 (Gibco). 293GP were cultured in DMEM (Gibco). Expi293 cells were cultured in Expi293 media (Thermo Fisher Scientific). Cell line culture media was supplemented with 10% FBS, 10 mM HEPES, 2 mM L-glutamine, 100 U/mL penicillin, and 100 μg/mL streptomycin (Gibco), except for Expi293 media. STR DNA profiling of all cell lines was conducted once per year (Genetica Cell Line testing). All cell lines were routinely tested for mycoplasma. Cell lines were cultured at 37° C. in a 5% CO₂ environment.

### Source of Primary Human T Cells and Macrophages

Buffy coats from healthy donors were purchased from the Stanford Blood Center under an IRB-exempt-protocol. Leukopaks from healthy donors were purchased from STEMCELL Technologies. Primary human T cells were purified by negative selection using the RosetteSep Human T cell Enrichment kit (Stem Cell Technologies) and SepMate-50 tubes. T cells were cryopreserved at 2×10⁷ cells per mL in CryoStor CS10 cryopreservation media (Stem Cell Technologies) until use. Primary peripheral monocytes were purified through successive density gradients using Ficoll (Sigma-Aldrich) and Percoll (GE Healthcare). Monocytes were then differentiated into macrophages by 7-9 d of culture in IMDM+10% AB human serum (Life Technologies).

### Viral Vector Construction

All retroviral constructs were cloned into the MSGV1 retroviral vector⁷⁴. B7H3.BBζ was generated by fusing, from N to C terminus, a human GM-CSF leader sequence, scFv derived from MGA271 in the VH-VL orientation and (GGGS)₃ linker sequence, CD8α hinge and transmembrane sequence, and human 4-1 BB and CD3ζ intracellular signaling domains. GD2.BBζ, Her2.BBζ, and CD19.BBζ were generated by cloning scFvs derived from 14G2A, 4D5, and FMC63 antibodies, respectively into the B7H3.BBζ vector. CD19.28ζ was generated by replacing the 4-1BB domain in CD19.BBζ with the intracellular signaling domain of human CD28. PIP.28ζ and PIP.BBζ were generated by replacing the FMC63 scFv with the 2.5F knottin⁷⁵ followed by a FLAG tag sequence (DYKDDDDK) in the CD19.28ζ and CD19.BBζ vectors, respectively. The in vivo T cell activation reporter was constructed by cloning a sequence containing firefly luciferase into the pGreenFirel-NF-κB lentiviral vector (System Biosciences) under the NF-κB responsive promoter. CD47 vectors were generated by inserting codon-optimized CD47 sequences (mutant and wild-type) in place of the CD19.BBζ sequence. For in vivo tracking, CAR-nLuc plasmids were generated by replacing the stop codon in the CD3ζ with a sequence containing a porcine teschovirus-1 2A (P2A) ribosomal skipping sequence, followed by nanoluciferase. Antares plasmids were generated by inserting the Antares sequence⁷³ in place of the CD19.BBζ sequence. The NY-ESO-1 TCR construct was generated by inserting the NY-ESO-1 α chain, followed by a P2A sequence, followed by the R chain in place of CD19.BBζ.

### Virus Production

Retroviral supernatant was packaged using 293GP cells and the RD114 envelope plasmid. In brief, 11 μg RD114 and 22 μg of the corresponding MSGV1 transfer plasmid were delivered to 293GP cells grown on 150 mm poly-D-lysine dishes (Corning) to 80% confluency by transient transfection with Lipofectamine 2000 (Thermo Fisher). Media was replenished every 24 hours. Virus production was performed side-by-side for comparable CAR, TCR, and CD47 constructs. Retroviral supernatant was harvested 48 and 72-hour post transfection. Supernatant from replicate dishes were pooled, centrifuged to deplete cell debris, and stored at −80° C. until use. Third-generation, self-inactivating lentiviral supernatant was similarly produced with 293T cells using 7 μg pMD2.G (VSVg) envelope, 18 μg pMDLg/pRRE (Gag/Pol), 18 μg pRSV-Rev, and 20 μg the corresponding transfer plasmids.

### CAR T and TCR T Manufacturing

At Day 0, primary human T cells were thawed and activated with anti-CD3/CD28 Human T-Expander Dynabeads (Thermo Fisher) at a 3:1 or 1:1 bead to cell ratio. On Day 2 virus coated culture plates were prepared on non-TC-treated 12-well plates that had been pre-coated with RetroNectin (Takara Bio) according to the manufacturer's instructions, by incubating with 1 mL of retroviral supernatant (2×10⁷-5×10⁷ TU/mL) and centrifugation at 3200 RPM, 32° C. for two hours. The supernatant was subsequently aspirated off of the wells and 0.5×10⁶ T cells were added in 1 mL of T cell media comprised of: AIM V (Thermo Fisher), 5% fetal bovine serum (FBS), 100 U/mL penicillin (Gibco), 100 mg/mL streptomycin (Gibco), 2 mM L-glutamine (Gibco), 10 mM HEPES (Gibco), and 40 U/mL rhIL-2 (Peprotech). After addition of the T cells, the plates were gently spun down at 1200 RPM for 2 min then incubated for 24 hrs at 37° C. 5% CO₂. This transduction process was repeated at Day 3 and Day 4 (if necessary). Dynabeads were removed on Day 4 or Day 5 by magnetic separation. Cells were maintained between 0.4-2×10⁶ cells/mL and expanded until Day 10-12. Typically, T cells were transduced with CAR or TCR on Day 2, and then CD47 variants on Days 3 and 4.

### Flow Cytometry

Recombinant B7H3-Fc and Her2-Fc (R&D systems) were used to detection B7H3 and Her2 surface CAR, respectively. Likewise, anti-FMC63 and anti-14g2a idiotype antibodies were used to detect CD19 and GD2 CAR, respectively. CAR detection reagents were fluorescently labeled with the DyLight 650 Microscale Antibody Labeling Kit (Thermo Fisher). Anti-FLAG (BioLegend) was used to detect the PIP CAR. NY-ESO-1 TCR was detected with antibodies specific for Vp13.1 (BioLegend), the beta chain of the NY-ESO-1 TCR. CD47 was detected with B6H12 (BD and Invitrogen), TJC4, Hu5F9, CV-1-Fc, mSIRPα-Fc (Sino Biological), or hSIRPα-Fc (Sino Biological), followed by detection with polyclonal anti-mouse- or human-IgG antibodies. The following antibodies were used for detection of cell-surface proteins: calreticulin (clone FMC 75; Abcam); human CD4 (clone SK3; BD); human CD8 (clone SK1; BD); human CD45 (clone H130; Thermo Fisher); human CD69 (clone FN50; BioLegend); human CD39 (clone A1; BioLegend); human TIM3 (clone F38-2E2; BioLegend); human LAG3 (clone 3DS223H; Invitrogen); human PD1 (clone J105; Invitrogen); human CD45RA (clone H1100; BioLegend); human CD62L (clone DREG-56; BD); human CD3 (clone SK7; BD); mouse CD45 (clone 13/2.3; BD); F4/80 (clone BM8; BioLegend); CD11b (clone M1/70; BD). Annexin V was detected using an eBioscience Annexin V Apoptosis Detection Kit (Invitrogen). Surface protein was stained by incubation with 3 μg/mL of detection reagents (or at the concentrations indicated in the figures) for 30 min at 4° C. Flow cytometry was performed on BD Fortessa and BD Accuri instruments.

### Bioluminescence Imaging

Mice were administered either 200 μL of 15 mg/mL D-luciferin or a 1:40 dilution of Nano-Glo substrate (Promega, diluted in DPBS) by intraperitoneal injection for firefly luciferase and Antares or nanoluciferase imaging, respectively. Images were acquired on an IVIS or Lago imaging system 4 min after injection for fLuc and 8 min after injection for nLuc/Antares using 30 sec exposures and medium binning. If saturated pixels were detected in the image, an additional image was acquired using the auto-expose setting. Total flux was measured using Living Image (Perkin Elmer) or Aura (Spectral Instruments Imaging) software with a region of interest around the body of each mouse. Only non-saturated images were used for quantification of BLI. Mice were randomized prior to T cell administration to ensure uniform distribution of tumor burden between groups. At the end of the experiment, all images were collected into a single sequence on Aura and set to the same luminescence scale.

### Recombinant Protein Cloning and Production

The gWIZ vector with a BM40 signal peptide was used for protein expression. DNA encoding Hu5F9's (magrolimab's) heavy chain with an hIgG1 Fc domain, Hu5F9's light chain, TJC4's (lemzoparlimab's) heavy chain with an hIgG1 Fc domain, and TJC4's light chain were ordered from Integrated DNA Technologies. Heavy and light chains were individually cloned into AscI/BamHI digested gWIZ vector using Gibson assembly. Plasmids were transfected into Expi293F cells (Thermo Fisher Scientific) in a 1:1 ratio of heavy chain:light chain using ExpiFectamine according to the manufacturer's instructions. Five days after transfection, supernatant was harvested, adjusted to pH 8.0 and sterile-filtered. Hu5F9 and TJC4 were then purified using recombinant Protein A-Sepharose 4B (Thermo Fisher Scientific) buffer exchanged into PBS and concentrated using Amicon Centrifugal Filters (Millipore Sigma). To assess CD47 binding, cells were stained with Hu5F9 or TJC4 and then stained with labeled anti-human secondary antibody (Invitrogen). B6H12 and mIgG1 isotype control (clone MOPC-21) were acquired from Bio X Cell. CV-1 variants (ALX-222 and ALX-90) was acquired from ALX Oncology. Human SIRPα-mFc and mouse SIRPα-hFc were acquired from Sino Biologic.

### Animal Models

NSG mice (NOD.Cg-Prkdc^{scid} Il12rgt^{m1Wjl}/SzJ) were purchased from the Jackson Laboratory and bred in house under Stanford University APLAC-approved protocols. Healthy male and female mice were used for in vivo experiments between 6 and 10 weeks old at tumor engraftment and were drug naïve, and not involved in previous procedures. Mice were housed in sterile cages in a barrier facility at Stanford University with a 12-hour light/dark cycle. Veterinary Services Center (VSC) staff at Stanford University monitored the mice daily and were euthanized when mice manifested persistent hunched posture, persistent scruffy coat, paralysis, impaired mobility, greater than 20% weight loss, if tumors significantly interfered with normal bodily functions, or if they exceeded limits designated in APLAC-approved protocols. Per recommendation by VSC staff, mice with morbidities were supported with 500 μL subcutaneous saline, diet gel (DietGel® 76A, ClearH2O), and wet chow.

### 143B Osteosarcoma Tumor Model

0.5×10⁶ or 1×10⁶ 143B or 143B-CD19 cells (143B cells engineered to over-express CD19; 143B cells do not naturally express CD19) in 100 μL DPBS were injected into the tibial periosteum of six- to ten-week-old NSG male or female mice (engraftment dose indicated in figure legends). Generally, five days after tumor implantation and after visual confirmation of tumor formation, mice were treated with Her2.BBζ-CAR T cells, followed by two doses of B6H12. Tumor progression was monitored by caliper measurement. Mice were euthanized according to the criteria described in the Animal Models section. Specifics for different iterations of the model presented are as follows:

CAR T+B6H12 studies (FIG. 1): mice engrafted with 0.5×10⁶ 143B-CD19 cells were treated with 10×10⁶ Her2.BBζ CAR T cells by tail vein injection on day 5. Mice were then treated twice with B6H12 (250 μg) or PBS by intraperitoneal injection on day 6 and day 10. T cells were quantified in the blood by flow cytometry on day 12.

47_{E}-CAR T studies with high-dose B6H12 (FIG. 42-44): mice engrafted with 1×10⁶ 143B-CD19 cells were treated with 4×10⁶ Her2.BBζ-Antares CAR T cells with endogenous CD47 knocked-out (47_{KO}) and over-expressing either CD47_{WT} (47_{WT}) or CD47 Q31P (47_{E}), or an equivalent number of mock-Antares T cells intravenously by tail vein injection on day 5. Mice were then treated twice with B6H12 (250 μg) or PBS by intraperitoneal injection on day 7 and day 11. T cells were quantified by nanoluciferase BLI before (day 7) and after (day 13) αCD47 treatment and in the blood by flow cytometry on day 14.

47_{E}-CAR T studies with low-dose B6H12 (FIG. 45-46): mice engrafted with 0.5×10⁶ 143B-CD19 cells were treated with 4×10⁶ Her2.BBζ-CAR T cells with endogenous CD47 knocked-out (47_{KO}) and over-expressing either CD47_{WT} (47_{WT}) or CD47 Q31P (47_{E}), or an equivalent number of mock T cells intravenously by tail vein injection on day 5. Mice were then treated twice with B6H12 (75 μg or 25 μg) or PBS by intraperitoneal injection on day 6 and day 10. T cells were quantified in the blood by flow cytometry on day 12. Only those mice treated with 47_{E}-CAR T cells were evaluated for antitumor efficacy in combination with B6H12.

### A375 Melanoma Tumor Model

3×10⁶ A375 cells in 100 μL DPBS were injected into the flanks of six- to ten-week-old NSG male or female mice. Generally, seven to fourteen days after tumor implantation and after visual confirmation of tumor formation, mice were treated with NY-ESO-1-TCR T cells, followed by two or three doses of B6H12. Tumor progression was monitored by caliper measurement. Mice were euthanized according to the criteria described in the Animal Models section. Specifics for different iterations of the model presented are as follows:

Low-dose NY-ESO-1 TCR T+B6H12 studies (FIG. 6): mice were treated with 2×10⁶ NY-ESO-1-TCR T cells, or an equivalent number of mock T cells intravenously by tail vein injection on day 9. Mice were then treated twice with B6H12 (250 μg) or PBS by intraperitoneal injection on day 10 and 15. T cells were quantified in the blood by flow cytometry on day 17.

High-dose NY-ESO-1 TCR T+B6H12 studies (FIG. 7): mice were treated with 5×10⁶ NY-ESO-1-TCR T cells, or an equivalent number of mock T cells intravenously by tail vein injection on day 7. Mice were then treated twice with B6H12 (250 μg) or PBS by intraperitoneal injection on day 9 and 13. T cells were quantified in the blood by flow cytometry on day 16.

47_{E}-NY-ESO-1-TCR T cell quantification studies (FIG. 50): Seven days after tumor implantation, mice were treated with 2.75×10⁶ NY-ESO-1-Antares TCR T cells with endogenous CD47 knocked-out (47_{KO}) and over-expressing CD47 Q31P (47_{E}), or an equivalent number of mock-Antares T cells intravenously by tail vein injection. Mice were then treated three times with B6H12 (250 μg) or PBS by intraperitoneal injection on day 9, 11, and 14. T cells were quantified by nanoluciferase BLI before (day 9) and after (day 14) αCD47 treatment and in the blood by flow cytometry on day 15.

47_{E}-NY-ESO-1-TCR T antitumor efficacy studies (FIG. 51): Seven days (T cell donor experiment 1; FIG. 51C) or fourteen days (T cell donor experiment 2; FIG. 51B) after tumor implantation, mice were treated with 1×10⁶ NY-ESO-1-Antares TCR T cells with endogenous CD47 knocked-out (47_{KO}) and over-expressing CD47 Q31P (47_{E}), or an equivalent number of mock-Antares T cells intravenously by tail vein injection. Mice were then treated either: (experiment 1) three times with B6H12 (250 μg) or PBS by intraperitoneal injection on day 9, 11, and 14, or (experiment 2) twice with B6H12 (250 μg) or PBS by intraperitoneal injection on day 15 and 19.

### MG63.3 Osteosarcoma Tumor Model

1×10⁶ MG63.3 cells in 100 μL DPBS were injected into the tibia periostea of six- to ten-week-old NSG male or female mice. Starting fifteen days after tumor implantation and after visual confirmation of tumor formation, mice were treated with 400 μg of B6H12 or PBS three times per week by intraperitoneal injection. On day 21, mice were treated with 10×10⁶ GD2.BBζ or B7H3.BBζ CAR T cells or no T cells. Tumor progression was measured with digital calipers twice per week. Mice were euthanized according to the criteria described in the Animal Models section.

### D425 Medulloblastoma Tumor Model

Six- to ten-week-old mice were anesthetized with 3% isoflurane (Minrad International) in an induction chamber. Anesthesia on the stereotactic frame (David Kopf Instruments) was maintained at 2% isoflurane delivered through a nose adaptor. D425 medulloblastoma cells were injected at coordinates 2 mm posterior to lambda on midline and 2 mm deep using a blunt-ended needle (75N, 26s/2″/2, 5 μL; Hamilton Co.). Using a microinjection pump (UMP-3; World Precision Instruments), 0.2×10⁶ D425-GL cells were injected in a volume of 3 μL at 30 nL/s. After leaving the needle in place for 1 minute, it was retracted at 3 mm/min. Four days after tumor implantation and after confirmation of tumor formation by bioluminescence, mice were randomized and treated with no T cells (B6H12 only group), or 10×10⁶ B7H3.BBζ CAR⁺ T cells or an equivalent number of non-tumor targeting CD19.BBζ CAR⁺ T cells intravenously by tail vein injection. Starting on day 4, mice were also treated with 400 μg of B6H12 or PBS three times per week by intraperitoneal injection. Tumor progression was monitored by firefly luciferase BLI.

### Nalm6 Leukemia Tumor Models

Six- to ten-week-old NSG male or female mice were implanted with 1×10⁶ Nalm6-GL cells by tail vein injection. CAR specificity, treatment doses and times for the specific model, and antibody doses are indicated in the figure legends. Mice treated with B6H12 were dosed with 250 μg/dose IP. Mice treated with CV-1 (ALX-90) were dosed with 400 μg/dose IP. Tumor progression was monitored by firefly luciferase BLI. T cells were quantified by nanoluciferase BLI before and after αCD47 treatment and in the blood by flow cytometry, as indicated. Mice were euthanized according to the criteria described in the Animal Models section.

### T Cell Depletion Model

Six- to ten-week-old NSG male or female mice were implanted with 2×10⁶ or 5×10⁶ CD19.28ζ-nLuc CAR T cells by tail vein injection (day 0). Mice were then treated twice with B6H12 (250 μg) or PBS by intraperitoneal injection on day 3 and day 5. T cells were quantified by nanoluciferase BLI before (2×10⁶ dose: day 2; 5×10⁶ dose: day 3) and after (2×10⁶ dose: day 9; 5×10⁶ dose: day 7) anti-CD47 treatment and in the blood by flow cytometry (2×10⁶ dose: day 7; 5×10⁶ dose: day 6). For isotype control studies (FIG. 5A-B) mice were implanted with 5×10⁶ CD19.28ζ-CAR T cells by tail vein injection (day 0), and then treated with B6H12 (250 μg), mIgG1 isotype control (250 μg) or PBS by intraperitoneal injection on day 1. T cells were quantified in the blood by flow cytometry on day 5. Mice were euthanized according to the criteria described in the Animal Models section at the conclusion of the experiment.

### CHLA-255 Neuroblastoma Metastatic Tumor Model

Six- to ten-week-old NSG male or female mice were implanted with 1×10⁶ CHLA-255-GL cells by tail vein injection. Seven days after tumor implantation and after confirmation of tumor formation by bioluminescence, mice were randomized and treated with 2×10⁶ B7H3.BBζ-nLuc CAR T cells with endogenous CD47 knocked out (47_{KO}) and over-expressing either CD47_{WT} (47_{WT}) or CD47 Q31P (47_{E}) or an equivalent number of mock (non-transduced) T cells intravenously by tail vein injection. Mice were then treated three times with B6H12 (250 μg) or PBS by intraperitoneal injection on day 7, day 9, and day 13. Tumor progression was monitored by firefly luciferase BLI. T cells were quantified by nanoluciferase BLI after αCD47 treatment on day 14 and in the blood by flow cytometry on day 15. Mice were euthanized according to the criteria described in the Animal Models section.

Isolation of T Cells from Spleens and Tumors

Spleens and tumors were harvested and mechanically dissociated using a gentleMACS dissociator (Miltenyi). Single-cell suspensions were made by passing spleens and tumors through a 70 μm cell strainer, depleting red blood cells by ACK lysis (Quality Biological Inc.), and further filtration through flow cytometry filter tubes with 35 μm cell strainer caps (Falcon). Single cell suspensions were then frozen in CryoStor buffer in liquid nitrogen, or stained and run directly on flow cytometry, staining for Live/Dead, hCD3, hCD45, hCD4, hCD8, as well as CAR.

Quantification of T Cells and Cytokines from Blood

Mouse blood was collected from the retro-orbital sinus into Microvette blood collection tubes with EDTA (Fisher Scientific). Red blood cells were depleted by ACK lysis (Quality Biological Inc.), followed by two washes with FACS buffer (PBS+2% FBS). Samples were stained with anti-hCD45, anti-hCD4, anti-hCD8, anti-hCD47, and anti-CAR reagents. Samples were mixed with CountBright Absolute Counting beads (Thermo Fisher) before flow cytometry analysis

### CRISPR/Cas9 Knock-Out of CD47

Ribonucleoprotein (RNP) was prepared using synthetic sgRNA with 2′-O-methyl phosphorothioate modification (Synthego) diluted in TE buffer at 120 μM. Five microliters sgRNA were incubated with 2.5 μl duplex buffer (IDT) and 2.5 μg Alt-R S.p. Cas9 Nuclease V3 (IDT) for 30 min at room temperature. One hundred-microliter reactions were assembled with 5 million T cells, 90 μl P3 buffer (Lonza), and 10 μl RNP. Cells were pulsed with protocol E0115 using the P3 Primary Cell 4D-Nucleofector Kit and 4D-Nucleofector System (Lonza). Cells were recovered immediately with warm media for 6 hours before transduction with CAR. Guide sequence: CD47-sg3: 5′ AUGCUUUGUUACUAAUAUGG 3′ (SEQ ID NO:41)

### Incucyte Tumor Killing Assays, Cytokine Analysis, and T Cell Activation Marker Detection

5×10⁴ GFP-labeled tumor cells were cocultured with 5×10⁴ CAR T cells in 200 μL RPMI supplemented with 10% FBS, 10 mM HEPES, 2 mM L-glutamine, 100 U/mL penicillin, and 100 μg/mL streptomycin. For conditions with B6H12, a concentration of 10 μg/mL was used. Triplicate wells were plated in 96-well flat-bottom plates for each condition. Tumor fluorescence was monitored every 2-3 hours with a 10× objective using the Incucyte Zoom system (Essen Bioscience), housed in a cell culture incubator at 37° C. and 5% CO₂, set to take 4 images per well at each time point. Total integrated GFP intensity was quantified using the Zoom software (Essen Bioscience). Data were normalized to the first timepoint and plotted as fold change in tumor fluorescence over time. For cytokine secretion and T cell marker analysis, cocultures were set up as above except in 96-well round bottom plates. After approximately 24 hours, plates were spun down to pellet cells and 150 μL of supernatant was harvested and stored at −80° C. until analysis, while cell pellets were immediately processed for flow cytometry. IFNγ and IL-2 levels in coculture supernatants were quantified by ELISA (Human ELISA MAX Deluxe, Biolegend) according to the manufacturer's instructions. Negative cytokine values were set to 0. For analysis of T cell markers after activation by tumor cells, pellets from spun plates were pooled together for triplicate wells, stained for live cells, hCD4, hCD8, Annexin V, hCD69, hCD47, hPD1, hTIM3, hLAG3, and hCD39, and analyzed by flow cytometry. Coculture experiments were setup using day 10 T cells.

### Macrophage Depletion and Peritoneal Lavage

Six- to ten-week-old NSG male or female mice were pre-treated by intravenous injection with 200 μL of clodronate liposomes (Liposoma), followed by 400 μg of anti-CSF1R (Bio X Cell, AFS98) by intraperitoneal injection. Mice were treated with 400 μg of anti-CSF1R three times per week for the duration of the experiment. Six days after clodronate treatment, mice were administered with 2×10⁶ CD19.28ζ-nLuc CAR T cells, followed by 250 μg B6H12 on day 7. T cells were quantified by nanoluciferase BLI before (day 7) and after (day 9) anti-CD47 treatment. Peritoneal lavage was performed on day 13 with 10 mL of FACS buffer and a 25-gauge needle. Peritoneal cells were collected and stained for Live/Dead, CD11 b, F4/80, hCD45, and mCD45, before being run on flow cytometry.

### Phagocytosis Assay

For all flow-based in vitro phagocytosis assays, T cells and human macrophages were co-cultured at a ratio of 2:1 (e.g. 100,000 T cells: 50,000 macrophages) in ultra-low-attachment 96-well U-bottom plates (Corning) in serum-free RPMI (Thermo Fisher Scientific). T cells were labeled with CFSE (Invitrogen) by suspending cells in PBS (5 μM working solution) as per manufacturer instructions for 20 min at 37° C. protected from light and washed twice with 20 ml of FBS-containing media before co-culture. Cells were then either incubated alone or in the presence of anti-CD47 (clone B6H12; Bio X Cell) or mIgG1 isotype control (clone MOPC-21; Bio X Cell) at a concentration of 10 μg ml-1. T cells and antibodies were incubated for 30 min in a humidified 5% CO₂ incubator at 37° C. Plates were washed two times; human macrophages were added to the plate; and plates were incubated for 1-2 h at 37° C. Phagocytosis was stopped by washing with 4° C. PBS and centrifugation at 1450 rpm before the cells were stained with Live/Dead stain and anti-CD11 b-APC. Assays were analyzed by flow cytometry, and phagocytosis was measured as the number of CD11b⁺ and CFSE⁺ macrophages, quantified as a percentage of the total CD11b⁺ macrophages and normalized to the control condition.

For Incucyte-based in vitro phagocytosis assays, T cells and human macrophages were co-cultured at a ratio of 2:1 (e.g. 100,000 T cells: 50,000 macrophages) in 96-well flat-bottom plates (Corning) in RPMI supplemented with 10% FBS, 10 mM HEPES, 2 mM L-glutamine, 100 U/mL penicillin, and 100 μg/mL streptomycin. T cells were labeled with pHrodo Red dye (Invitrogen) by incubating T cells at 1×10⁶ cells/mL with a working concentration of pHrodo Red of 30 ng/mL in PBS for 1 h at 37° C. in the dark in a humidified 5% CO₂ incubator. The labeling reaction was quenched and excess dye washed away by washing twice with complete media. Cells were then either incubated alone or in the presence of anti-CD47 (clone B6H12; Bio X Cell) at a concentration of 10 μg ml⁻¹ in serum-free RPMI. T cells and antibodies were incubated for 30 min in a humidified 5% CO₂ incubator at 37° C., before being washed two times. Macrophages were added to each plate well and allowed to adhere for 2 h in a humidified 5% CO₂ incubator at 37° C. After 2 h, labeled T cells were added to the plate at a 2:1 T cell:macrophage ratio. pHrodo Red fluorescence due to phagocytosis was monitored after 3 hours with a 10× objective using the Incucyte Zoom system (Essen Bioscience), housed in a cell culture incubator at 37° C. and 5% CO₂, set to take 4 images per well. Total integrated RFP intensity was quantified using the Zoom software (Essen Bioscience).

### Quantification of CD47 Expression on Tumor and T Cells Using QuantiBrite

CD47 expression was quantified using an anti-CD47-PE antibody (clone B6H12; BD) and a QuantiBrite PE Quantitation Kit (BD) following the manufacturer's instructions⁷⁶. CD19.28ζ-CAR T cells were produced as described above, save that cells were kept in culture one day after thawing prior to activation with anti-CD3/CD28 beads. T cells were analyzed by flow cytometry on day 0 (prior to activation; one day after thaw), day 4 (immediately after removal from bead activation), day 7, and day 11 (average time of transfer in vivo). T cells were stained with anti-hCD4, anti-hCD8, anti-hCD47 or mIgG1 isotype control (cloneB11/6; Abcam), anti-hCD45RA, and anti-hCD62L. T cell differentiation subtypes were defined as: T naïve (CD45RA⁺/CD62L⁺; T_{N}), T central memory (CD45RA⁻/CD62L⁺; T_{CM}), T effector memory (CD45RA⁻/CD62L; T_{EM}), and T effector memory re-expressing CD45RA (CD45RA⁺/CD62L; T_{EMRA}). Tumor cells were stained with only anti-hCD47 or mIgG1 isotype control. Molecules of CD47 were calculated as per QuantiBrite kit instructions using extrapolation from MFI signals of BD QuantiBrite-PE beads with known quantities of PE. The degree of labeling for anti-CD47-PE (BD Lot #: 2040745) was determined experimentally as 0.842 molecules of dye per antibody, using the maximum absorbance at 566 nm, the extinction coefficient for PE (1,863,000 M⁻¹ cm⁻¹), and the listed antibody concentration.

### Imaging of Patient CSF Samples

A cerebrospinal fluid cytospin preparation was collected from a patient treated with axicabtagene ciloleucel (axi-cel) CD19.28ζ CAR T cell therapy, stained with Wright Giemsa, and imaged via microscopy at 1000× magnification, capturing histiocytes with engulfed lymphocytes.

### Single Cell Analysis of Patient Samples

Two datasets were re-analyzed: Good, Z., et. al. 2022⁵¹: scRNA-seq data collected from nine LBCL patients treated with axicabtagene ciloleucel (axi-cel) CD19.28ζ CAR T cell therapy, where 50,000-70,000 CAR T cells (single live CD4⁺/or CD8α⁺/CD235a⁻/CAR⁺ events) were FACS sorted to ≥95% purity and analyzed on the 10× Genomics platform^{51,77} (GSE168940); and Majzner, R. G., et. al. 2022⁵²: scRNA-seq data collected from four DMG patients treated with GD2.BBζ CAR T cell therapy, where cells from the manufacturing product and CSF were analyzed on the 10× Genomics platform^{52,77} (GSE186802). Where indicated, previously annotated CAR mRNA-expressing cells were used.

### Histology of Tissue Samples

The tissues assessed include skin and lung. Tissues were harvested and immersion-fixed in 10% neutral buffered formalin. After fixation, tissues were routinely processed, embedded in paraffin, sectioned at 5.0 μm and routinely stained with hematoxylin and eosin (H&E). Tissues were visualized with an Olympus BX43 upright bright-field microscope, and images were captured using an Olympus DP27 camera and cellSens software.

### Yeast Surface Display Vectors

A DNA sequence encoding the CD47 Ig-like domain (Gln19-Ser135) was cloned into the pCTCON2 yeast-surface display vector (Addgene) using the NheI and BamHI sites. The pFreeNTerm (pFNT) vector was based on the pCL backbone⁷⁸, designing an intrinsic NheI cutsite into the Aga2p signal sequence as the 5′ cloning site and using a MluI cutsite prior to a Gly₄Ser 3× linker as the 3′ cloning site. The CD47 Ig-like domain (Gln19-Ser135) was cloned into the pFNT yeast-surface display vector using these NheI and MluI sites.

### Yeast Surface Display Binding Assays

EBY100 yeast were transformed with pCTCON2 or pFNT plasmids and selected on SD-CAA-Agar plates. Yeast (~100,000 per sample) were grown and induced in SG-CAA, and binding set up over a range of soluble ligand or receptor concentrations in phosphate-buffered saline (PBS) containing 1 mg ml⁻¹ bovine serum albumin (BSA; BPBS), taking into account ligand depletion and equilibrium time⁷⁹. After incubation with binding partner, yeast cells were washed once with BPBS, then incubated with a 1:5,000 dilution of chicken anti-c-myc antibody (A21281, Invitrogen) for pCTCON2 displayed proteins, and incubated for 30 min at 4° C. in the dark. After primary addition, samples were washed once with BPBS, and secondary antibodies were added. Expression was detected with a 1:500 dilution of goat anti-chicken Alexa Fluor 488 or Alexa Fluor 647 (Invitrogen). For pFNT displayed proteins, co-displayed GFP was used to monitor expression. Binding of proteins with mouse Fc domains (hSIRPα, B6H12) was detected with a 1:500 dilution of goat anti-mouse Alexa Fluor 488 or Alexa Fluor 647 (Invitrogen). Binding of proteins with a human Fc domain (CV-1 [ALX-222], mSIRPα, Hu5F9, TJC4) was detected using a 1:500 dilution of goat anti-human Alexa Fluor 488 or Alexa Fluor 647 (Invitrogen). Secondary antibodies were incubated for 15 min at 4° C. in the dark. After secondary incubation, samples were washed once with BPBS, pelleted, and left pelleted on ice until analysis. Samples were analyzed by resuspending them in 50 μL of BPBS and running flow cytometry using a BD Accuri C6 (BD Biosciences). Samples were gated for bulk yeast cells (forward scatter (FSC) vs. side scatter (SSC)) and then for single cells (FSC-Height vs. FSC-Area). Expressing yeast were determined and gated via C-terminal c-myc tag or GFP detection. The geometric mean of the binding fluorescence signal was quantified from the expressing population and used as a raw binding value. When comparing binding signals, the average fluorescence expression signal was quantified for different protein variants and used to normalize binding signal. To determine “fraction bound,” binding signals were divided by the signal derived from the highest concentration of binding partner used, or that derived from binding to wild-type CD47. To calculate K_{d} values, data were analyzed in GraphPad Prism (v9.3.1) using non-linear regression curve fit.

### Yeast Surface Display Library Generation, Sorting, and Sequencing

CD47 was expressed in Saccharomyces cerevisiae (strain: EBY100; ATCC MYA-4941) as a genetic fusion to the agglutinin mating protein Aga2p. An error-prone PCR library was created using the CD47 Ig-like domain (Gln19 to Ser135) as a template and mutations were introduced with a Gene Morph II random mutagenesis kit (Aglilent), following the manufacturer's instructions. Separate PCRs were performed using various concentrations of Mutazyme II enzyme. Products from these reactions were purified via gel electrophoresis, pooled, and amplified with standard PCR using Phusion polymerase (New England BioLabs). Purified mutant DNA and linearized plasmid were electroporated into EBY100 yeast, where they were assembled in vivo through homologous recombination. We estimated 5×10⁷ variants for the library, determined by dilution plating and colony counting. Yeast were grown in SD-CAA media and induced for CD47 protein expression by growth in media containing 90% SG-CAA and 10% SD-CAA overnight⁷⁹. Yeast displaying CD47 variants were isolated via fluorescence-activated cell sorting (FACS) using a SONY SH800S cell sorter (SONY) and analyzed with a BD Accuri C6 flow cytometer (BD Biosciences). Data were analyzed using FlowJo software (v 10.6.1, Tree Star Inc.). Screens were carried out using equilibrium binding conditions where yeast were incubated at room temperature in BPBS with the following concentrations of B6H12 or CV-1 (ALX-222) for two hours. For negative sorts to B6H12, the CD47-expressing, but non-binding populations of yeast were collected. For positive sorts to CV-1, the CD47-expressing and binding populations of yeast were collected. Sort 1, negative sort, 500 μM B6H12; Sort 2, negative sort, 5 nM B6H12; Sort 3, positive sort, 20 nM CV-1; Sort 4, negative sort, 20 nM B6H12; Sort 5, negative sort, 50 nM B6H12; Sort 6, positive sort, 10 nM CV-1. After incubation with B6H12 or CV-1, yeast were pelleted, washed, and labeled with fluorescent antibodies as described above prior to sorting. Sorted yeast clones were propagated, induced for CD47 expression, and subjected to iterative rounds of FACS as described above. After each round of screening, plasmid DNA was recovered using a Zymoprep yeast plasmid miniprep I kit (Zymo Research Corp), transformed into DH10B electrocompetent cells (Thermo Fisher), and isolated using a GeneJET plasmid miniprep kit (Thermo Fisher). Sequencing was performed by ELIM Biopharmaceuticals, Inc. (Hayward, CA).

### CD47 Structure Modeling

CD47 structures were downloaded from the protein data bank (PDB) and analyzed using PyMol. The CD47-hSIRPα structure used was 2JJS⁵⁴. The CD47-B6H12 structure used was 5TZU⁵⁵.

### 143B Correlative Study and Tumor Dissociation

1×10⁶ 143B cells in 100 μL DPBS were injected into the tibia periosteum of six- to ten-week-old NSG mice. Thirteen days after tumor implantation and after visual confirmation of tumor formation, mice were treated with 4×10⁶ Her2.BBζ-CAR T cells with endogenous CD47 knocked-out (47_{KO}) and over-expressing either CD47_{WT} (47_{WT}) or CD47 Q31P (47_{E}), an equivalent number of Mock T cells intravenously by tail vein injection, or no T cells. Mice were then treated twice with B6H12 (250 μg) or PBS by intraperitoneal injection on day 15 and day 19. Tumor progression was monitored by caliper measurement. Tumors were harvested at day 21 post tumor implantation (day 8 post CAR T treatment). Tumors were weighed and then split with a razor, with one section being fixed in 10% paraformaldehyde, and the other mechanically dissociated as described above, before being stained for flow cytometry and FACS. Formaldehyde fixed tumor had paraformaldehyde removed after 24 h and replaced with 70% ethanol for long term storage. Tumor sections were then formalin-fixed and paraffin-embedded following the standard protocol.

### Flow Cytometry and IHC on Dissociated Tumors

Flow cytometry: Tumors were harvested as above. Single cell suspensions of dissociated tumors were stained for CAR (Her2-Fc; R&D), hCD19 (BD), CD11b (BD), F4/80 (BioLegend), hCD45 (Invitrogen), hCD3 (BD), mCD45 (BD), hCD47 (BD), and Live/Dead (Invitrogen) for 30 minutes in PBS+2% FBS (FACS Buffer) before being analyzed by flow cytometry.

IHC: Tumors were harvested as above. Formalin-fixed, paraffin-embedded xenograft tumor sections were used. F4/80 (Cell Signaling Technology) staining was performed manually, and hCD3 (Abcam) and Arg1 (Cell Signaling Technology) staining was performed using the Ventana Discovery platform. In brief, tissue sections were incubated in either 6 mM citrate buffer (F4/80) or Tris EDTA buffer (CD3/Arg1, 1:100 and 1:250 dilution respectively) (cell conditioning 1 standard) at 100° C. for 25 min (F480) or 95° C. for 1 h (CD3/Arg1) to retrieve antigenicity, followed by incubation with the respective primary antibody for 1 h. Bound primary antibodies were incubated with the respective secondary antibodies (Vector Laboratories or Jackson Laboratory) with 1:500 dilution, followed by UltraMap HRP and Vectore Lab (F4/80) or ChromoMap DAB (CD3/Arg1) detection. For IHC analysis, tumor regions were identified based on histology. F4/80, CD3, and Arg1 positivity were analyzed for each tumor region. F4/80, CD3, and Arg1 IHC positivity scores were automatically quantified in the regions of interest with Aperio ImageScope software. Regions of interest were randomly selected within the tumor to exclude macrophages present in the normal tissue around the tumor.

### Single Cell Analysis of Dissociated 143B Tumors

Dissociated tumors from the 143B osteosarcoma model described above were sorted for live cells using a Live/Dead stain (Invitrogen) at the Stanford Shared FACS facility. Single-cell RNAseq libraries were prepared using the Chromium Next GEM Single Cell 5′ v2 platform (10× GENOMICS). Libraries were sent to Novogene for sequencing on a NovoSeq S4 lane (PE150) with approximately 30,000 mean reads per cell. Reads were aligned and quantified with Cell Ranger (10× GENOMICS) using the standard workflow, with the reference transcriptomes GRCh38 for human and mm10 for mouse. The Cell Ranger output was imported into R using Seurat 4.2.0. The following filters were applied using the subset function to select for live cells: nFeature_RNA >200 & nFeature_RNA <5000; percent mitochondrial reads <5%. After filtering, the eight biological samples ranged from 7658-9327 mean unique molecular identifiers (UMI) per cell. The data matrix was normalized with NormalizeData and scaled with Seurat. Differential expression analysis, clustering, and UMAP dimensionality reduction analysis were performed on the resulting data matrix using Seurat⁸⁰. Pathway analysis was performed using Enrichr⁸¹.

### Statistical Analyses

The specific statistical tests utilized are indicated in the figure legends. Statistical analyses were performed using Prism (v 9.3.1, GraphPad Software). For comparisons between two groups, statistical significance was assayed by two-tailed unpaired Student's t-test or a Mann-Whitney test. For comparison within in vivo studies and between grouped studies, a two-way analysis of variance (ANOVA) combined with Tukey's multiple comparison test for post hoc analysis was performed. Significance for survival data was calculated using the log-rank Mantel-Cox test.

Sample sizes were determined on the basis of the variability of tumor models used. Tumor-bearing animals were assigned to the treatment groups to ensure an equal distribution of tumor sizes between groups. Data are represented as mean±standard deviation (in vitro studies) or mean±standard error of the mean (some in vivo studies). For all statistical analyses, P values are indicated in each figure panel.

### REFERENCES

1 DeNardo, D. G. & Ruffell, B. Macrophages as regulators of tumour immunity and immunotherapy. Nat Rev Immunol 19, 369-382 (2019). doi.org:10.1038/s41577-019-0127-6
2 Barry, S. T., Gabrilovich, D. I., Sansom, O. J., Campbell, A. D. & Morton, J. P. Therapeutic targeting of tumour myeloid cells. Nat Rev Cancer 23, 216-237 (2023). doi.org:10.1038/s41568-022-00546-2
3 Kloosterman, D. J. & Akkari, L. Macrophages at the interface of the co-evolving cancer ecosystem. Cell 186 (2023). doi.org:10.1016/j.cell.2023.02.020
4 Shen, H. et al. Prognostic Value of Tumor-Associated Macrophages in Clear Cell Renal Cell Carcinoma: A Systematic Review and Meta-Analysis. Front Oncol 11, 657318 (2021). doi.org:10.3389/fonc.2021.657318
5 Li, J. et al. Tumor-associated macrophage infiltration and prognosis in colorectal cancer: systematic review and meta-analysis. Int J Colorectal Dis 35, 1203-1210 (2020). doi.org:10.1007/s00384-020-03593-z
6 Ries, C. H. et al. Targeting tumor-associated macrophages with anti-CSF-1R antibody reveals a strategy for cancer therapy. Cancer Cell 25, 846-859 (2014). doi.org:10.1016/j.ccr.2014.05.016
7 Zhu, Y. et al. CSF1/CSF1R blockade reprograms tumor-infiltrating macrophages and improves response to T-cell checkpoint immunotherapy in pancreatic cancer models. Cancer Res 74, 5057-5069 (2014). doi.org:10.1158/0008-5472.CAN-13-3723
8 Xu, J. et al. CSF1R signaling blockade stanches tumor-infiltrating myeloid cells and improves the efficacy of radiotherapy in prostate cancer. Cancer Res 73, 2782-2794 (2013). doi.org:10.1158/0008-5472.CAN-12-3981
9 Noel, M. et al. Phase 1b study of a small molecule antagonist of human chemokine (C-C motif) receptor 2 (PF-04136309) in combination with nab-paclitaxel/gemcitabine in first-line treatment of metastatic pancreatic ductal adenocarcinoma. Invest New Drugs 38, 800-811 (2020). doi.org:10.1007/s10637-019-00830-3
10 Chen, Y. L. Prognostic significance of tumor-associated macrophages in patients with nasopharyngeal carcinoma: A meta-analysis. Medicine (Baltimore) 99, e21999 (2020). doi.org:10.1097/MD.0000000000021999
11 Matlung, H. L., Szilagyi, K., Barclay, N. A. & van den Berg, T. K. The CD47-SIRPalpha signaling axis as an innate immune checkpoint in cancer. Immunol Rev 276, 145-164 (2017). doi.org:10.1111/imr.12527
12 Majeti, R. et al. CD47 is an adverse prognostic factor and therapeutic antibody target on human acute myeloid leukemia stem cells. Cell 138, 286-299 (2009). doi.org:10.1016/j.cell.2009.05.045
13 Chao, M. P. et al. Anti-CD47 antibody synergizes with rituximab to promote phagocytosis and eradicate non-Hodgkin lymphoma. Cell 142, 699-713 (2010). doi.org:10.1016/j.cell.2010.07.044
14 Willingham, S. B. et al. The CD47-signal regulatory protein alpha (SIRPα) interaction is a therapeutic target for human solid tumors. Proceedings of the National Academy of Sciences of the United States of America 109, 6662-6667 (2012). doi.org:10.1073/PNAS.1121623109/-/DCSUPPLEMENTAL
15 Advani, R. et al. CD47 Blockade by Hu5F9-G4 and Rituximab in Non-Hodgkin's Lymphoma. New England Journal of Medicine 379, 1711-1721 (2018). doi.org:10.1056/NEJMOA1807315/SUPPL_FILE/NEJMOA1807315_DISCLOSURES.P DF
16 Sallman, D. A. et al. Magrolimab in Combination With Azacitidine in Patients With Higher-Risk Myelodysplastic Syndromes: Final Results of a Phase Ib Study. J Clin Oncol, JC02201794 (2023). doi.org:10.1200/JCO.22.01794
17 Sikic, B. 1. et al. First-in-Human, First-in-Class Phase I Trial of the Anti-CD47 Antibody Hu5F9-G4 in Patients With Advanced Cancers. Journal of Clinical Oncology 37, 946-946 (2019). doi.org:10.1200/JCO.18.02018
18 Lakhani, N. J. et al. Evorpacept alone and in combination with pembrolizumab or trastuzumab in patients with advanced solid tumours (ASPEN-01): a first-in-human, open-label, multicentre, phase 1 dose-escalation and dose-expansion study. Lancet Oncol 22, 1740-1751 (2021). doi.org:10.1016/S1470-2045(21)00584-2
19 Neelapu, S. S. et al. Axicabtagene Ciloleucel CAR T-Cell Therapy in Refractory Large B-Cell Lymphoma. N Engl J Med 377, 2531-2544 (2017). doi.org:10.1056/NEJMoa1707447
20 Fry, T. J. et al. CD22-targeted CAR T cells induce remission in B-ALL that is naive or resistant to CD19-targeted CAR immunotherapy. Nat Med 24, 20-28 (2018). doi.org:10.1038/nm.4441
21 Maude, S. L. et al. Tisagenlecleucel in Children and Young Adults with B-Cell Lymphoblastic Leukemia. N Engl J Med 378, 439-448 (2018). doi.org:10.1056/NEJMoa1709866
22 Abramson, J. S. et al. Lisocabtagene maraleucel for patients with relapsed or refractory large B-cell lymphomas (TRANSCEND NHL 001): a multicentre seamless design study. Lancet 396, 839-852 (2020). doi.org:10.1016/S0140-6736(20)31366-0
23 Wang, M. et al. KTE-X19 CAR T-Cell Therapy in Relapsed or Refractory Mantle-Cell Lymphoma. N Engl J Med 382, 1331-1342 (2020). doi.org:10.1056/NEJMoa1914347
24 Berdeja, J. G. et al. Ciltacabtagene autoleucel, a B-cell maturation antigen-directed chimeric antigen receptor T-cell therapy in patients with relapsed or refractory multiple myeloma (CARTITUDE-1): a phase Ib/2 open-label study. Lancet 398, 314-324 (2021). doi.org:10.1016/S0140-6736(21)00933-8
25 Munshi, N. C. et al. Idecabtagene Vicleucel in Relapsed and Refractory Multiple Myeloma. N Engl J Med 384, 705-716 (2021). doi.org:10.1056/NEJMoa2024850
26 Fowler, N. H. et al. Tisagenlecleucel in adult relapsed or refractory follicular lymphoma: the phase 2 ELARA trial. Nat Med 28, 325-332 (2022). doi.org:10.1038/s41591-021-01622-0
27 Kamdar, M. et al. Lisocabtagene maraleucel versus standard of care with salvage chemotherapy followed by autologous stem cell transplantation as second-line treatment in patients with relapsed or refractory large B-cell lymphoma (TRANSFORM): results from an interim analysis of an open-label, randomised, phase 3 trial. Lancet 399, 2294-2308 (2022). doi.org:10.1016/S0140-6736(22)00662-6
28 Locke, F. L. et al. Axicabtagene Ciloleucel as Second-Line Therapy for Large B-Cell Lymphoma. N Engl J Med 386, 640-654 (2022). doi.org:10.1056/NEJMoa2116133
29 Nastoupil, L. J. et al. Standard-of-Care Axicabtagene Ciloleucel for Relapsed or Refractory Large B-Cell Lymphoma: Results From the US Lymphoma CAR T Consortium. J Clin Oncol 38, 3119-3128 (2020). doi.org:10.1200/JCO.19.02104
30 Schultz, L. M. et al. Disease Burden Affects Outcomes in Pediatric and Young Adult B-Cell Lymphoblastic Leukemia After Commercial Tisagenlecleucel: A Pediatric Real-World Chimeric Antigen Receptor Consortium Report. J Clin Oncol 40, 945-955 (2022). doi.org:10.1200/JCO.20.03585
31 Labanieh, L. & Mackall, C. L. CAR immune cells: design principles, resistance and the next generation. Nature 2023 614:7949 614, 635-648 (2023). doi.org:10.1038/s41586-023-05707-3
32 Siegel, R. L., Miller, K. D. & Jemal, A. Cancer statistics, 2020. CA Cancer J Clin 70, 7-30 (2020). doi.org:10.3322/caac.21590
33 Schmidts, A. & Maus, M. V. Making CAR T Cells a Solid Option for Solid Tumors. Front Immunol 9, 2593 (2018). doi.org:10.3389/fimmu.2018.02593
34 Labanieh, L. et al. Enhanced safety and efficacy of protease-regulated CAR-T cell receptors. Cell 185, 1745-1763.e1722 (2022). doi.org:10.1016/J.CELL.2022.03.041
35 Freitas, K. A. et al. Enhanced T cell effector activity by targeting the Mediator kinase module. Science 378 (2022). doi.org:10.1126/SCIENCE.ABN5647/SUPPL_FILE/SCIENCE.ABN5647_MDAR_REPR ODUCIBILITY_CHECKLIST.PDF
36 Gresham, H. D., Goodwin, J. L., Allen, P. M., Anderson, D. C. & Brown, E. J. A novel member of the integrin receptor family mediates Arg-Gly-Asp-stimulated neutrophil phagocytosis. Journal of Cell Biology 108, 1935-1943 (1989). doi.org:10.1083/JCB.108.5.1935
37 Lo, J. et al. Anti-CD47 antibody suppresses tumour growth and augments the effect of chemotherapy treatment in hepatocellular carcinoma. Liver International 36, 737-745 (2016). doi.org:10.1111/LIV.12963/SUPPINFO
38 Kaur, S. et al. A function-blocking CD47 antibody suppresses stem cell and EGF signaling in triple-negative breast cancer. Oncotarget 7, 10133-10152 (2016). doi.org:10.18632/ONCOTARGET.7100
39 Theruvath, J. et al. Anti-GD2 synergizes with CD47 blockade to mediate tumor eradication. Nature Medicine 2022 28:2 28, 333-344 (2022). doi.org:10.1038/s41591-021-01625-x
40 Majzner, R. G. et al. CAR T cells targeting B7-H3, a pan-cancer antigen, demonstrate potent preclinical activity against pediatric solid tumors and brain tumors. Clinical Cancer Research 25, 2560-2574 (2019). doi.org:10.1158/1078-0432.CCR-18-0432/73008/AM/CAR-T-CELLS-TARGETING-B7-H3-A-PAN-CANCER-ANTIGEN
41 Thomas, R. et al. NY-ESO-1 Based Immunotherapy of Cancer: Current Perspectives. Frontiers in Immunology 9, 947-947 (2018). doi.org:10.3389/FIMMU.2018.00947
42 Musolino, A. et al. Role of Fcγ receptors in HER2-targeted breast cancer therapy. (2022). doi.org:10.1136/jitc-2021-003171
43 Osorio, J. C., Smith, P., Knorr, D. A. & Ravetch, J. V. The antitumor activities of anti-CD47 antibodies require Fc-FcγR interactions. Cancer Cell 0 (2023). doi.org:10.1016/j.ccell.2023.10.007
44 Lo, M. et al. Effector-attenuating Substitutions That Maintain Antibody Stability and Reduce Toxicity in Mice. The Journal of Biological Chemistry 292, 3900-3900 (2017). doi.org:10.1074/JBC.M116.767749
45 Weiskopf, K. et al. Engineered SIRPα variants as immunotherapeutic adjuvants to anticancer antibodies. Science 341, 88-91 (2013). doi.org:10.1126/SCIENCE.1238856/SUPPL_FILE/WEISKOPF.SM.PDF
46 Hu, X. et al. Engineered Hypoimmune Allogeneic CAR T Cells Exhibit Innate and Adaptive Immune Evasion Even after Sensitization in Humanized Mice and Retain Potent Anti-Tumor Activity. Blood 138, 1690-1690 (2021). doi.org:10.1182/BLOOD-2021-150021
47 Jaiswal, S. et al. CD47 Is Upregulated on Circulating Hematopoietic Stem Cells and Leukemia Cells to Avoid Phagocytosis. Cell 138, 271-285 (2009). doi.org:10.1016/J.CELL.2009.05.046/ATTACHMENT/C76B88E8-DOAF-494A-9683-993A7A88A224/MMC1. PDF
48 Chao, M. P. et al. Calreticulin is the dominant pro-phagocytic signal on multiple human cancers and is counterbalanced by CD47. Science translational medicine 2, 63ra94-63ra94 (2010). doi.org:10.1126/SCITRANSLMED.3001375
49 N N, S. & A M, H. Hemophagocytosis in cerebrospinal fluid after CAR T-cell therapy. Blood 139 (2022). doi.org:10.1182/blood.2021014630
50 S, S., D, L., CJ, V. & CY, H. Cytomorphologic Features Found in Cerebrospinal Fluid Specimens of Hemophagocytic Lymphohistiocytosis Patients. American journal of clinical pathology 156 (2021). doi.org:10.1093/ajcp/aqaa248
51 Good, Z. et al. Post-infusion CAR TReg cells identify patients resistant to CD19-CAR therapy. Nature Medicine 2022 28:9 28, 1860-1871 (2022). doi.org:10.1038/s41591-022-01960-7
52 Majzner, R. G. et al. GD2-CAR T cell therapy for H3K27M-mutated diffuse midline gliomas. Nature 603, 934-941 (2022). doi.org:doi:10.1038/s41586-022-04489-4
53 Ho, C. C. M. et al. “Velcro” Engineering of High Affinity CD47 Ectodomain as Signal Regulatory Protein α (SIRPα) Antagonists That Enhance Antibody-dependent Cellular Phagocytosis *. Journal of Biological Chemistry 290, 12650-12663 (2015). doi.org:10.1074/JBC.M115.648220
54 Hatherley, D. et al. Paired receptor specificity explained by structures of signal regulatory proteins alone and complexed with CD47. Molecular cell 31, 266-277 (2008). doi.org:10.1016/J.MOLCEL.2008.05.026
55 Pietsch, E. C. et al. Anti-leukemic activity and tolerability of anti-human CD47 monoclonal antibodies. Blood Cancer Journal 7, e536-e536 (2017). doi.org:10.1038/BCJ.2017.7
56 Fenalti, G. et al. Structure of the human marker of self 5-transmembrane receptor CD47. Nature communications 12 (2021). doi.org:10.1038/S41467-021-25475-W
57 Mehta, A. et al. Lemzoparlimab, a Differentiated Anti-CD47 Antibody in Combination with Rituximab in Relapsed and Refractory Non-Hodgkin's Lymphoma: Initial Clinical Results. Blood 138, 3542-3542 (2021). doi.org:10.1182/BLOOD-2021-150606
58 Li, M. et al. Anti-CD47 immunotherapy in combination with BCL-2 inhibitor to enhance anti-tumor activity in B-cell lymphoma. Hematological Oncology 40, 596-608 (2022). doi.org:10.1002/HON.3009
59 Weiskopf, K. et al. CD47-blocking immunotherapies stimulate macrophage-mediated destruction of small-cell lung cancer. The Journal of Clinical Investigation 126, 2610-2610 (2016). doi.org:10.1172/JC181603
60 Meng, Z., Wang, Z., Guo, B., Cao, W. & Shen, H. TJC4, a Differentiated Anti-CD47 Antibody with Novel Epitope and RBC Sparing Properties. Blood134, 4063-4063 (2019). doi.org:10.1182/BLOOD-2019-122793
61 Appay, V. & Rowland-Jones, S. L. RANTES: a versatile and controversial chemokine. Trends in Immunology 22, 83-87 (2001). doi.org:10.1016/S1471-4906(00)01812-3
62 Menten, P., Wuyts, A. & Van Damme, J. Macrophage inflammatory protein-1. Cytokine & Growth Factor Reviews 13, 455-481 (2002). doi.org:10.1016/S1359-6101(02)00045-X
63 Kersten, K. et al. Spatiotemporal co-dependency between macrophages and exhausted CD8+ T cells in cancer. Cancer Cell 40, 624-638.e629 (2022). doi.org:10.1016/J.CCELL.2022.05.004
64 Fitzgerald, K. A., O'Neill, L. A. J. & Gearing, A. J. H. The Cytokine Factsbook and Webfacts (2nd Edition). 526-526 (2001).
65 Zou, J. J. et al. Structure-Function Analysis of the p35 Subunit of Mouse Interleukin 12. Journal of Biological Chemistry 270, 5864-5871 (1995). doi.org:10.1074/jbc.270.11.5864
66 Grewal, I. S. & Flavell, R. A. The role of CD40 ligand in costimulation and T-cell activation. Immunological reviews 153 (1996). doi.org:10.1111/j.1600-065x.1996.tb00921.x
67 Kuhn, N. F. et al. CD40 Ligand-Modified Chimeric Antigen Receptor T Cells Enhance Antitumor Function by Eliciting an Endogenous Antitumor Response. Cancer Cell 35, 473-488.e476 (2019). doi.org:10.1016/j.ccell.2019.02.006
68 Duan, Z. & Luo, Y. Targeting macrophages in cancer immunotherapy. Signal Transduction and Targeted Therapy 2021 6:1 6, 1-21 (2021). doi.org:10.1038/s41392-021-00506-6
69 Zhang, Z. et al. Role of lysosomes in physiological activities, diseases, and therapy. Journal of Hematology & Oncology 14, 1-39 (2021). doi.org:doi:10.1186/s13045-021-01087-1
70 Yao, Y., Xu, X. H. & Jin, L. Macrophage polarization in physiological and pathological pregnancy. Frontiers in Immunology 10, 792-792 (2019). doi.org:10.3389/FIMMU.2019.00792/BIBTEX
71 Zizzo, G., Hilliard, B. A., Monestier, M. & Cohen, P. L. Efficient Clearance of Early Apoptotic Cells by Human Macrophages Requires M2c Polarization and MerTK Induction. The Journal of Immunology 189, 3508-3520 (2012). doi.org:10.4049/JIMMUNOL.1200662
72 Roszer, T. Understanding the mysterious M2 macrophage through activation markers and effector mechanisms. Mediators of Inflammation 2015 (2015). doi.org:10.1155/2015/816460
73 Chu, J. et al. A bright cyan-excitable orange fluorescent protein facilitates dual-emission microscopy and enhances bioluminescence imaging in vivo. Nature Biotechnology 2016 34:734, 760-767 (2016). doi.org:10.1038/nbt.3550
74 Hughes, M. S. et al. Transfer of a TCR Gene Derived from a Patient with a Marked Antitumor Response Conveys Highly Active T-Cell Effector Functions. home.liebertpub.com/hum 16, 457-472 (2005). doi.org:10.1089/HUM.2005.16.457
75 Miller, C. L. et al. Systemic delivery of a targeted synthetic immunostimulant transforms the immune landscape for effective tumor regression. Cell Chemical Biology 29, 451-462.e458 (2022). doi.org:10.1016/J.CHEMBIOL.2021.10.012
76 Radosevich, M. T. et al. Antigen density quantification of cell-surface immunotherapy targets by flow cytometry: Multi-antigen assay of neuroblastoma bone marrow metastasis. STAR protocols 4 (2023). doi.org:10.1016/j.xpro.2023.102709
77 Hao, Y. et al. Integrated analysis of multimodal single-cell data. Cell 184, 3573-3587.e3529 (2021). doi.org:10.1016/J.CELL.2021.04.048/ATTACHMENT/1E5EB5C1-59EE-4B2B-8BFA-14B48A54FF8F/MMC3.XLSX
78 Lim, S., Glasgow, J. E., Filsinger Interrante, M., Storm, E. M. & Cochran, J. R. Dual display of proteins on the yeast cell surface simplifies quantification of binding interactions and enzymatic bioconjugation reactions. Biotechnology Journal 12, 1600696-1600696 (2017). doi.org:10.1002/BIOT.201600696
79 Hunter, S. A. & Cochran, J. R. Vol. 580 21-44 (Academic Press Inc., 2016).
80 Hafemeister, C. & Satija, R. Normalization and variance stabilization of single-cell RNA-seq data using regularized negative binomial regression. Genome Biology 20, 1-15 (2019). doi.org:10.1186/S13059-019-1874-1/FIGURES/6
81 Chen, E. Y. et al. Enrichr: interactive and collaborative HTML5 gene list enrichment analysis tool. BMC bioinformatics 14 (2013). doi.org:10.1186/1471-2105-14-128

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A nucleic acid encoding a CD47 polypeptide, wherein the CD47 polypeptide comprises a mutant CD47 Ig-like domain that reduces binding of a therapeutic anti-CD47 binding agent to the CD47 polypeptide as compared to binding of the therapeutic anti-CD47 binding agent to a wild-type CD47 polypeptide, and wherein the CD47 polypeptide retains binding to SIRPα.

2. The nucleic acid of claim 1, wherein the mutant CD47 Ig-like domain comprises a mutant BC loop.

3. The nucleic acid of claim 2, wherein the mutant BC loop comprises an amino acid substitution at E29, A30, Q31, or any combination thereof, wherein numbering is according to the amino acid sequence set forth in SEQ ID NO:1.

4. The nucleic acid of claim 3, wherein the mutant BC loop comprises the amino acid substitution E29A, A30P, Q31P/Q31A, or any combination thereof.

5. The nucleic acid of any one of claims 1 to 4, wherein the therapeutic anti-CD47 binding agent is a therapeutic anti-CD47 antibody.

6. The nucleic acid of claim 5, wherein the therapeutic anti-CD47 antibody comprises, or competes for binding to CD47 with an antibody comprising, the six complementarity determining regions (CDRs) of lemzoparlimab or antibody B6H12.

7. The nucleic acid of claim 5, wherein the therapeutic anti-CD47 antibody comprises, or competes for binding to CD47 with an antibody comprising, the six complementarity determining regions (CDRs) of magrolimab.

8. The nucleic acid of any one of claims 1 to 7, wherein the mutant CD47 Ig-like domain enhances binding of the CD47 polypeptide to SIRPα as compared to binding of a wild-type CD47 polypeptide to SIRPα.

9. A CD47 polypeptide encoded by the nucleic acid of any one of claims 1 to 8.

10. An expression construct comprising the nucleic acid of any one of claims 1 to 8.

11. A cell comprising the expression construct of claim 10, wherein the cell expresses the CD47 polypeptide on its surface.

12. A cell comprising the nucleic acid of any one of claims 1 to 8, wherein the nucleic acid is a transgene integrated into the genome of the cell or maintained episomally in the cell, and wherein the cell expresses the CD47 polypeptide on its surface.

13. The cell of claim 12, wherein the transgene comprises the nucleic acid operably linked to one or more expression control sequences.

14. The cell of claim 12, wherein the transgene is operably linked to an endogenous promoter of the cell.

15. A cell comprising the nucleic acid of any one of claims 1 to 8, wherein the nucleic acid is the endogenous CD47 gene of the cell which has been mutated to encode the CD47 polypeptide comprising the mutant CD47 Ig-like domain, and wherein the cell expresses the CD47 polypeptide on its surface.

16. The cell of any one of claims 11 to 15, wherein the cell is a therapeutic immune cell.

17. The cell of claim 16, wherein the therapeutic immune cell is a therapeutic T cell, a therapeutic natural killer T (NKT) cell, a therapeutic natural killer (NK), or a therapeutic macrophage.

18. The cell of claim 16, wherein the therapeutic immune cell is a therapeutic T cell.

19. The cell of any one of claims 16 to 18, wherein the therapeutic immune cell comprises a nucleic acid that encodes an engineered receptor, wherein the therapeutic immune cell further expresses the engineered receptor on its surface.

20. The cell of claim 19, wherein the engineered receptor is a chimeric antigen receptor (CAR), a T cell receptor (TCR), a synthetic Notch (SynNotch) receptor, a Modular Extracellular Sensor Architecture (MESA) receptor, a Tango receptor, a ChaCha receptor, a generalized extracellular molecule sensor (GEMS) receptor, a cytokine receptor, a chemokine receptor, a switch receptor, an adhesion molecule, an integrin, an inhibitory receptor, a stimulatory receptor, an immunoreceptor tyrosine-based activation motif (ITAM)-containing receptor, or an immunoreceptor tyrosine-based inhibition motif (ITIM)-containing receptor.

21. The cell of claim 19, wherein the engineered receptor is a CAR.

22. The cell of any one of claims 19 to 21, wherein the engineered receptor comprises an extracellular binding domain that binds to a tumor antigen.

23. The cell of any one of claims 19 to 21, wherein the engineered receptor comprises an extracellular binding domain that binds to CD47.

24. The cell of any one of claims 16 to 18, wherein the therapeutic immune cell is a tumor infiltrating lymphocyte (TIL).

25. The cell of any one of claims 16 to 24, wherein the therapeutic immune cell does not express, or exhibits reduced expression of, endogenous wild-type CD47.

26. The cell of claim 25, wherein the therapeutic immune cell comprises a knockout of the endogenous wild-type CD47 gene.

27. The cell of claim 25, wherein the therapeutic immune cell exhibits reduced expression of endogenous wild-type CD47 via endogenous wild-type CD47 knockdown.

28. The cell of any one of claims 16 to 27, wherein the therapeutic immune cell comprises an expression construct that encodes the therapeutic anti-CD47 binding agent, wherein the therapeutic T cell expresses and secretes the therapeutic anti-CD47 binding agent.

29. The cell of claim 28, wherein the therapeutic anti-CD47 binding agent is a therapeutic anti-CD47 antibody.

30. A composition comprising a population of therapeutic immune cell as defined in any one of claims 16 to 29.

31. A method of administering an adoptive cell therapy to a subject having cancer and receiving an anti-CD47 therapy to treat the cancer, the method comprising administering to the subject the composition of claim 30 in an amount effective to treat the cancer.

32. The method according to claim 31, wherein the adoptive cell therapy is an adoptive T cell therapy.

33. The method according to claim 31 or claim 32, wherein the anti-CD47 therapy is a therapeutic anti-CD47 antibody therapy.

34. The method according to any one of claims 31 to 33, wherein the cancer comprises a solid tumor.

35. The method according to claim 34, wherein the solid tumor is a carcinoma, lymphoma, blastoma, or sarcoma.

36. The method according to claim 34 or claim 35, wherein the method produces a synergistic effect between the adoptive cell therapy and the anti-CD47 therapy.

37. The method according to any one of claims 31 to 33, wherein the cancer comprises a hematological malignancy.

38. The method according to claim 37, wherein the hematological malignancy is a leukemia, a lymphoma, or multiple myeloma.

39. The method according to any one of claims 31 to 33, wherein the cancer is myelodysplastic syndrome (MDS), acute myeloid leukemia (AML), multiple myeloma (MM), Non-Hodgkin's lymphoma (NHL), non-small cell lung cancer, head and neck squamous cell carcinoma, gastroesophageal junction adenocarcinoma, gastric adenocarcinoma, diffuse large B cell lymphoma, follicular lymphoma, marginal zone lymphoma, mantle cell lymphoma, chronic lymphocytic lymphoma (CLL), B cell lymphoma, lung adenocarcinoma, osteosarcoma, ovarian cancer, or leiomyosarcoma.


SEQUENCE LISTING (41 sequences)

SEQ ID NO: 1 - protein, 305 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 2 - protein, 305 aa, synthetic construct
QLLFNKTKSVEFTFCNDTVVIPCFVTNMEPQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 3 - DNA, 969 bp, synthetic construct
  source 1..969: mol_type = other DNA
atgtggccgttggttgcagcactcttgcttggatctgcttgctgtggatccgctcaactcctttttaataagacgaaatccgtcgaatttacattttgcaatgataccgtcgttatcccatgctttgtcacgaacatggagccccagaatacgaccgaagtctacgtgaagtggaaatttaagggacgcgatatttatactttcgatggggcattgaacaaatccacagtcccaaccgacttttcctccgcgaaaatcgaagttagtcaacttcttaaaggagacgcgagtctcaagatggacaaatccgacgccgtgagtcatactggtaattacacctgcgaggtcacggaactgacacgagaaggggagactataatagaacttaaataccgagtcgtttcctggtttagtccgaatgagaatattctgattgttatatttccgatatttgccatcctgttgttctggggccaattcggaataaagactctcaagtatcgcagcgggggaatggacgaaaaaaccattgctctcctggtggccggtcttgtaattactgtgatagtgattgttggcgcaattctctttgtgccaggggaatattctctcaagaatgccactggattgggtttgatagttacctccacgggcatactgatccttcttcattactatgtatttagcacagccattggtcttacaagctttgttattgccatcctggtaattcaggtaatagcatacatcctcgcagtcgttggacttagcttgtgcatagcagcttgcatacctatgcacggtccactgttgatctcagggttgtccatactcgcactcgcgcagctgcttggtctcgtgtacatgaaattcgtagcatcaaaccaaaaaaccatacaaccgcccaggaaggctgtggaagaacccctgaacgcatttaaggaatccaaggggatgatgaacgatgag

SEQ ID NO: 4 - protein, 305 aa, synthetic construct
QLLFNKTKSVEFTFCNDTVVIPCFVTNMEAPNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 5 - DNA, 969 bp, synthetic construct
  source 1..969: mol_type = other DNA
atgtggccgttggttgcagcactcttgcttggatctgcttgctgtggatccgctcaactcctttttaataagacgaaatccgtcgaatttacattttgcaatgataccgtcgttatcccatgctttgtcacgaacatggaggccccgaatacgaccgaagtctacgtgaagtggaaatttaagggacgcgatatttatactttcgatggggcattgaacaaatccacagtcccaaccgacttttcctccgcgaaaatcgaagttagtcaacttcttaaaggagacgcgagtctcaagatggacaaatccgacgccgtgagtcatactggtaattacacctgcgaggtcacggaactgacacgagaaggggagactataatagaacttaaataccgagtcgtttcctggtttagtccgaatgagaatattctgattgttatatttccgatatttgccatcctgttgttctggggccaattcggaataaagactctcaagtatcgcagcgggggaatggacgaaaaaaccattgctctcctggtggccggtcttgtaattactgtgatagtgattgttggcgcaattctctttgtgccaggggaatattctctcaagaatgccactggattgggtttgatagttacctccacgggcatactgatccttcttcattactatgtatttagcacagccattggtcttacaagctttgttattgccatcctggtaattcaggtaatagcatacatcctcgcagtcgttggacttagcttgtgcatagcagcttgcatacctatgcacggtccactgttgatctcagggttgtccatactcgcactcgcgcagctgcttggtctcgtgtacatgaaattcgtagcatcaaaccaaaaaaccatacaaccgcccaggaaggctgtggaagaacccctgaacgcatttaaggaatccaaggggatgatgaacgatgag

SEQ ID NO: 6 - protein, 305 aa, synthetic construct
QLLFNKTKSVEFTFCNDTVVIPCFVTNMAAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 7 - DNA, 969 bp, synthetic construct
  source 1..969: mol_type = other DNA
atgtggccgttggttgcagcactcttgcttggatctgcttgctgtggatccgctcaactcctttttaataagacgaaatccgtcgaatttacattttgcaatgataccgtcgttatcccatgctttgtcacgaacatggcagcccagaatacgaccgaagtctacgtgaagtggaaatttaagggacgcgatatttatactttcgatggggcattgaacaaatccacagtcccaaccgacttttcctccgcgaaaatcgaagttagtcaacttcttaaaggagacgcgagtctcaagatggacaaatccgacgccgtgagtcatactggtaattacacctgcgaggtcacggaactgacacgagaaggggagactataatagaacttaaataccgagtcgtttcctggtttagtccgaatgagaatattctgattgttatatttccgatatttgccatcctgttgttctggggccaattcggaataaagactctcaagtatcgcagcgggggaatggacgaaaaaaccattgctctcctggtggccggtcttgtaattactgtgatagtgattgttggcgcaattctctttgtgccaggggaatattctctcaagaatgccactggattgggtttgatagttacctccacgggcatactgatccttcttcattactatgtatttagcacagccattggtcttacaagctttgttattgccatcctggtaattcaggtaatagcatacatcctcgcagtcgttggacttagcttgtgcatagcagcttgcatacctatgcacggtccactgttgatctcagggttgtccatactcgcactcgcgcagctgcttggtctcgtgtacatgaaattcgtagcatcaaaccaaaaaaccatacaaccgcccaggaaggctgtggaagaacccctgaacgcatttaaggaatccaaggggatgatgaacgatgag

SEQ ID NO: 8 - protein, 305 aa, synthetic construct
QLLFNKTKSVEFTFCNDTVVIPCFVTNMEPANTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 9 - DNA, 969 bp, synthetic construct
  source 1..969: mol_type = other DNA
atgtggccgttggttgcagcactcttgcttggatctgcttgctgtggatccgctcaactcctttttaataagacgaaatccgtcgaatttacattttgcaatgataccgtcgttatcccatgctttgtcacgaacatggagcccgcgaatacgaccgaagtctacgtgaagtggaaatttaagggacgcgatatttatactttcgatggggcattgaacaaatccacagtcccaaccgacttttcctccgcgaaaatcgaagttagtcaacttcttaaaggagacgcgagtctcaagatggacaaatccgacgccgtgagtcatactggtaattacacctgcgaggtcacggaactgacacgagaaggggagactataatagaacttaaataccgagtcgtttcctggtttagtccgaatgagaatattctgattgttatatttccgatatttgccatcctgttgttctggggccaattcggaataaagactctcaagtatcgcagcgggggaatggacgaaaaaaccattgctctcctggtggccggtcttgtaattactgtgatagtgattgttggcgcaattctctttgtgccaggggaatattctctcaagaatgccactggattgggtttgatagttacctccacgggcatactgatccttcttcattactatgtatttagcacagccattggtcttacaagctttgttattgccatcctggtaattcaggtaatagcatacatcctcgcagtcgttggacttagcttgtgcatagcagcttgcatacctatgcacggtccactgttgatctcagggttgtccatactcgcactcgcgcagctgcttggtctcgtgtacatgaaattcgtagcatcaaaccaaaaaaccatacaaccgcccaggaaggctgtggaagaacccctgaacgcatttaaggaatccaaggggatgatgaacgatgag

SEQ ID NO: 10 - protein, 305 aa, synthetic construct
QLLFNKTKSVEFTFCNDTVVIPCFVTNMAPANTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 11 - DNA, 969 bp, synthetic construct
  source 1..969: mol_type = other DNA
atgtggccgttggttgcagcactcttgcttggatctgcttgctgtggatccgctcaactcctttttaataagacgaaatccgtcgaatttacattttgcaatgataccgtcgttatcccatgctttgtcacgaacatggcacccgcgaatacgaccgaagtctacgtgaagtggaaatttaagggacgcgatatttatactttcgatggggcattgaacaaatccacagtcccaaccgacttttcctccgcgaaaatcgaagttagtcaacttcttaaaggagacgcgagtctcaagatggacaaatccgacgccgtgagtcatactggtaattacacctgcgaggtcacggaactgacacgagaaggggagactataatagaacttaaataccgagtcgtttcctggtttagtccgaatgagaatattctgattgttatatttccgatatttgccatcctgttgttctggggccaattcggaataaagactctcaagtatcgcagcgggggaatggacgaaaaaaccattgctctcctggtggccggtcttgtaattactgtgatagtgattgttggcgcaattctctttgtgccaggggaatattctctcaagaatgccactggattgggtttgatagttacctccacgggcatactgatccttcttcattactatgtatttagcacagccattggtcttacaagctttgttattgccatcctggtaattcaggtaatagcatacatcctcgcagtcgttggacttagcttgtgcatagcagcttgcatacctatgcacggtccactgttgatctcagggttgtccatactcgcactcgcgcagctgcttggtctcgtgtacatgaaattcgtagcatcaaaccaaaaaaccatacaaccgcccaggaaggctgtggaagaacccctgaacgcatttaaggaatccaaggggatgatgaacgatgag

SEQ ID NO: 12 - protein, 305 aa, synthetic construct
QLLFNKTKSVEFTFCNDTVVIPCFVTNMAPQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 13 - DNA, 969 bp, synthetic construct
  source 1..969: mol_type = other DNA
atgtggccgttggttgcagcactcttgcttggatctgcttgctgtggatccgctcaactcctttttaataagacgaaatccgtcgaatttacattttgcaatgataccgtcgttatcccatgctttgtcacgaacatggcaccccagaatacgaccgaagtctacgtgaagtggaaatttaagggacgcgatatttatactttcgatggggcattgaacaaatccacagtcccaaccgacttttcctccgcgaaaatcgaagttagtcaacttcttaaaggagacgcgagtctcaagatggacaaatccgacgccgtgagtcatactggtaattacacctgcgaggtcacggaactgacacgagaaggggagactataatagaacttaaataccgagtcgtttcctggtttagtccgaatgagaatattctgattgttatatttccgatatttgccatcctgttgttctggggccaattcggaataaagactctcaagtatcgcagcgggggaatggacgaaaaaaccattgctctcctggtggccggtcttgtaattactgtgatagtgattgttggcgcaattctctttgtgccaggggaatattctctcaagaatgccactggattgggtttgatagttacctccacgggcatactgatccttcttcattactatgtatttagcacagccattggtcttacaagctttgttattgccatcctggtaattcaggtaatagcatacatcctcgcagtcgttggacttagcttgtgcatagcagcttgcatacctatgcacggtccactgttgatctcagggttgtccatactcgcactcgcgcagctgcttggtctcgtgtacatgaaattcgtagcatcaaaccaaaaaaccatacaaccgcccaggaaggctgtggaagaacccctgaacgcatttaaggaatccaaggggatgatgaacgatgag

SEQ ID NO: 14 - protein, 305 aa, synthetic construct
QLLFNKTKSVEFTFCNDTVVIPCFVTNMAAPNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 15 - DNA, 969 bp, synthetic construct
  source 1..969: mol_type = other DNA
atgtggccgttggttgcagcactcttgcttggatctgcttgctgtggatccgctcaactcctttttaataagacgaaatccgtcgaatttacattttgcaatgataccgtcgttatcccatgctttgtcacgaacatggcagccccgaatacgaccgaagtctacgtgaagtggaaatttaagggacgcgatatttatactttcgatggggcattgaacaaatccacagtcccaaccgacttttcctccgcgaaaatcgaagttagtcaacttcttaaaggagacgcgagtctcaagatggacaaatccgacgccgtgagtcatactggtaattacacctgcgaggtcacggaactgacacgagaaggggagactataatagaacttaaataccgagtcgtttcctggtttagtccgaatgagaatattctgattgttatatttccgatatttgccatcctgttgttctggggccaattcggaataaagactctcaagtatcgcagcgggggaatggacgaaaaaaccattgctctcctggtggccggtcttgtaattactgtgatagtgattgttggcgcaattctctttgtgccaggggaatattctctcaagaatgccactggattgggtttgatagttacctccacgggcatactgatccttcttcattactatgtatttagcacagccattggtcttacaagctttgttattgccatcctggtaattcaggtaatagcatacatcctcgcagtcgttggacttagcttgtgcatagcagcttgcatacctatgcacggtccactgttgatctcagggttgtccatactcgcactcgcgcagctgcttggtctcgtgtacatgaaattcgtagcatcaaaccaaaaaaccatacaaccgcccaggaaggctgtggaagaacccctgaacgcatttaaggaatccaaggggatgatgaacgatgag

SEQ ID NO: 16 - protein, 18 aa, synthetic construct
MWPLVAALLLGSACCGSA

SEQ ID NO: 17 - protein, 5 aa, synthetic construct
GYGMS

SEQ ID NO: 18 - protein, 17 aa, synthetic construct
TITSGGTYTYYPDSVKG

SEQ ID NO: 19 - protein, 9 aa, synthetic construct
SLAGNAMDY

SEQ ID NO: 20 - protein, 11 aa, synthetic construct
RASQTISDYLH

SEQ ID NO: 21 - protein, 7 aa, synthetic construct
FASQSIS

SEQ ID NO: 22 - protein, 9 aa, synthetic construct
QNGHGFPRT

SEQ ID NO: 23 - protein, 5 aa, synthetic construct
RAWMN

SEQ ID NO: 24 - protein, 19 aa, synthetic construct
RIKRKTDGETTDYAAPVKG

SEQ ID NO: 25 - protein, 7 aa, synthetic construct
SNRAFDI

SEQ ID NO: 26 - protein, 17 aa, synthetic construct
KSSQSVLYAGNNRNYLA

SEQ ID NO: 27 - protein, 7 aa, synthetic construct
QASTRAS

SEQ ID NO: 28 - protein, 9 aa, synthetic construct
QQYYTPPLA

SEQ ID NO: 29 - protein, 5 aa, synthetic construct
NYNMH

SEQ ID NO: 30 - protein, 17 aa, synthetic construct
TIYPGNDDTSYNQKFKD

SEQ ID NO: 31 - protein, 8 aa, synthetic construct
GGYRAMDY

SEQ ID NO: 32 - protein, 16 aa, synthetic construct
RSSQSIVYSNGNTYLG

SEQ ID NO: 33 - protein, 7 aa, synthetic construct
KVSNRFS

SEQ ID NO: 34 - protein, 10 aa, synthetic construct
FQGSHVPYTF

SEQ ID NO: 35 - protein, 10 aa, synthetic construct
GYTFTNYWIH

SEQ ID NO: 36 - protein, 17 aa, synthetic construct
YTDPRTDYTEYNQKFKD

SEQ ID NO: 37 - protein, 8 aa, synthetic construct
GGRVGLGY

SEQ ID NO: 38 - protein, 16 aa, synthetic construct
RSSQNIVQSNGNTYLE

SEQ ID NO: 39 - protein, 7 aa, synthetic construct
KVFHRFS

SEQ ID NO: 40 - protein, 9 aa, synthetic construct
FQGSHVPYT

SEQ ID NO: 41 - RNA, 20 nt, synthetic construct
  source 1..20: mol_type = other RNA
atgctttgttactaatatgg